(19) United States
(12) Patent Application Publication
(10) Pub. No.: US 2025/0288654 A1
(43) Pub. Date: Sep. 18, 2025

(54) TOLERANCE-INDUCING CONSTRUCTS AND COMPOSITIONS AND THEIR USE FOR THE TREATMENT OF IMMUNE DISORDERS

(71) Applicant: Nykode Therapeutics ASA, Oslo (NO)

(72) Inventors: Agnete Brunsvik Fredriksen, Rælingen (NO); Heidi Myrset, Fetsund (NO); Audun Trygge Haugen Bersaas, Oslo (NO); Stine Granum, Oslo (NO); Pierre Dillard, Oslo (NO)

(73) Assignee: Nykode Therapeutics ASA, Oslo (NO)

(21) Appl. No.: 18/560,033

(22) PCT Filed: May 10, 2022
(86) PCT No.: PCT/EP2022/062629

§ 371 Date: Nov. 9, 2023

(30) Foreign Application Priority Data
May 10, 2021 (DK) PA 2021 70226
Jul. 8, 2021 (DK) PA 2021 70365

(51) Int. Cl. A61K 39/00; A61K 39/385; A61P 37/06; C07K 14/54; C07K 16/28

(52) U.S. Cl. CPC A61K 39/0008; A61K 39/385; A61P 37/06; C07K 14/5428; C07K 16/2851; A61K 2039/53; A61K 2039/577; A61K 2039/6056; C07K 2317/622

(57) ABSTRACT

The present disclosure relates to tolerance-inducing constructs for inducing tolerance, such as by targeting the tolerance-inducing construct to antigen presenting cells (APCs). Further disclosed are polynucleotides, vectors, host cells, pharmaceutical compositions and kits comprising said tolerance-inducing construct. Also disclosed are tolerance-inducing constructs and compositions for use in the treatment of immune disorders, such as in the prophylactic or therapeutic treatment of autoimmune diseases, allergic disease and graft rejection.

### CROSS-REFERENCE TO RELATED APPLICATIONS

This application is a National Stage Entry of PCT/EP2022/062629, filed May 10, 2022, which claims the benefit of priority to DK patent application No. 202170365, filed Jul. 8, 2021, and DK patent application No. 202170226, filed May 10, 2021, all of which are hereby incorporated by reference in their entirety.

### TECHNICAL FIELD

The present disclosure relates to constructs and compositions for use in the treatment of conditions involving undesired immune reactions, such as in the prophylactic or therapeutic treatment of autoimmune diseases, allergic disease and graft rejection.

### REFERENCE TO AN ELECTRONIC SEQUENCE LISTING

The instant application contains a Sequence Listing which has been submitted in ASCII (ST.25) format via USPTO Patent Center, which is hereby incorporated by reference into the specification in its entirety. Said ASCII copy, named “V89540_1130US_SL”, was created on May 29, 2024, and is 150,909 bytes in size.

### BACKGROUND

Immune responses are necessary for protection against diseases, e.g. diseases caused by pathogens like viruses, bacteria or parasites. However, undesirable immune activation can cause processes leading to damage or destruction of one's own tissues. Undesirable immune activation occurs, for example, in autoimmune diseases where antibodies and/or T lymphocytes react with self-antigens resulting in e.g. tissue damage and pathology. Undesirable immune activation also occurs in allergic reactions, which are characterized by an exaggerated immune response to typically harmless substances from the environment and which may result in inflammatory responses leading to tissue destruction. Further, undesired immune activation occurs in graft rejection, e.g., rejection of transplanted organs or tissue which is significantly mediated by alloreactive T cells present in the host, which T cells recognize donor alloantigens or xenoantigens; this leads to destruction of the transplanted organ or tissue.

Immune tolerance is the acquired lack of specific immune responses to substances or tissue that have the capacity to elicit an immune response in a given organism. Typically, to induce tolerance, to a specific antigen, the antigen must be presented by an antigen presenting cell (APC) to other immune cells in the absence of activation signals, which results in the death or functional inactivation of antigen specific effector lymphocytes or the generation of antigen-specific cells that maintain the tolerance. This process generally accounts for tolerance to self-antigens, or self-tolerance. Immunosuppressive drugs are useful in prevention or reduction of undesirable immune responses, e.g., in treating patients with autoimmune diseases or with allogeneic transplants. Conventional strategies for generating immunosuppression of an unwanted immune response are based on broad-acting immunosuppressive drugs. Additionally, to maintain immunosuppression, immunosuppressive drug therapy is often a life-long proposition. Unfortunately, the use of broad-acting immunosuppressive drugs is associated with a risk of severe side effects, such as immunodeficiency, because the majority of them act non-selectively, resulting in increased susceptibility to infections and decreased cancer immunosurveillance. Accordingly, new compounds and compositions that induce tolerance antigen-specific would be beneficial.

Antigen presenting cells, such as dendritic cells, play a key role in regulating the immune response, and, depending on the activation state and the microenvironment of the dendritic cell (cytokines and growth factors), it gives the antigen-specific T cells signal to either combat the presented antigens (presumed pathogens) or to silence the reaction to the presented antigens (presumed non-pathogenic antigens) and induce peripheral tolerance. The challenge in developing tolerogenic immunotherapies is to efficiently deliver the antigen to the APCs/dendritic cells in a manner that does not trigger an inflammatory immune response.

### SUMMARY

The present disclosure relates to tolerance-inducing constructs that comprise an antigen unit and a first and a second targeting unit that interact with surface molecules on antigen-presenting cells, such as dendritic cells, in a non-inflammatory or tolerogenic manner, which leads to the presentation of the antigen in the absence of an inflammatory activation status.

The present inventors have surprisingly found that constructs of the disclosure can deliver disease relevant antigens to the optimal antigen-presenting cells (APCs) in an optimal way for the induction of an antigen-specific tolerogenic response of choice, through binding to and signalling through selected surface receptors on APCs that internalize the construct and present the antigen in a tolerance inducing manner, e.g. induction of regulatory T cells (Tregs) and suppression of memory and effector T cell responses.

Thus, in a first aspect, the disclosure provides a tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
   wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii), such as a dimeric protein consisting of two polypeptides as defined in ii).

In another aspect, the disclosure provides a tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
   wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii).

Thus, in another aspect, the disclosure provides a tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
   wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides as defined in ii).

Also provided herein is a multimeric protein, such as a dimeric protein, as described herein, wherein the multiple polypeptides are linked to each other via their respective first joint regions and via their respective second joint regions.

Also provided herein is a multimeric protein as described herein, wherein the multiple polypeptides, for example two polypeptides, are linked to each other via their respective first joint regions and via their respective second joint regions.

Also provided herein is a dimeric protein as described herein, wherein the two polypeptides are linked to each other via their respective first joint regions and via their respective second joint regions.

In a further aspect the disclosure provides a method of preparing a pharmaceutical composition, said method comprising:
a) providing the polynucleotide, the polypeptide or the multimeric protein, such as the dimeric protein, as described herein; and
b) combining the polynucleotide, the polypeptide or the multimeric protein, such as the dimeric protein, with a pharmaceutically acceptable carrier.

In a further aspect the disclosure provides a method of preparing a pharmaceutical composition, said method comprising:
a) providing the polynucleotide, the polypeptide or the multimeric protein as described herein; and
b) combining the polynucleotide, the polypeptide or the multimeric protein with a pharmaceutically acceptable carrier.

In a further aspect the disclosure provides a method of preparing a pharmaceutical composition, said method comprising:
a) providing the polynucleotide, the polypeptide or the dimeric protein as described herein; and
b) combining the polynucleotide, the polypeptide or the dimeric protein with a pharmaceutically acceptable carrier.

In a further aspect the disclosure provides a pharmaceutical composition comprising the polynucleotide, the polypeptide or the multimeric protein, such as the dimeric protein, as described herein, and a pharmaceutically acceptable carrier.

Also provided herein is a pharmaceutical composition comprising the polynucleotide, the polypeptide or the multimeric protein as described herein, and a pharmaceutically acceptable carrier.

Also provided herein is a pharmaceutical composition comprising the polynucleotide, the polypeptide or the dimeric protein as described herein, and a pharmaceutically acceptable carrier.

In a further aspect the disclosure provides a vector comprising the polynucleotide as described herein.

In a further aspect the disclosure provides a host cell comprising the vector as described herein.

In a further aspect the disclosure provides a method of preparing a polypeptide or a multimeric protein, such as a dimeric protein, said method comprising:
a) transfecting a cell with the vector as described herein or the polynucleotide as described herein;
b) culturing the cell, whereby the cell expresses a polypeptide encoded by said polynucleotide; and
c) obtaining and purifying the multimeric protein, such as the dimeric protein, and/or the polypeptide expressed by the cell.

In a further aspect the disclosure provides a method of preparing a polypeptide or a multimeric protein said method comprising:
a) transfecting a cell with the vector as described herein or the polynucleotide as described herein;
b) culturing the cell, whereby the cell expresses a polypeptide encoded by said polynucleotide; and
c) obtaining and purifying the multimeric protein and/or the polypeptide expressed by the cell.

In a further aspect the disclosure provides a method of preparing a polypeptide or a dimeric protein said method comprising:
a) transfecting a cell with the vector as described herein or the polynucleotide as described herein;
b) culturing the cell, whereby the cell expresses a polypeptide encoded by said polynucleotide; and
c) obtaining and purifying the dimeric protein and/or the polypeptide expressed by the cell.

In a further aspect the disclosure provides a method for treating conditions involving undesired immune reactions, such as in the prophylactic or therapeutic treatment of autoimmune diseases, allergic diseases and graft rejection, said method comprising administering the polynucleotide, the polypeptide or the multimeric protein, such as the dimeric protein, as described herein, the vector as described herein or the pharmaceutical composition as described herein, to a subject in need thereof.

In a further aspect the disclosure provides a method for treating conditions involving undesired immune reactions, such as in the prophylactic or therapeutic treatment of autoimmune diseases, allergic diseases and graft rejection, said method comprising administering the polynucleotide, the polypeptide or the multimeric protein as described herein, the vector as described herein or the pharmaceutical composition as described herein, to a subject in need thereof.

In a further aspect the disclosure provides a method for treating conditions involving undesired immune reactions, such as in the prophylactic or therapeutic treatment of autoimmune diseases, allergic diseases and graft rejection, said method comprising administering the polynucleotide, the polypeptide or the dimeric protein as described herein, the vector as described herein or the pharmaceutical composition as described herein, to a subject in need thereof.

### DESCRIPTION OF DRAWINGS

FIG. 1

Shows a schematic drawing of the immunotherapy construct according to the disclosure.

The figure in the top illustrates an embodiment of the construct as a polypeptide. The figure below shows an embodiment of a dimeric protein formed by two polypeptides linked via their respective first and second joint regions.

A shows a first targeting unit

B shows a second targeting unit

C shows an antigenic unit comprising at least one T cell epitope

D illustrates the flexibility rendered to the targeting unit due to the presence of the

flexible unit

A.A shows a first joint region

B.A shows a second joint region.

FIG. 2

The figure shows an embodiment of the joint region.

A shows three covalent bonds that are formed between the covalent biding units comprised in each of the two polypeptide chains.

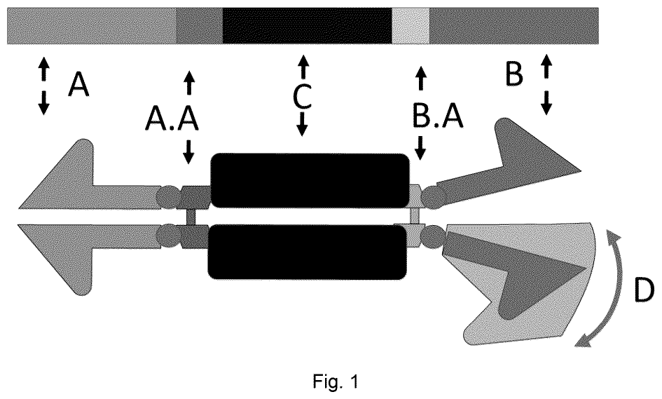

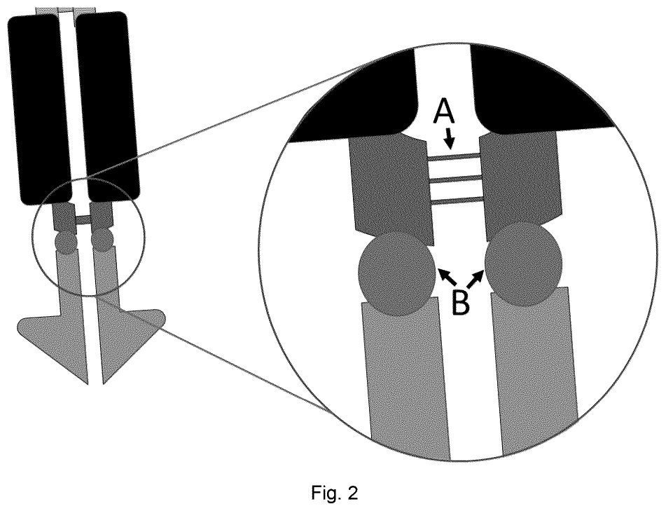

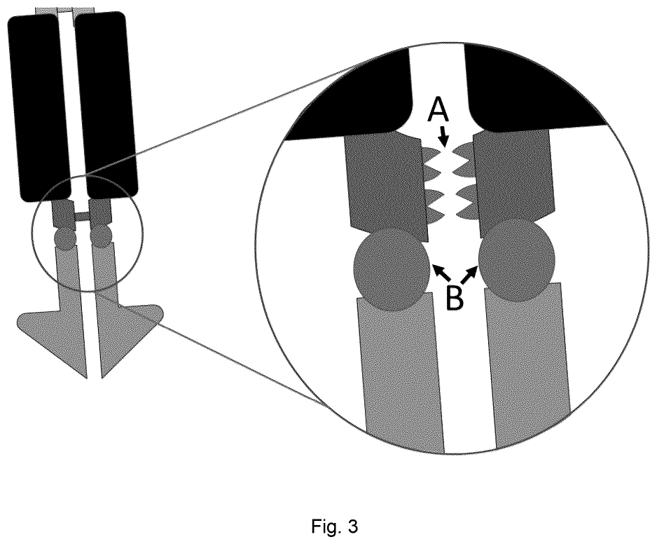

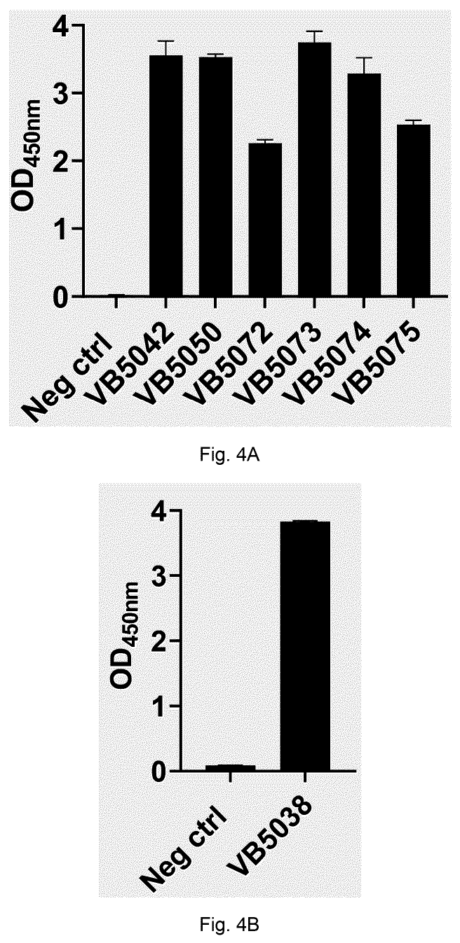

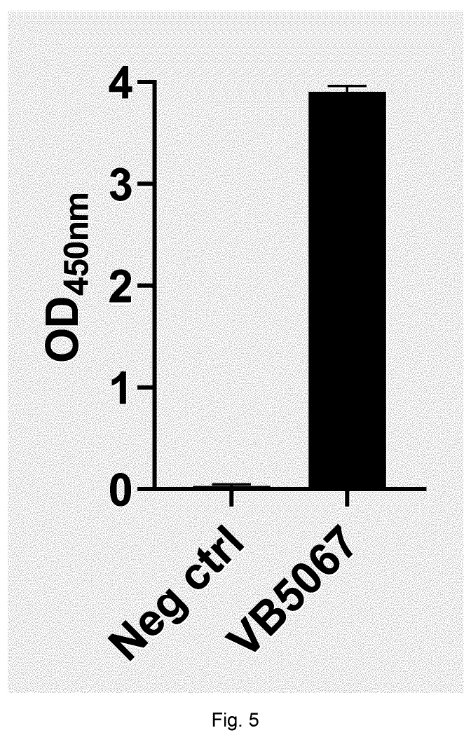

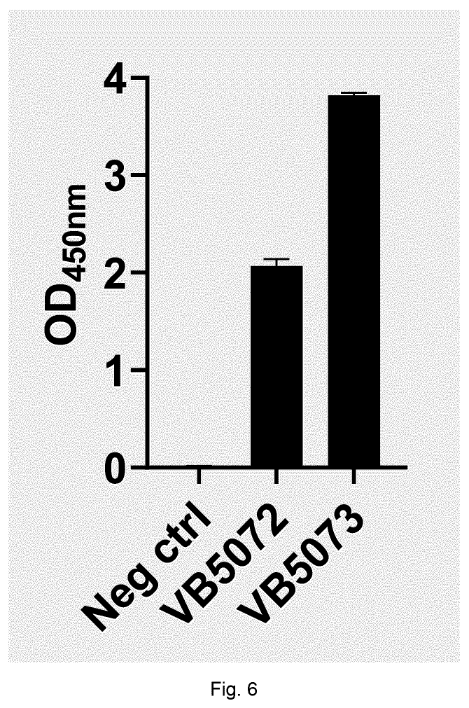

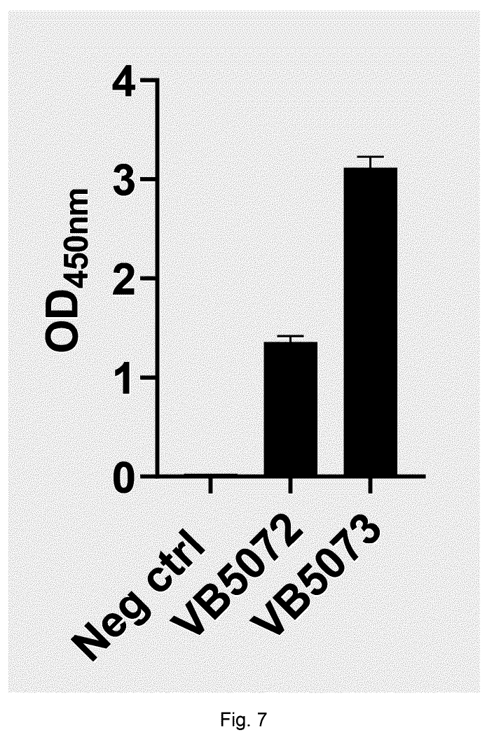

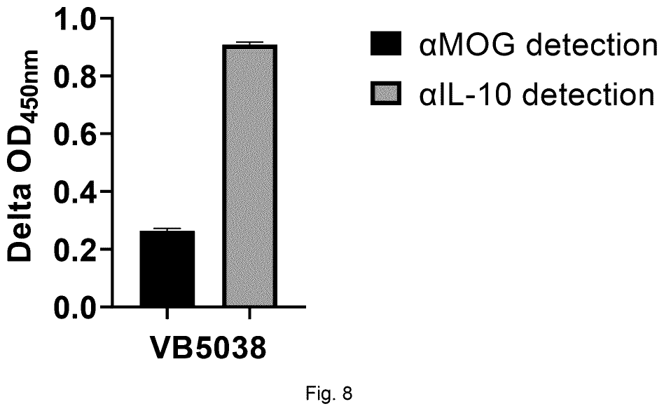

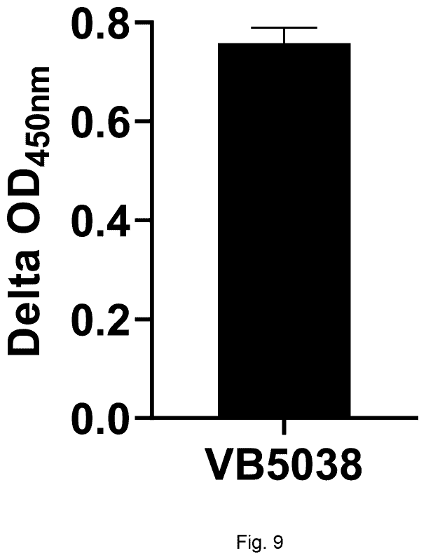

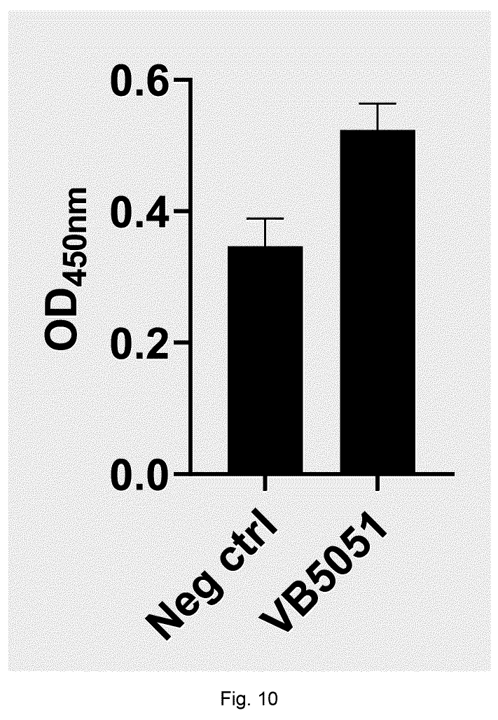

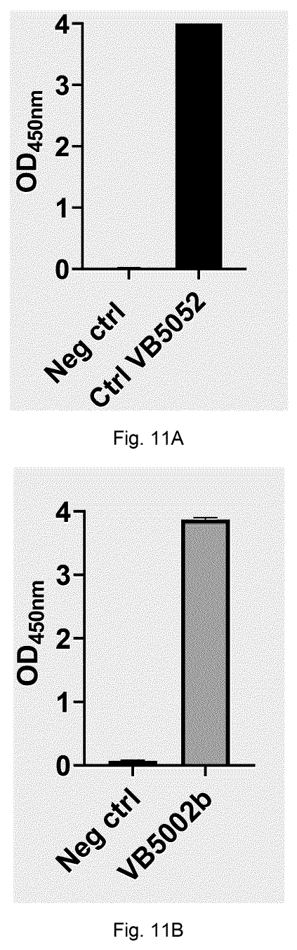

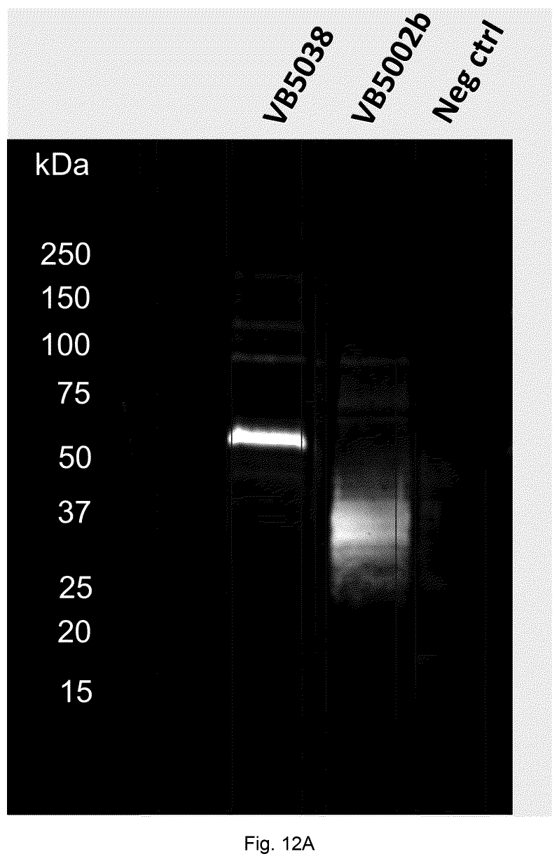

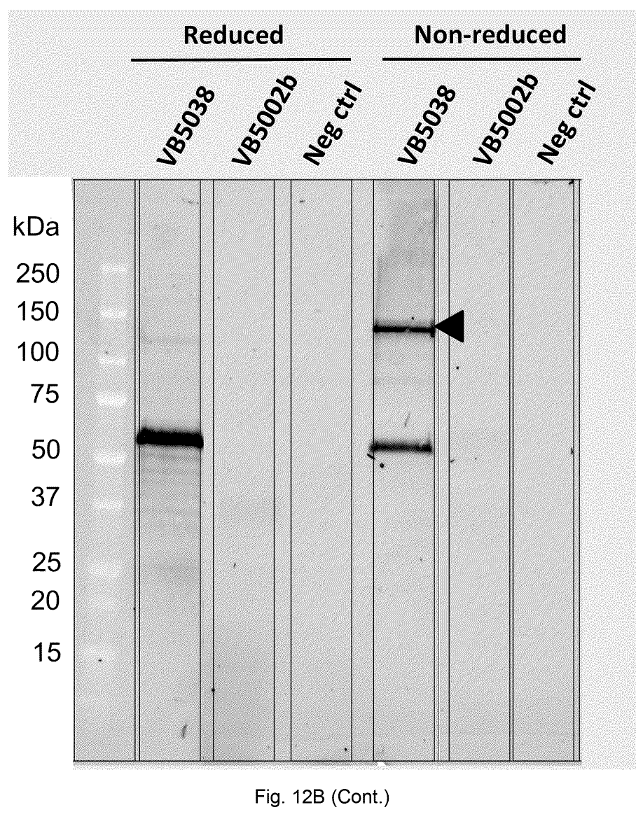

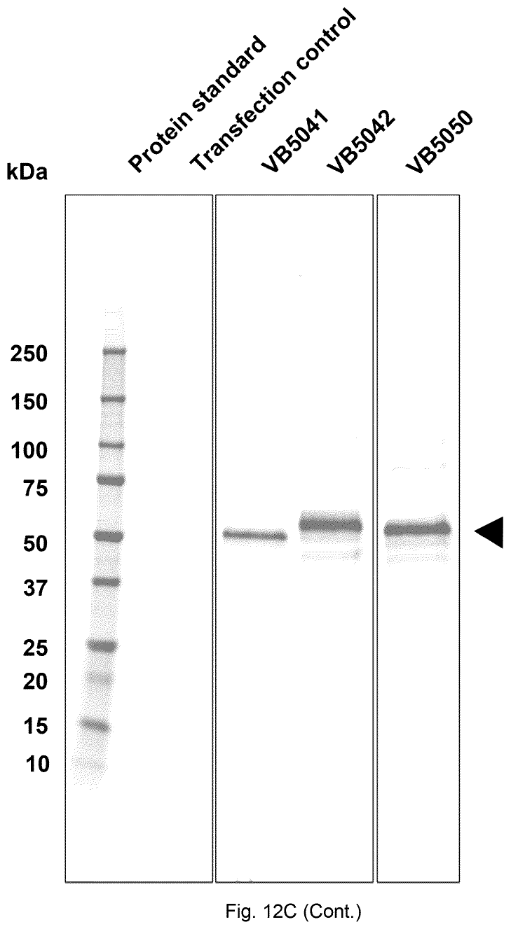

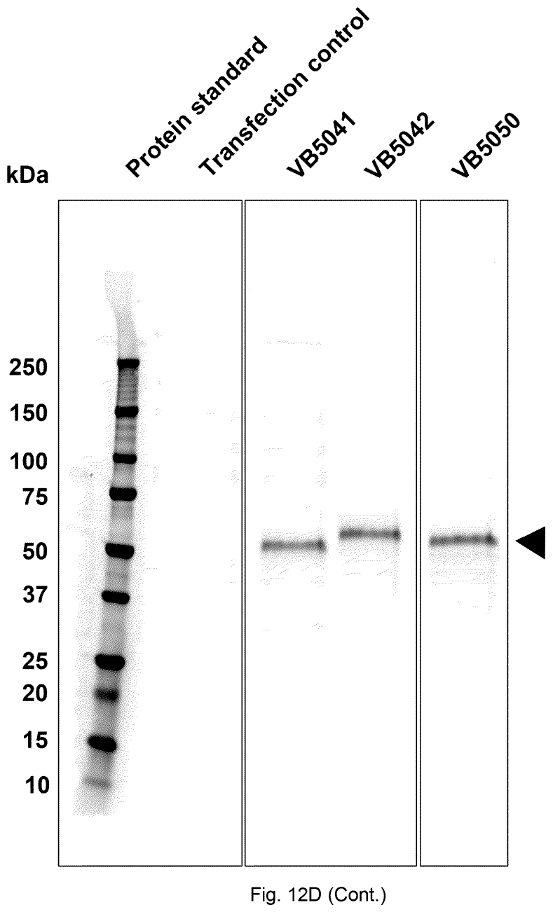

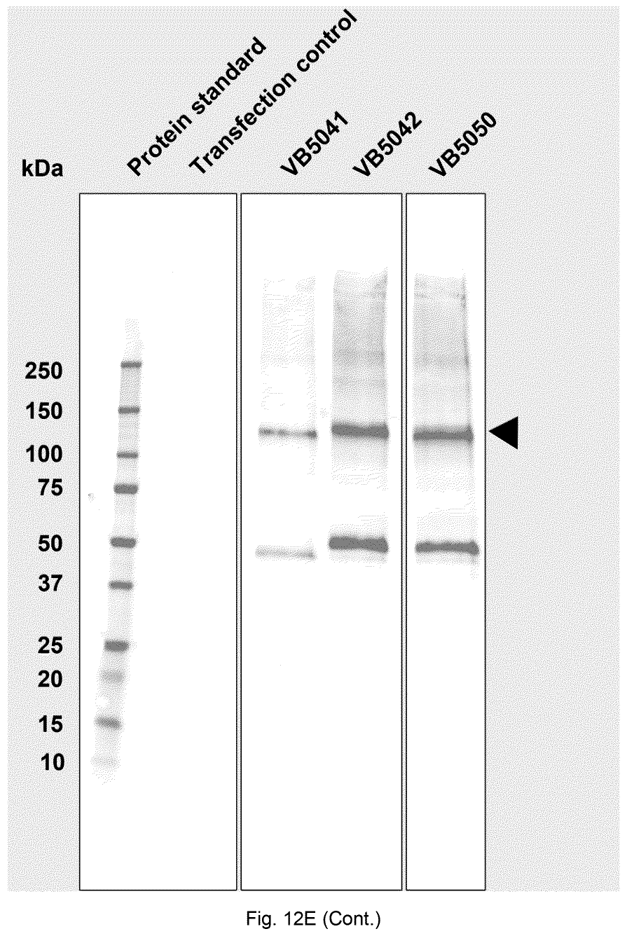

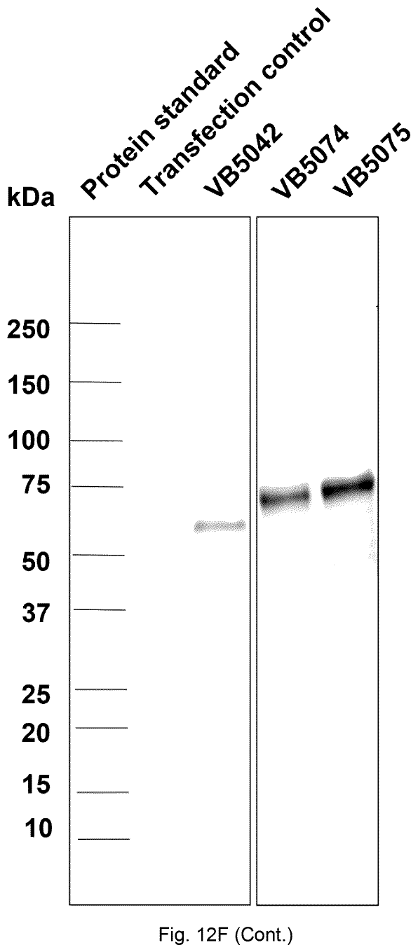

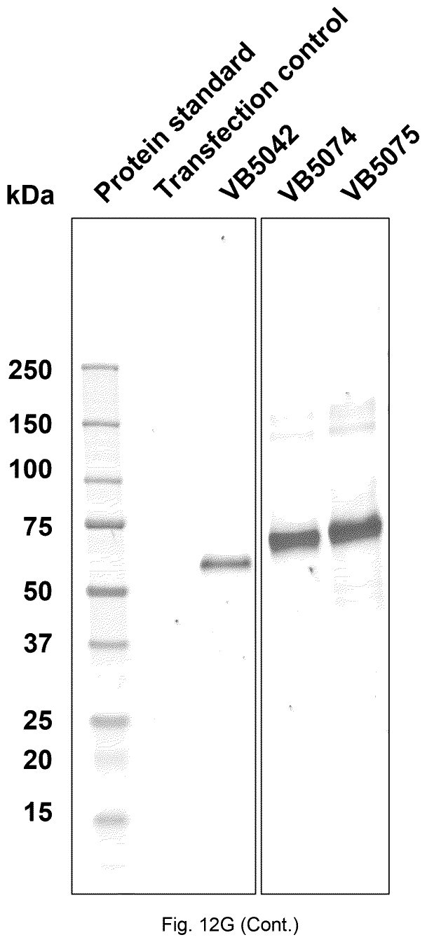

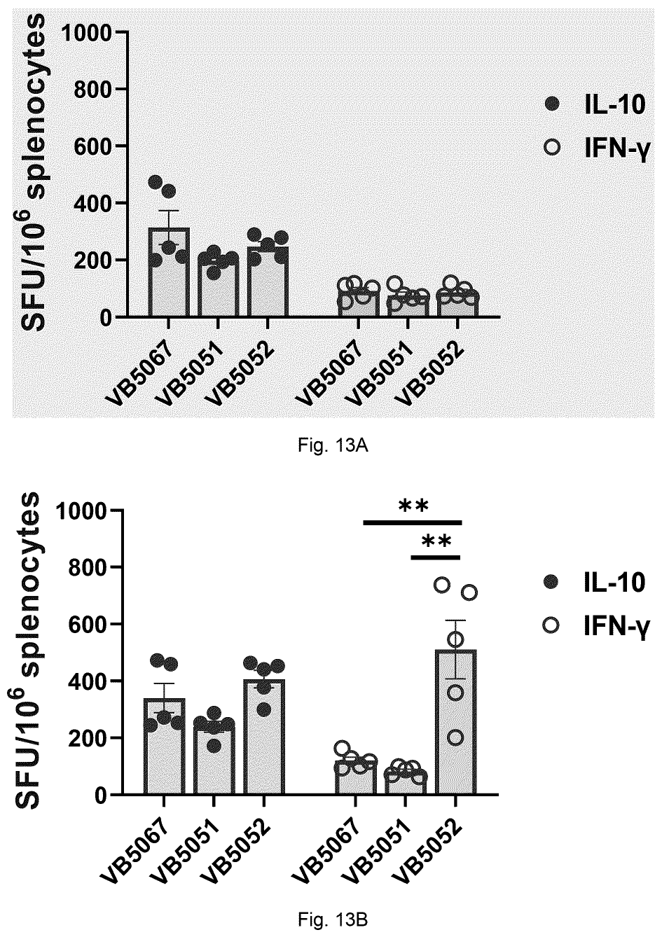

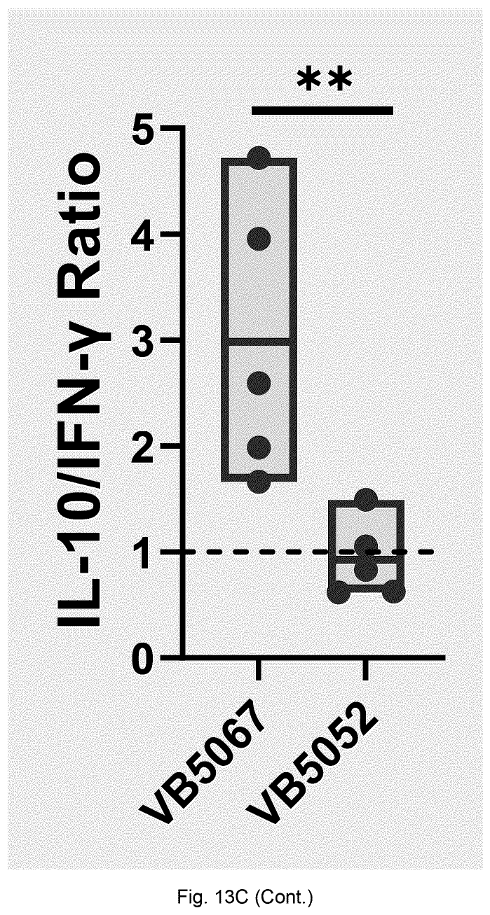

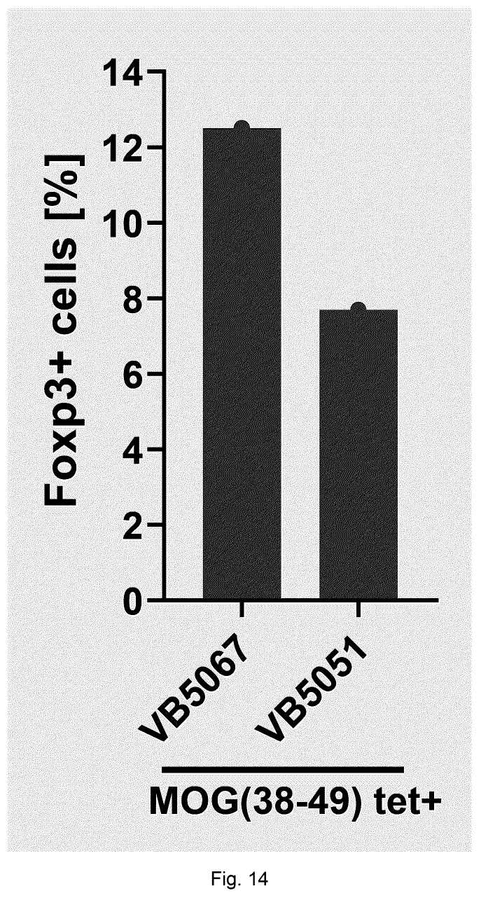

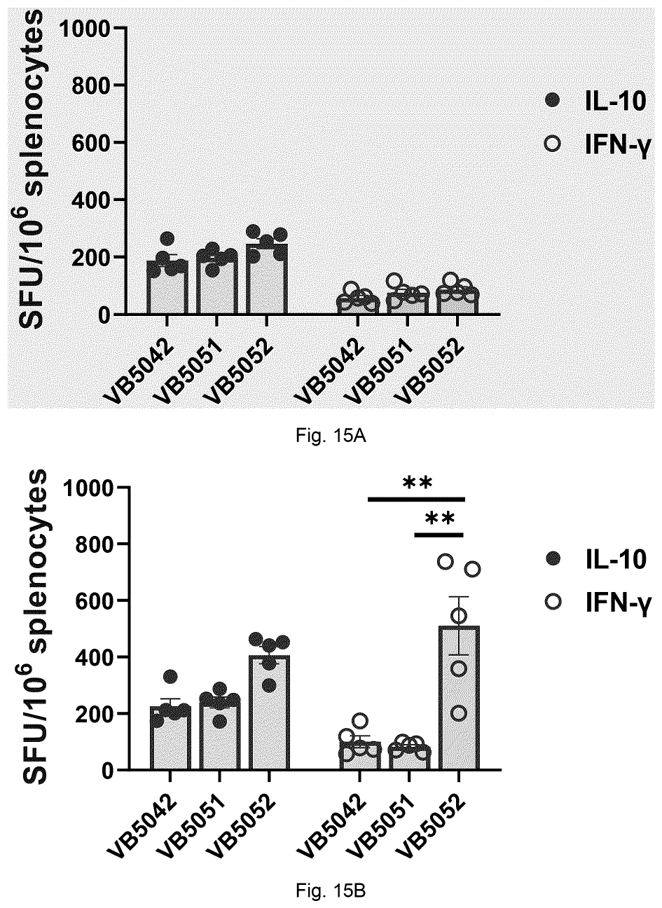

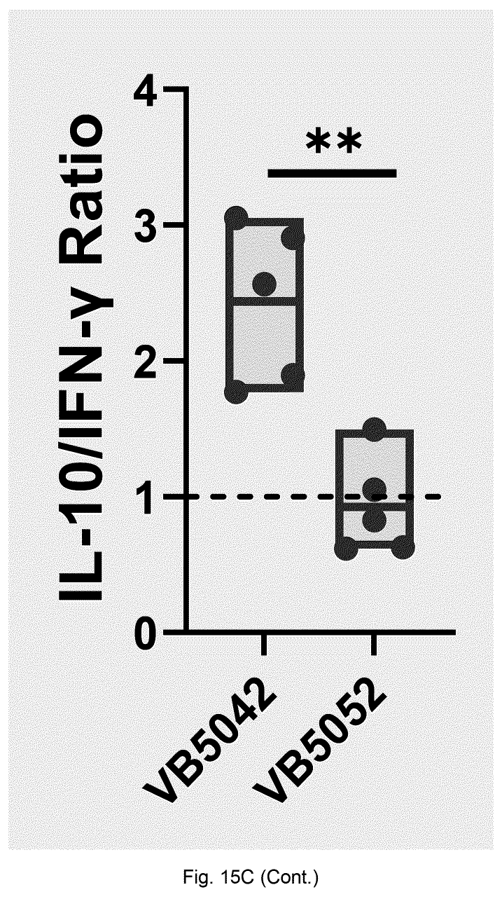

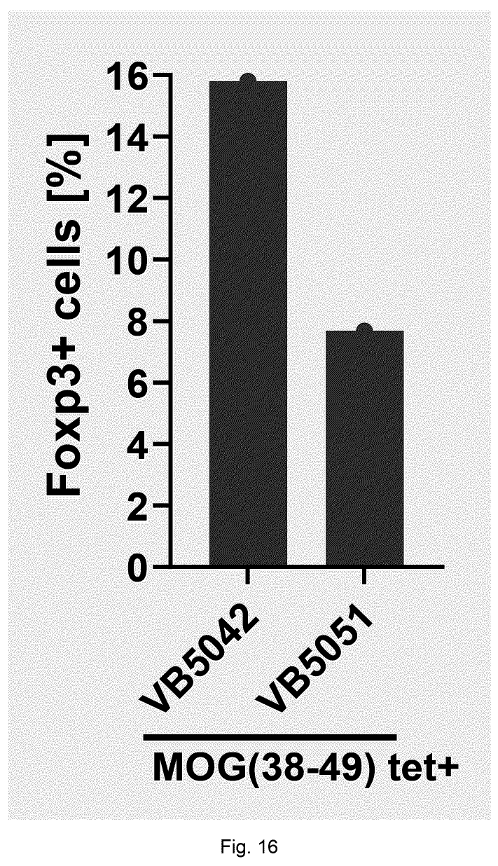

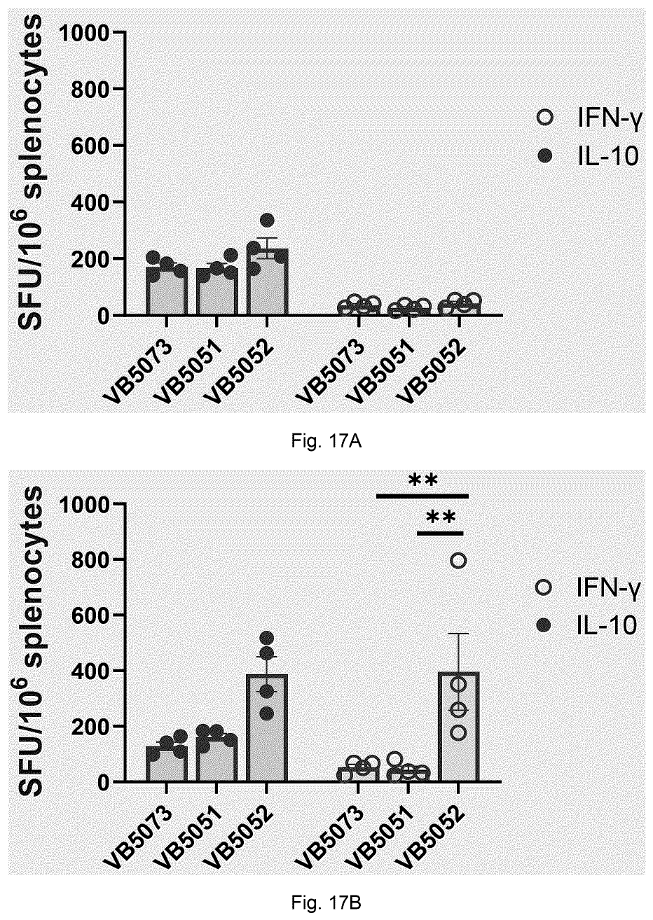

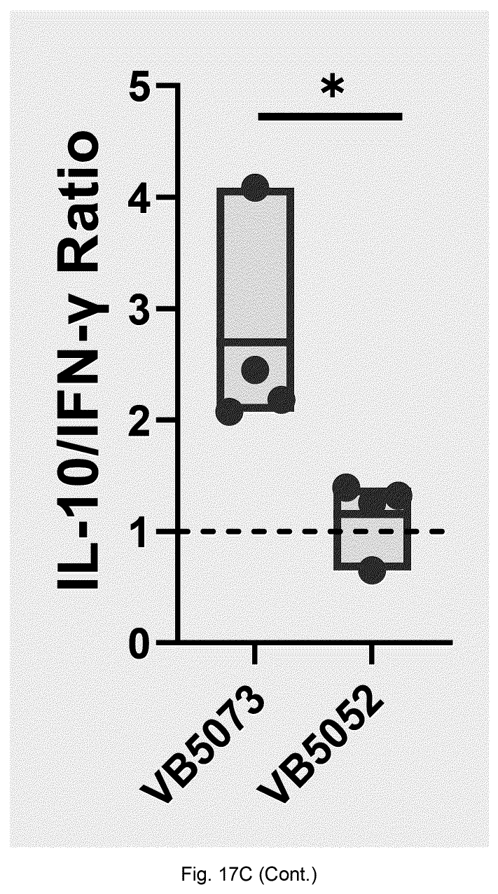

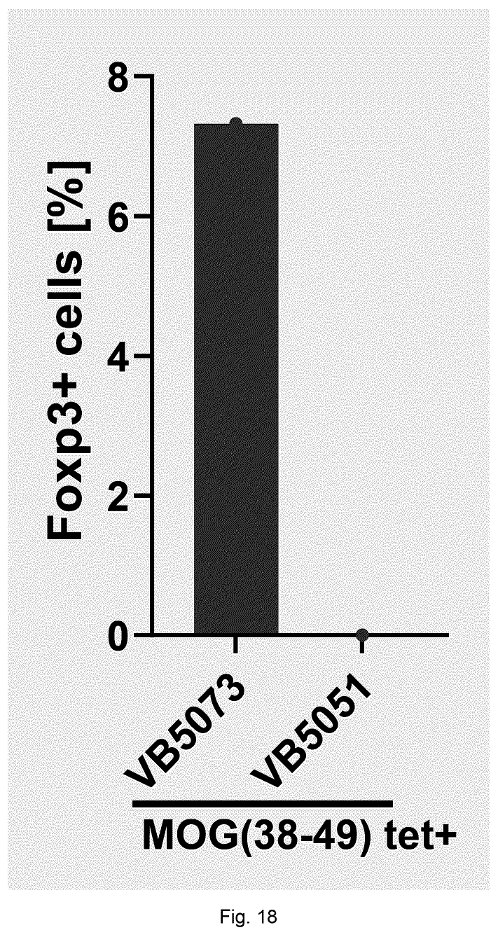

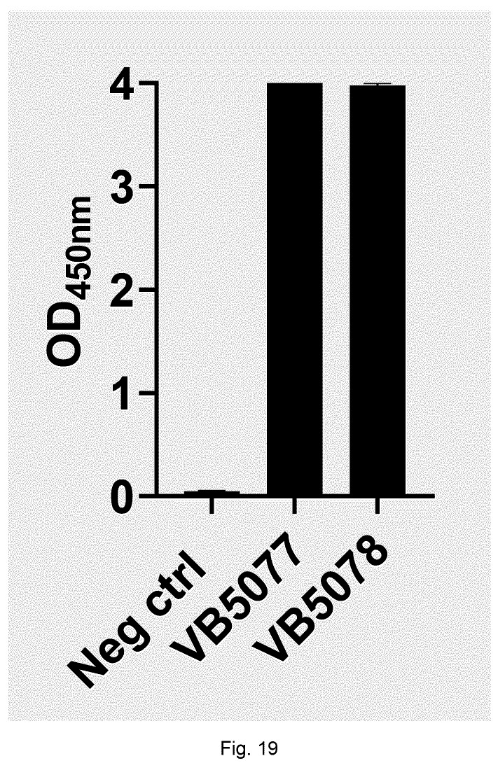

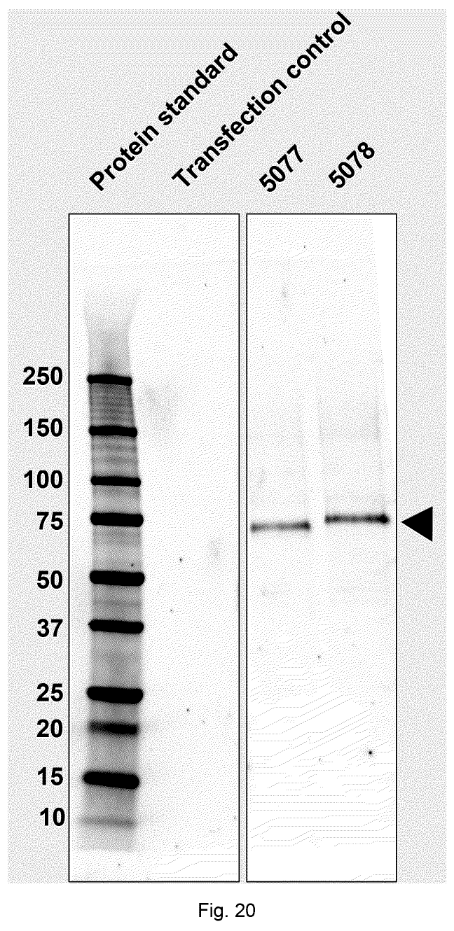

B shows how the flexible unit is located between the binding unit and the targeting unit, providing flexibility to the targeting unit, as shown by arrow D in FIG. 1.

FIG. 3

The figure shows another embodiment of the joint region.

A shows the dimerization of the two polypeptide chains by hydrophobic interactions between the non-covalent binding units comprised in each of the polypeptides.

B shows how the flexible unit is located between the binding unit and the targeting unit, providing flexibility to the targeting, as shown by arrow D in FIG. 1.

FIG. 4

The figure shows expression and secretion levels of MOG and IL-10 encoding tolerance-inducing constructs of the disclosure as detected by sandwich ELISA (capture antibody: mouse anti-MOG antibody, 0.25 μg/ml, 100 μl/well, sc-73330, Santa Cruz Biotechnology, detection antibody: goat anti-murine IL-10 biotinylated antibody, 0.8 μg/ml, 100 μl/well, BAF417, R&D Systems) in supernatant of transfected cells.

A) shows the results from Expi293F cells transiently transfected with the DNA vectors VB5042, VB5050, VB5072, VB5073, VB5074, and VB5075.

B) shows the results from HEK293 cells transiently transfected with the DNA vector VB5038.

All the MOG- and IL-10-encoding constructs were highly expressed and secreted. The negative control in (A) is supernatant form Expi293F cells treated with the transfection reagent ExpiFectamine only and in (B) supernatant from HEK293 cells treated with the transfection reagent Lipofectamine only.

FIG. 5

The figure shows the protein expression and secretion level of the MOG encoding construct with CTLA-4 as second targeting unit (VB5067) as detected by sandwich ELISA (capture antibody: mouse anti-MOG antibody, 0.25 μg/ml, 100 μl/well, sc-73330, Santa Cruz Biotechnology, detection antibody: goat anti-murine CTLA-4 biotinylated antibody, 0.8 μg/ml, 100 μl/well, BAF476, R&D Systems) with supernatant from Expi293F cells transiently transfected with DNA vector VB5067. The negative control is supernatant from Expi293F cells treated with the transfection reagent ExpiFectamine only.

FIG. 6

The figure shows the protein expression and secretion level of the MOG encoding tolerance-inducing constructs with the MARCO ligand SCGB3A2 as first targeting unit and IL-10 as second targeting unit (VB5072 and VB5073) by sandwich ELISA (capture antibody: mouse anti-MOG antibody, 0.25 μg/ml, 100 μl/well, sc-73330, Santa Cruz Biotechnology, detection antibody: goat anti-SCGB3A2 biotinylated antibody, 3.3 μg/ml, 100 μl/well, BAF3465, R&D Systems) with supernatant from Expi293F cells transiently transfected with the DNA vectors VB5072 and VB5073. The negative control is supernatant from Expi293F cells treated with the transfection reagent ExpiFectamine only.

FIG. 7

The figure shows that MOG encoding tolerance-inducing constructs with the MARCO ligand SCGB3A2 as first targeting unit and IL-10 as second targeting unit (VB5072 and VB5073) were secreted as full-length fusion proteins by sandwich ELISA (capture antibody: mouse anti-murine IL-10 antibody, 2 μg/ml, 100 μl/well, MAB417, R&D Systems, detection antibody: goat anti-murine SCGB3A2, 3.3 μg/ml, 100 μl/well, BAF3465, R&D Systems) with supernatant from Expi293F cells transiently transfected with the DNA vectors VB5072 and VB5073. The negative control is supernatant from Expi293F cells treated with the transfection reagent ExpiFectamine only.

FIG. 8

The figure shows binding of a scFv anti-DEC205 encoding construct to the DEC205 receptor, and the secretion of full-length protein, by direct ELISA with supernatant from HEK293 cells transiently transfected with the DNA vector VB5038. The ELISA wells were coated with recombinant DEC205receptor (aa 216-503) and binding was detected by antibodies against MOG or murine IL-10. The OD_{450 nm} signal from the negative control, i.e. supernatant from HEK293 cells treated with the transfection reagent Lipofectamine, was subtracted before graphing.

FIG. 9

The figure shows binding of IL-10 containing construct to the IL-10 receptor by direct ELISA with supernatant from HEK293 cells transiently transfected with the DNA vector VB5038. The ELISA wells were coated with recombinant IL-10 receptor and binding was detected by an antibody against MOG. The OD_{450 nm} signal from the negative control, i.e. supernatant from HEK293 cells treated with the transfection reagent Lipofectamine, was subtracted before graphing.

FIG. 10

The figure shows the secretion of the MOG (27-63) peptide by direct ELISA (detection antibody: mouse anti-MOG antibody, 3.3 μg/ml, 100 μl/well, sc-73330, Santa Cruz Biotechnology) with supernatant from Expi293F cells transiently transfected with the DNA vector VB5051. The negative control is supernatant from Expi293F cells treated with the transfection reagent ExpiFectamine only

FIG. 11

A. shows the expression and secretion level of the pro-inflammatory control construct encoded by the DNA vector VB5052, as detected by sandwich ELISA (capture antibody: mouse anti-MOG antibody, 0.25 μg/ml, 100 μl/well, sc-73330, Santa Cruz Biotechnology, detection antibody: goat anti-human CCL3 Biotin antibody, 0.2 μg/ml, 100 μl/well, BAF270, R&D Systems) with supernatant from Expi293F cells transiently transfected with the CCL3L1 containing vector VB5052. The detection of both the MOG and the CCL3L1 part of the fusion protein indicates full-length secretion of the fusion protein. The negative control is supernatant from Expi293F cells treated with the transfection reagent ExpiFectamine only.

B. shows the expression and secretion level of the pro-inflammatory control construct encoded by the DNA vector VB5002b, as detected by Sandwich ELISA (capture antibody: mouse anti-human IgG (CH3 domain), 1 μg/ml, 100 μl/well, 153272, Biorad, detection antibody: goat anti-human CCL3 biotin antibody, 0.2 μg/ml, 100 μl/well, BAF270, R&D Systems) with supernatant from HEK293 cells transiently transfected with the CCL3L1 containing vector VB5002b. The negative control is supernatant from HEK293 cells treated with the transfection reagent Lipofectamine only.

FIG. 12

A. shows a Western blot with full-length secretion of the tolerance-inducing protein encoded by VB5038 and the pro-inflammatory control encoded by VB5002b. Reduced supernatant samples (10 μL loaded) from transfected Expi293F cells. Primary antibody: mouse anti-MOG (sc-73330). Secondary antibody: donkey anti-mouse, Dylight 800 (SA5-10172). Chemidoc channel Dylight 800.

B. shows a Western blot of the protein encoded by VB5038 under reducing and non-reducing conditions, detected with an anti-murine IL-10 antibody. Reduced supernatant samples are shown to the left and non-reduced supernatant samples are shown to the right (10 μL loaded) from transfected Expi293F cells. Primary antibody: rat anti-murine IL10 (MAB417). Secondary antibody: donkey anti-rat, Dylight 488 (SA5-1006). Chemidoc channel Dylight 488. A specific band was detected at the size corresponding to homodimeric proteins (indicated by black arrow head) under non-reducing conditions.

C. shows a Western blot with full-length secretion of proteins encoded by VB5041, VB5042 and VB5050 as detected by an anti-MOG antibody (black arrowhead). Reduced supernatant samples (30 μL loaded) from transfected Expi293F cells. Primary antibody: mouse anti-MOG (sc-73330). Secondary antibody: donkey anti-mouse, Dylight 800 (SA5-10172). Chemidoc channels Dylight 650 (for protein standard) and 800.

D shows Western blot with full-length secretion of proteins encoded by VB5041, VB5042 and VB5050 as detected by an anti-murine IL-10 antibody (black arrow). Reduced supernatant samples (30 μL loaded) from transfected Expi293F cells. Primary antibody: rat anti-murine IL10 (MAB417). Secondary antibody: donkey anti-rat, Dylight 650 (SA5-10029). Chemidoc channel Dylight 650.

E. shows a Western blot of the proteins encoded by VB5041, VB5042 and VB5050-showing that the proteins dimerize under non-reducing conditions (black arrowhead). Non-reduced supernatant samples (30 μl loaded) from transfected Expi293F cells. Primary antibody: mouse anti-MOG (SC-73330). Secondary antibody: donkey anti-mouse, Dylight 800 (SA5-10172). Chemidoc channels Dylight 650 (for protein standard) and 800.

F. shows a Western blot with full-length secretion of proteins with VSIG-3 as the first targeting unit encoded by VB5074 and VB5075, as detected by an anti-MOG antibody. VB5042 was included as a positive control. Reduced supernatant samples (25 μL loaded) from transfected Expi293F cells. Primary antibody: mouse anti-MOG (sc-73330). Secondary antibody: donkey anti-mouse, Dylight 800 (SA5-10172). Protein standard was detected in Chemidoc channel Dylight 650 (signal not shown). Chemidoc channel Dylight 800.

G. shows a Western blot with full-length secretion of proteins with VSIG-3 as the first targeting unit encoded by VB5074 and VB5075, as detected by an anti-murine IL-10 antibody. VB5042 was included as a positive control. Reduced supernatant samples (25 μL loaded) from transfected Expi293F cells. Primary antibody: Rat anti-IL10 (MAB417). Secondary antibody: donkey anti-rat, Dylight 488 (SA5-10026). Chemidoc channels Dylight 650 (for protein standard) and 488.

FIG. 13

The figure shows dual color IL-10/IFNγ FluoroSpot. C57BL/6 mice were vaccinated once (day 0) with 50 μg of the indicated DNA vectors, and spleens were harvested at day 7 post vaccination. Individual mice and mean±SEM are shown, n=5 mice per group. **(p<0.01), two-tailed Mann-Whitney test. Construct ID numbers are indicated on the x-axis.

A. shows splenocytes of mice tested for IL-10 and IFN-γ secretion (SFU/10⁶ splenocytes) with dual color FluoroSpot for non-stimulated splenocytes.

B. shows splenocytes of mice tested for IL-10 and IFN-γ secretion (SFU/10⁶ splenocytes) with dual color FluoroSpot upon 44 h restimulation with MOG (35-55) peptide.

C. shows the IL-10/IFN-γ ratios are plotted from data in (B) from MOG (35-55)-restimulated splenocytes. Individual mice and mean±range are shown. **(p<0.01), two-tailed Mann-Whitney test.

FIG. 14

The figure shows the detection of MOG (38-49)-specific Foxp3+ cells. C57BL/6 mice were vaccinated once (day 0) with 50 μg of the indicated DNA vectors, and spleens were harvested at day 7 post vaccination. Percentage of splenic CD4+Foxp3+ cells detected by H-2 lab/MOG (38-49) tetramers. The tetramer staining was performed ex vivo and the splenocytes were not restimulated with MOG (35-55) peptide. Data are generated from a pool of 5 mice per group (spleens were pooled before analysis). Construct ID numbers are indicated on the x-axis.

FIG. 15

The figure shows dual color IL-10/IFNγ FluoroSpot. C57BL/6 mice were vaccinated once (day 0) with 50 μg of the indicated DNA vectors, and spleens were harvested at day 7 post vaccination. Individual mice and mean±SEM are shown, n=5 mice per group. **(p<0.01), two-tailed Mann-Whitney test. Construct ID numbers are indicated on the x-axis.

A. shows splenocytes of mice tested for IL-10 and IFN-γ secretion (SFU/10⁶ splenocytes) with dual color FluoroSpot for non-stimulated splenocytes.

B. shows splenocytes of mice tested for IL-10 and IFN-γ secretion (SFU/10⁶ splenocytes) with dual color FluoroSpot upon 44 h restimulation with MOG (35-55) peptide.

C. shows the IL-10/IFN-γ ratios plotted from data in (B) from MOG (35-55)-restimulated splenocytes. Individual mice and mean±range are shown. **(p<0.01), two-tailed Mann-Whitney test.

FIG. 16

The figure shows the detection of MOG (38-49)—specific Foxp3+ cells. C57BL/6 mice were vaccinated once (day 0) with 50 μg of the indicated DNA vectors, and spleens were harvested at day 7 post vaccination. Percentage of splenic CD4+Foxp3+ cells detected by H-2 lab/MOG (38-49) tetramers. The tetramer staining was performed ex vivo and the splenocytes were not restimulated with MOG (35-55) peptide. Data are generated from a pool of 5 mice per group (spleens were pooled before analysis). Construct ID numbers are indicated on the x-axis.

FIG. 17

The figure shows dual color IL-10/IFNγ FluoroSpot. C57BL/6 mice were vaccinated once (day 0) with 50 μg of the indicated DNA vectors, and spleens were harvested at day 7 post vaccination. Individual mice and mean±SEM are shown, n=5 mice per group. **(p<0.01), two-tailed Mann-Whitney test. Construct ID numbers are indicated on the x-axis.

A. shows splenocytes of mice tested for IL-10 and IFN-γ secretion (SFU/10⁶ splenocytes) with dual color FluoroSpot for non-stimulated splenocytes.

B. shows splenocytes of mice tested for IL-10 and IFN-γ secretion (SFU/10⁶ splenocytes) with dual color FluoroSpot upon 44 h restimulation with MOG (35-55) peptide.

C. shows the IL-10/IFN-γ ratios plotted from data in (B) from MOG (35-55)-restimulated splenocytes. Individual mice and mean±range are shown. * (p<0.05), two-tailed Mann-Whitney test.

FIG. 18

The figure shows the detection of MOG (38-49)-specific Foxp3+ cells. C57BL/6 mice were vaccinated once (day 0) with 50 μg of the indicated DNA vectors, and spleens were harvested at day 7 post vaccination. Frequency of splenic CD4+Foxp3+ cells detected by H-2 lab/MOG (38-49) tetramers. The tetramer staining was performed ex vivo and the splenocytes were not restimulated with MOG (35-55) peptide. Data are generated from a pool of 5 mice per group (spleens were pooled before analysis). Construct ID numbers are indicated on the x-axis.

FIG. 19

The figure shows the expression and secretion level of the Met e 1-containing tolerance-inducing constructs with IL-10 as second targeting unit, VB5077 and VB5078. Sandwich ELISA: capture antibody: anti-murine IL-10 antibody (MAB417, R&D Systems), detection antibody: anti-murine IL-10 biotinylated antibody (BAF417, R&D Systems) with supernatant from Expi293F cells transiently transfected with VB5077 and VB5078. Both the Met e 1-containing constructs were highly expressed and secreted. The negative control is supernatant from Expi293F cells treated with the transfection reagent ExpiFectamine only.

FIG. 20

The figure shows a Western blot with full-length secretion of the proteins encoded by the Met e 1-containing DNA vectors VB5077 and VB5078 (black arrowhead). Reduced supernatant samples (35 μL loaded) from transfected Expi293F cells. Primary antibody: Rat anti-IL10 (MAB417). Secondary antibody: Donkey anti-rat, Dylight 650 (SA5-10029). Chemidoc channel Dylight 650.

### DETAILED DESCRIPTION

In a first aspect, the disclosure provides a tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii), such as a dimeric protein consisting of two polypeptides as defined in ii).

In another aspect, the disclosure provides a tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii).

In another aspect, the disclosure provides a tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides as defined in ii).

Such a construct will, once administered to a subject, allow the presentation of the epitopes in the antigenic unit in a tolerance-inducing manner and is thus suitable for use as a prophylactic or therapeutic treatment of immune diseases such as autoimmune diseases, allergic diseases and graft rejection.

As the tolerance-inducing construct causes downregulation of the disease-specific cells of the immune system causing the immune disease in question, it will not suppress the general immune system. Thus, treatment of the immune disease in question with the construct of the disclosure will therefore not result in increased susceptibility to infections and decreased cancer immunosurveillance. However, bystander suppression of immune cells specific for related disease antigens are expected, due to the release of short-range inhibitory cytokines by cell-to-cell contact with the induced antigen-specific regulatory cells.

The tolerance-inducing construct of the disclosure may be administered in the form of a pharmaceutical composition comprising the construct of the disclosure and a pharmaceutically acceptable carrier, for use in the prophylactic or therapeutic treatment of immune disease such as autoimmune diseases, allergic diseases and graft rejection.

A “tolerance-inducing construct” is one that does not elicit an inflammatory immune response but rather does induce tolerance towards the T cell epitopes comprised in the antigenic unit, when administered to a subject in a form suitable for administration and in an amount effective to induce tolerance (i.e. an effective amount).

The term “tolerance” as used herein refers to a decreased level of an inflammatory immune response, a delay in the onset or progression of an inflammatory immune response and/or a reduced risk of the onset or progression of an inflammatory immune response towards harmless antigens like autoantigens, allergens or alloantigens.

A “subject” is an animal, e.g. a mouse, or a human, preferably a human. A subject may be a patient, i.e. a human suffering from an immune disease like an autoimmune disease, an allergy or a graft rejection, who is in need of a therapeutic treatment, or it may be a subject in need of prophylactic treatment or a subject suspected of having an immune disease. The terms “subject” and “individual” are used interchangeably herein.

A “disease” is an abnormal medical condition that is typically associated with specific signs and symptoms in a subject being affected by the disease. An “immune disease” as used herein refers to conditions, disorders or diseases involving undesired immune reactions, including autoimmune diseases, allergies or a graft rejection, i.e. rejection of allografts or xenografts such as rejection by a host of cells, tissue or organs from the same (allo) or a different (xeno) species transplanted to the host.

The term “alloantigen” or “allograft antigen” as used herein refers to an antigen derived from (shed from and/or present in) a cell or tissue which, when transferred from a donor to a recipient, can be recognized and bound by an antibody of B or T-cell receptor of the recipient. Alloantigens are typically products of polymorphic genes. An alloantigen is a protein or peptide which, when compared between donor and recipient (belonging to the same species), displays slight structural differences. The presence of such a donor antigen in the body of a recipient can elicit an inflammatory immune response in the recipient. Such alloreactive immune response is specific for the alloantigen.

The term “xenoantigen” as used herein refers to an antigen derived from an individual of a different species.

A “treatment” is a prophylactic treatment or therapeutic treatment.

A “prophylactic treatment” is a treatment administered to a subject who does not display signs or symptoms of, or displays only early signs or symptoms of, an immune disease, such that treatment is administered for the purpose of preventing or at least decreasing the risk of developing the immune disease. A prophylactic treatment functions as a preventative treatment against an immune disease, or as a treatment that inhibits or reduces further development or enhancement of the immune disease and/or its associated symptoms. The terms “prophylactic treatment”, “prophylaxis” and “prevention” are used interchangeably herein.

A “therapeutic treatment” is a treatment administered to a subject who displays symptoms or signs of an immune disease, in which treatment is administered to the subject for the purpose of diminishing or eliminating those signs or symptoms or for the purpose of delaying or stopping disease progression.

A “part” refers to a part/fragment of an antigen, i.e. part/fragment of the amino acid sequence of an antigen, or the nucleotide sequence encoding same, e.g. an epitope; preferably, the part or fragment of the antigen is immunogenic. These terms will be used throughout interchangeably.

A “T cell epitope” as used herein refers to a single T cell epitope or a part or region of an antigen containing multiple T cell epitopes, e.g. multiple minimal epitopes.

The term “minimal epitope” refers to a subsequence of an epitope predicted to bind to MHC I or MHC II. In other words, the minimal epitope may be immunogenic, i.e. capable of eliciting an immune response. The term minimal epitope thus may refer to short subsequences of an epitope, which are predicted to bind to MHC I or MHC II. A 27-mer epitope may thus encompass several minimal epitopes, which may each have a length shorter than 27 amino acids, and which each are immunogenic. For example, a minimal epitope could consist of the first 14 amino acids of the epitope, provided that it is predicted to bind to MHC I or MHC II, or it could consist of amino acids 9 to 18 of the epitope, or of amino acids 7 to 22, provided that these sequences are predicted to bind to MHC I or MHC II.

A “nucleotide sequence” is a sequence consisting of nucleotides. The terms “nucleotide sequence” and “nucleic acid sequence” are used interchangeably herein.

The term “mouse” and “murine” are used interchangeably herein.

The terms ‘vaccination’ and ‘administration’ are used interchangeably herein.

The section headings used herein are for organizational purposes only and are not to be construed as limiting the subject matter described.

### Tolerance-Inducing Construct

The structure of some embodiments of the construct is illustrated in FIGS. 1-3 on the basis of the polypeptide and a dimeric protein formed by two polypeptides linked via their respective first and second joint regions. The polypeptide (FIG. 1, top) comprises, in the specified order, a first targeting unit (A), a first joint region (A.A), an antigenic unit as describe herein (C), a second joint region (B.A) and the second targeting unit (B). The lower part of FIG. 1 shows how the flexible unit comprised in the second joint region provides flexibility to the second targeting unit (arrow D).

In some embodiments the polypeptide and the multimeric protein, such as a dimeric protein, are formed by multiple polypeptides linked via their respective first and second joint regions. The polypeptide comprises, in the specified order, a first targeting unit, a first joint region, an antigenic unit as described herein, a second joint region and a second targeting unit. The flexible unit comprised in the second joint region provides flexibility to the second targeting unit.

In some embodiments the polypeptide and the multimeric protein are formed by multiple polypeptides linked via their respective first and second joint regions. The polypeptide comprises, in the specified order, a first targeting unit, a first joint region, an antigenic unit as described herein, a second joint region and a second targeting unit. The flexible unit comprised in the second joint region provides flexibility to the second targeting unit.

In some embodiments the polypeptide and the dimeric protein are formed by two polypeptides linked via their respective first and second joint regions. The polypeptide comprises, in the specified order, a first targeting unit, a first joint region, an antigenic unit as described herein, a second joint region and a second targeting unit. The flexible unit comprised in the second joint region provides flexibility to the second targeting unit.

In the following disclosure, the various units of the construct will be discussed in detail. They are comprised in the polynucleotide as nucleic acid sequences encoding the units while they are comprised in the polypeptide or multimeric/dimeric protein as amino acids sequences. For the ease of reading, in the following disclosure, the units of the construct are mainly explained in relation to the polypeptide or multimeric/dimeric protein, i.e. on the basis of their amino acid sequences.

### Joint Regions

The polypeptide of the disclosure comprises a first joint region and a second joint region. The first joint region and the second joint region may be any of the below described regions.

The first joint region is located between the first targeting unit as described herein and the antigenic unit as described herein.

In some embodiments, the second joint region is located between the antigenic unit as described herein and the second targeting unit as described herein.

In some embodiments, the multimeric protein, such as a dimeric protein, of the disclosure is one where the multiple polypeptides, for example the two polypeptides, are linked to each other via their joint regions. In other embodiments, the multimeric protein, such as a dimeric protein, of the disclosure is one where the multiple polypeptides, such as the two polypeptides, are linked to each other via their respective first joint regions and via their respective second joint regions.

In some embodiments, the multimeric protein of the disclosure is one where the multiple polypeptides are linked to each other via their joint regions. In other embodiments, the multimeric protein of the disclosure is one where the multiple polypeptides are linked to each other via their respective first joint regions and via their respective second joint regions.

In some embodiments, the dimeric protein of the disclosure is one where the two polypeptides are linked to each other via their joint regions. In other embodiments, the dimeric protein of the disclosure is one where the two polypeptides are linked to each other via their respective first joint regions and via their respective second joint regions. The term “joint region” as used herein refers to a sequence of amino acids between the antigenic unit and the targeting unit. Any amino acid sequence that is capable of joining the multiple polypeptides (for embodiments relating to a multimeric protein), for example capable of joining the two polypeptides (for embodiments relating to a dimeric protein) but at the same time providing flexibility and appropriate protein conformation to the multimeric or dimeric protein is a suitable joint region.

The joint regions provide flexibility to the multimeric protein, such as the dimeric protein, such that targeting units can interact with surface molecules on APCs, e.g. with surface molecules on the same APC, even if they are located at variable distances. In addition, the joint regions join the multiple monomeric polypeptides into a multimeric protein, such as a dimeric protein. Any amino acid sequence that fulfils one or more of these requirements is a suitable joint region.

The joint regions provide flexibility to the multimeric protein, such that targeting units can interact with surface molecules on APCs, e.g. with surface molecules on the same APC, even if they are located at variable distances. In addition, the joint regions join the multiple monomeric polypeptides into a multimeric protein. Any amino acid sequence that fulfils one or more of these requirements is a suitable joint region.

The joint regions provide flexibility to the dimeric protein, such that targeting units can interact with surface molecules on APCs, e.g. with surface molecules on the same

APC, even if they are located at variable distances. In addition, the joint regions join the two monomeric polypeptides into a dimeric protein. Any amino acid sequence that fulfils one or more of these requirements is a suitable joint region.

Preferably, the joint regions comprise a flexible unit which provides flexibility and a binding unit which joins the multiple polypeptides, such as the two polypeptides, to form a multimer, such as a dimer. In preferred embodiments, the flexible unit comprised in the joint region is closest to the targeting unit and the binding unit is closest to the antigenic unit. In other embodiments, the joint region comprises a flexible unit which provides flexibility and a binding unit which joins the multiple polypeptides to form a multimeric protein.

Preferably, the joint regions comprise a flexible unit which provides flexibility and a binding unit which joins the multiple polypeptides to form a multimer. In preferred embodiments, the flexible unit comprised in the joint region is closest to the targeting unit and the binding unit is closest to the antigenic unit. In other embodiments, the joint region comprises a flexible unit which provides flexibility and a binding unit which joins the multiple polypeptides to form a multimeric protein. Preferably, the joint regions comprise a flexible unit which provides flexibility and a binding unit which joins the two polypeptides to form a dimer. In preferred embodiments, the flexible unit comprised in the joint region is closest to the targeting unit and the binding unit is closest to the antigenic unit. In other embodiments, the joint region comprises a flexible unit which provides flexibility and a binding unit which joins the two polypeptides to form a dimeric protein. In some embodiments the binding units of the first and second joint regions are different.

In some embodiments, the binding unit comprised in the first joint region of one polypeptide molecule is able to bind to the binding unit comprised in the first joint region of another polypeptide molecule, whereby the multiple molecules are linked via their respective first joint regions. Likewise, the binding unit comprised in the second joint region of one polypeptide molecule is able to bind to the binding unit comprised in the second joint region of another polypeptide molecule, whereby the multiple molecules are linked via their respective second joint regions. Thus, the multiple polypeptide molecules form a multimeric protein, such as a dimeric protein, by being linked to each other via their respective first joint regions and via their respective second joint regions.

In some embodiments, the binding unit comprised in the first joint region of one polypeptide molecule is able to bind to the binding unit comprised in the first joint region of another polypeptide molecule, whereby the multiple molecules are linked via their respective first joint regions. Likewise, the binding unit comprised in the second joint region of one polypeptide molecule is able to bind to the binding unit comprised in the second joint region of another polypeptide molecule, whereby the multiple molecules are linked via their respective second joint regions. Thus, the multiple polypeptide molecules form a multimeric protein by being linked to each other via their respective first joint regions and via their respective second joint regions.

Thus, in some embodiments, the binding unit comprised in the first joint region of one polypeptide molecule is able to bind to the binding unit comprised in the first joint region of another polypeptide molecule, whereby the two molecules are linked via their respective first joint regions. Likewise, the binding unit comprised in the second joint region of one polypeptide molecule is able to bind to the binding unit comprised in the second joint region of another polypeptide molecule, whereby the two molecules are linked via their respective second joint regions. Thus, the two polypeptide molecules form a dimeric protein by being linked to each other via their respective first joint regions and via their respective second joint regions.

In other embodiments, the first joint region and the second joint region are the same.

Thus, in some embodiments, the binding unit comprised in the first joint region of one polypeptide molecule is able to bind to the binding unit comprised in either the first joint region or the second joint region of another polypeptide molecule. The same applies to the binding unit comprised in the second joint region.

It is preferred that if the first and second joint regions are the same, the first and second targeting units either are different, but interact with the same surface molecule on the APCs; or the first and second targeting units are identical.

In some embodiments, the amino acid sequence of the first and/or second joint region comprises at least one naturally occurring sequence or consists of a naturally occurring sequence. In some embodiments, the amino acid sequence of the first and/or second joint region comprises at least one artificial sequence or consists of an artificial sequence.

In some embodiments, the binding unit is a covalent binding unit, in other embodiments, the binding unit is a non-covalent binding unit.

In preferred embodiments, the amino acid sequence of the joint region is a non-immunogenic sequence.

Embodiments of the joint region comprised in some embodiments of a dimeric protein are illustrated in FIGS. 2 and 3.

The joint region illustrated in FIG. 2 (joint region 1 or joint region 2) comprises a flexible unit (B) closest to the targeting unit and a covalent binding unit adjacent to it, which is closest to the antigenic unit. The covalent binding unit of FIG. 2 shows three covalent bonds (A) that are formed between the two polypeptide chains

The joint region illustrated in FIG. 3 (joint region 1 or joint region 2) comprises a flexible unit (B) closest to the targeting unit and a non-covalent binding unit adjacent to it, which is closest to the antigenic unit. The non-covalent binding unit of FIG. 3 facilitates the dimerization of the two polypeptide chains by for example hydrophobic interactions (A).

### Flexible Unit

In some embodiments, the joint region as described herein comprises a flexible unit.

In preferred embodiments, the amino acid sequence of the flexible unit is a non-immunogenic sequence.

In some embodiments, the amino acid sequence of the flexible unit is a naturally occurring peptide sequence. In some embodiments, the flexible unit is derived from an immunoglobulin. In some embodiments, the flexible unit is a hinge region of an immunoglobulin, wherein the hinge region does not comprise cysteine residues.

In some embodiments, the amino acid sequence of the flexible unit is an artificial sequence.

In some embodiments, the flexible unit comprises small, non-polar (e.g. glycine, alanine or leucine) or polar (e.g. serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens. In some embodiments, the flexible unit is an artificial sequence, e.g. a serine(S) and/or glycine (G) rich linker, i.e. a linker comprising several serine and/or several glycine residues. Preferred examples are GGGGSGGGSS (SEQ ID NO: 75), GGGSG (SEQ ID NO: 76), GGSGG (SEQ ID NO: 77), SGSSGS (SEQ ID NO: 78), GGGGS (SEQ ID NO: 79) or multiple variants thereof such as GGGGSGGGGS (SEQ ID NO: 80), (GGGGS)m (SEQ ID NO: 81), (GGGSS)m (SEQ ID NO: 82), (GGGSG)m (SEQ ID NO: 83) or (SGSSGS)m (SEQ ID NO: 84), where m is an integer from 1 to 5, e.g., 1, 2, 3, 4, or 5. In preferred embodiments, m is 2. In other preferred embodiments, the serine and/or glycine rich linker further comprises at least one leucine (L) residue, such as at least 1 or at least 2 or at least 3 leucine residues, e.g. 1, 2, 3 or 4 leucine residues.

In some embodiments, the flexible unit comprises or consists of LGGGS (SEQ ID NO: 85), GLGGS (SEQ ID NO: 86), GGLGS (SEQ ID NO: 87), GGGLS (SEQ ID NO: 88) or GGGGL (SEQ ID NO: 89). In other embodiments, the flexible unit comprises or consists of LGGSG (SEQ ID NO: 90), GLGSG (SEQ ID NO: 91), GGLSG (SEQ ID NO: 92), GGGLG (SEQ ID NO: 93) or GGGSL (SEQ ID NO: 94). In yet other embodiments, the flexible unit comprises or consists of LGGSS (SEQ ID NO: 95), GLGSS (SEQ ID NO: 96), or GGLSS (SEQ ID NO: 97).

In other embodiments, the flexible unit comprises or consists of LGLGS (SEQ ID NO: 98), GLGLS (SEQ ID NO: 99), GLLGS (SEQ ID NO: 100), LGGLS (SEQ ID NO: 101) or GLGGL (SEQ ID NO: 102). In other embodiments, the flexible unit comprises or consists of LGLSG (SEQ ID NO: 103), GLLSG (SEQ ID NO: 104), GGLSL (SEQ ID NO: 105), GGLLG (SEQ ID NO: 106) or GLGSL (SEQ ID NO: 107). In other embodiments, the flexible unit comprises or consists of LGLSS (SEQ ID NO: 108), or GGLLS (SEQ ID NO: 109).

In other embodiments, the flexible unit is a serine-glycine linker that has a length of 10 amino acids and comprises 1 or 2 leucine residues.

In some embodiments, the flexible unit comprises or consists of LGGGSGGGGS (SEQ ID NO: 110), GLGGSGGGGS (SEQ ID NO: 111), GGLGSGGGGS (SEQ ID NO: 112), GGGLSGGGGS (SEQ ID NO: 113) or GGGGLGGGGS (SEQ ID NO: 114). In other embodiments, the flexible unit comprises or consists of LGGSGGGGSG (SEQ ID NO: 115), GLGSGGGGSG (SEQ ID NO: 116), GGLSGGGGSG (SEQ ID NO: 117), GGGLGGGGSG (SEQ ID NO: 118) or GGGSLGGGSG (SEQ ID NO: 119). In other embodiments, the flexible unit comprises or consists of LGGSSGGGSS (SEQ ID NO: 120), GLGSSGGGSS (SEQ ID NO: 121), GGLSSGGGSS (SEQ ID NO: 122), GGGLSGGGSS (SEQ ID NO: 123) or GGGSLGGGSS (SEQ ID NO: 124).

In further embodiments, the flexible unit comprises or consists of LGGGSLGGGS (SEQ ID NO: 125), GLGGSGLGGS (SEQ ID NO: 126), GGLGSGGLGS (SEQ ID NO: 127), GGGLSGGGLS (SEQ ID NO: 128) or GGGGLGGGGL (SEQ ID NO: 129). In other embodiments, the flexible unit comprises or consists of LGGSGLGGSG (SEQ ID NO: 130), GLGSGGLGSG (SEQ ID NO: 131), GGLSGGGLSG (SEQ ID NO: 132), GGGLGGGGLG (SEQ ID NO: 133) or GGGSLGGGSL (SEQ ID NO: 134). In other embodiments, the flexible unit comprises or consists of LGGSSLGGSS (SEQ ID NO: 135), GLGSSGLGSS (SEQ ID NO: 136), or GGLSSGGLSS (SEQ ID NO: 137).

In other embodiments, the flexible unit comprises or consists of GSGGGA (SEQ ID NO: 138), GSGGGAGSGGGA (SEQ ID NO: 139), GSGGGAGSGGGAGSGGGA (SEQ ID NO: 140), GSGGGAGSGGGAGSGGGAGSGGGA (SEQ ID NO: 141) or GENLYFQSGG (SEQ ID NO: 142). In yet other embodiments, the flexible unit comprises or consists of SGGGSSGGGS (SEQ ID NO: 143), SSGGGSSGGG (SEQ ID NO: 144), GGSGGGGSGG (SEQ ID NO: 145), GSGSGSGSGS (SEQID NO: 146), GGGSSGGGSG (SEQ ID NO: 147, and amino acids 121-130 of SEQ ID NO: 1), GGGSSS (SEQ ID NO: 148), GGGSSGGGSSGGGSS (SEQ ID NO: 149) or GLGGLAAA (SEQ ID NO: 150).

In other embodiments, the flexible unit comprises or consists of the sequence TQKSLSLSPGKGLGGL (SEQ ID NO: 151). In other embodiments, the flexible unit comprises or consists of the sequence SLSLSPGKGLGGL (SEQ ID NO: 152). In other embodiments, the T cell epitope linker comprises or consists of AAY or GPGPG (SEQ ID NO: 153).

In other embodiments, the flexible unit comprises or consists of a GSAT linker, i.e. a linker comprising one or more glycine, serine, alanine and threonine residues, e.g. a linker comprising or consisting of the sequence GGSAGGSGSGSSGGSSGASGTGTAGGTGSGSGTGSG (SEQ ID NO: 154) or a SEG linker, i.e. a linker comprising one or more serine, glutamic acid and glycine residues, e.g. a linker comprising or consisting of the sequence GGSGGGSEGGGSEGGGSEGGGSEGGGSEGGGSGGGS (SEQ ID NO: 155) or ELKTPLGDTTHT (SEQ ID NO: 19).

In some embodiments, the flexible unit is not a target of proteases.

In some embodiments, the flexible unit consists of up to 20 amino acids, such as at up to 15 amino acids, such as 14 amino acids, such as 13 amino acids, such as 12 amino acids, such as 11 amino acids or 10 amino acids.

In some embodiments, the flexible unit comprises or consists of an amino acid sequence having at least 50% sequence identity to the amino acid sequence 1-12 of SEQ ID NO: 1, such as 60% or such as 70% or such as 80% or such as 90% sequence identity.

In preferred embodiments, the flexible unit is hinge exon h1 of IgG3.

In preferred embodiments, the flexible unit comprises or consists of the amino acid sequence 1-12 of SEQ ID NO: 1.

In some embodiments, the flexible unit comprises or consists of an amino acid sequence having at least 50% sequence identity to the an amino acid sequence 16-23 of SEQ ID NO: 2, such as 60% or such as 70% or such as 80% or such as 90% sequence identity.

In some embodiments, the flexible unit comprises or consists of the amino acid sequence 16-23 of SEQ ID NO: 2, wherein any one of the amino acids of the flexible unit has been substituted, deleted, or inserted for another amino acid, with the proviso that no more than 5 amino acids have been so substituted, deleted, or inserted, such as no more than 4 amino acids, such as no more than 3 amino acids, such as no more than 2 amino acids or no more than 1 amino acid.

In other embodiments, the flexible unit is the lower hinge region of IgG1.

In preferred embodiments, the flexible unit comprises or consists of the amino acid sequence 16-23 of SEQ ID NO: 2.

### Covalent Binding Unit

In some embodiments, the joint region as described herein comprises a covalent binding unit.

In preferred embodiments, the covalent binding unit comprises one or more cysteine residues, and the polypeptides described herein are linked via one or more disulfide bonds formed between the cysteine residue(s) comprised in the covalent binding units of the respective first and second joint regions.

In some embodiments, the covalent binding unit consists of or comprises a cysteine rich sequence.

In some embodiments, the covalent binding unit comprises at least 2 cysteine residues, such as at least 3, 4, 5, 6, 7, 8, 9, 10, 11, 12 or 13 cysteine residues.

In some embodiments, the covalent binding unit of the first joint region comprises a different number of cysteine residues than the covalent binding unit of the second joint region.

In some embodiments, the cysteine residues of the covalent binding unit of first joint region are positioned differently than the cysteine residues of the covalent binding unit of the second joint region. For example, the number of amino acid residues between the cysteine residues of the covalent binding unit of the first joint region is different than that of the second joint region.

In some embodiments, the number of cysteine residues is based on the length of the antigenic unit: the more amino acid residues comprised in the antigenic unit, the higher the number of cysteine residues in the covalent binding unit.

In some embodiments, the covalent binding unit comprises the sequence EPKSCDTPPPCPRCP (SEQ ID NO: 156; corresponding to amino acids 13-27 of SEQ ID NO: 1).

In preferred embodiments, the amino acid sequence of the covalent binding unit is a non-immunogenic sequence.

In some embodiments, the amino acid sequence of the covalent binding unit is an artificial sequence.

In some embodiments, the amino acid sequence of the covalent binding unit is a naturally occurring peptide sequence.

In some embodiments, the covalent binding unit consists of from 2 to 100 amino acids, such as 3 to 70 amino acids, such as 4 to 50 amino acids or 5 to 30 amino acids. In further embodiments, the covalent binding unit consists of 10, 11, 12, 13, 14, 15, 16, 17, 18, 19 or 20 amino acids. In preferred embodiments, the covalent binding unit consists of 15 amino acids. In more preferred embodiments, the covalent binding unit consists of 15 amino acids, whereof 3 are cysteine residues.

In some embodiments, the covalent binding unit is derived from an immunoglobulin.

In some embodiments, the covalent binding unit is a hinge region derived from an immunoglobulin, such as exon h4 of IgG3 or the middle hinge region of IgG1. The hinge region may be Ig derived, such as derived from IgG, e.g. IgG2 or IgG3. In some embodiments, the hinge region is derived from IgM, e.g. comprising or consisting of the nucleotide sequence with SEQ ID NO: 157 or an amino acid sequence encoded by said nucleotide sequence.

In some embodiments, the covalent binding unit comprises or consists of an amino acid sequence having at least 40% sequence identity to the amino acid sequence 13-27 of SEQ ID NO: 1, such as at least 50%, at least 60%, at least 70%, at least 80% or at least 90% sequence identity.

In some embodiments, the covalent binding unit comprises or consists of the amino acid sequence 13-27 of SEQ ID NO: 1, wherein any one of the amino acids of the flexible unit has been substituted, deleted, or inserted for another amino acid, with the proviso that no more than 6 amino acids have been so substituted, deleted, or inserted, such as no more than 5 amino acids, such as no more than 4 amino acids, such as no more than 3 amino acids, such as no more than 2 amino acids or no more than 1 amino acid.

In preferred embodiments, the covalent binding unit is hinge exon h4 of IgG3.

In other preferred embodiments, the covalent binding region consists of amino acid sequence 13-27 of SEQ ID NO: 1.

In some embodiments, the covalent binding unit comprises or consists of an amino acid sequence having at least 40% sequence identity to the amino acid sequence 5-15 of SEQ ID NO: 2, provided that the cysteine residues are retained in their number and position, such as at least 50%, at least 60%, at least 70%, at least 80% or at least 90% sequence identity.

In some embodiments, the covalent binding unit comprises or consists of the amino acid sequence 5-15 of SEQ ID NO: 2, wherein any one of the amino acids of the flexible unit has been substituted, deleted, or inserted for another amino acid, with the proviso that no more than 5 amino acids have been so substituted, deleted, or inserted, such as no more than 4 amino acids, such as no more than 3 amino acids, such as no more than 2 amino acids or no more than 1 amino acid.

In preferred embodiments, the covalent binding unit is the middle hinge region of IgG1.

In other embodiments, the covalent binding unit consists of or comprises the amino acid sequence 5-15 of SEQ ID NO: 2.

### Non-Covalent Binding Unit

In some embodiments, the joint region as described herein comprises a non-covalent binding unit.

In some embodiments, the non-covalent binding unit contributes to multimerization, such as dimerization, through non-covalent interactions, e.g. hydrophobic interactions. In some embodiments, the non-covalent binding unit has the ability to form multimers, such as dimers, via non-covalent interactions.

In some embodiments, the non-covalent binding unit contributes to multimerization through non-covalent interactions, e.g. hydrophobic interactions. In some embodiments, the non-covalent binding unit has the ability to form multimers via non-covalent interactions.

The non-covalent binding unit contributes to dimerization through non-covalent interactions, e.g. hydrophobic interactions. In some embodiments, the non-covalent binding unit has the ability to form dimers via non-covalent interactions.

In preferred embodiments, the amino acid sequence of the non-covalent binding unit is a non-immunogenic sequence.

In some embodiments, the amino acid sequence of the non-covalent binding unit is an artificial sequence.

In some embodiments, the amino acid sequence of the non-covalent binding unit is a naturally occurring sequence.

In some embodiments, the non-covalent binding unit is or comprises an immunoglobulin domain, such as an immunoglobulin constant domain (C domain), such as a carboxyterminal C domain (i.e. a CH3 domain), a CH1 domain or a CH2 domain, or a sequence that is substantially identical to the C domain or a variant thereof. In some embodiments, the non-covalent binding unit is a carboxyterminal C domain derived from IgG, such as derived from IgG3 or IgG1, preferably derived from IgG1.

It is preferred that if the non-covalent binding unit in one joint region comprises a CH3 domain, it does not in addition comprise a CH2 domain, and vice versa.

In some embodiments, the non-covalent binding unit comprises or consists of a carboxyterminal C domain derived from IgG3 with an amino acid sequence having at least 80% sequence identity to the amino acid sequence of SEQ ID NO: 3.

In preferred embodiments, the non-covalent binding unit comprises or consists of a carboxyterminal C domain derived from IgG3 with an amino acid sequence having at least 85% sequence identity to the amino acid sequence of SEQ ID NO: 3, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In preferred embodiments, the non-covalent binding unit comprises or consists of a carboxyterminal C domain derived from IgG3 with the amino acid sequence of SEQ ID NO: 3.

In some embodiments, the non-covalent binding unit comprises or consists of a carboxyterminal C domain derived from IgG3 with the amino acid sequence of SEQ ID NO: 3, wherein any one of the amino acids of the flexible unit has been substituted, deleted, or inserted for another amino acid, with the proviso that no more than 21 amino acids have been so substituted, deleted, or inserted, such as no more than 20 amino acids, such as no more than 19 amino acids, such as no more than 18 amino acids, such as no more than 17 amino acids, such as no more than 16 amino acids, such as no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids, such as no more than 6 amino acids, such as no more than 5 amino acids, such as no more than 4 amino acids, such as no more than 3 amino acids, such as no more than 2 amino acids, such as no more than 1 amino acid.

In some embodiments, the non-covalent binding unit comprises or consists of a CH3 domain derived from IgG1 with an amino acid sequence having at least 80% sequence identity to the amino acid sequence of SEQ ID NO: 4.

In some preferred embodiments, the non-covalent binding unit comprises or consists of a CH3 domain from IgG1 with an amino acid sequence having at least 85% sequence identity to the amino acid sequence according to SEQ ID NO: 4, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In some embodiments, the non-covalent binding unit comprises or consists of a CH3 domain derived from IgG1 with the amino acid sequence of SEQ ID NO: 3, wherein any one of the amino acids of the flexible unit has been substituted, deleted, or inserted for another amino acid, with the proviso that no more than 21 amino acids have been so substituted, deleted, or inserted, such as no more than 20 amino acids, such as no more than 19 amino acids, such as no more than 18 amino acids, such as no more than 17 amino acids, such as no more than 16 amino acids, such as no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids, such as no more than 6 amino acids, such as no more than 5 amino acids, such as no more than 4 amino acids, such as no more than 3 amino acids, such as no more than 2 amino acids, such as no more than 1 amino acid.

In some preferred embodiments, the non-covalent binding unit is or comprises CH3 of IgG1.

In other embodiments, the non-covalent binding unit is or comprises a leucine zipper motif.

A leucine zipper is a common three-dimensional structural motif in proteins where leucine side chains from one alpha helix interdigitate with those from another alpha helix, facilitating dimerization.

Leucine zippers are a dimerization motif of the bZIP (Basic-region leucine zipper) class of eukaryotic transcription factors. The bZIP domain is 60 to 80 amino acids in length with a highly conserved DNA binding basic region and a more diversified leucine zipper dimerization region. In some embodiments, the non-covalent binding unit is or comprises a leucine zipper motif derived from the bZIP class of eukaryotic transcription factors.

In some embodiments, the non-covalent binding unit is or comprises a Jun/Fos-based leucine zipper. In some embodiments, the non-covalent binding unit is or comprises a ATF6-based leucine zipper. In some embodiments, the non-covalent binding unit is or comprises a PAR-based leucine zipper. In some embodiments, the non-covalent binding unit is or comprises a C/EBPa-based leucine zipper. In some embodiments, the non-covalent binding unit is or comprises an OASIS-based leucine zipper.

In further preferred embodiments, the non-covalent binding unit is or comprises a leucine zipper motif (amino acids 308-336) from the CREB transcription factor (SEQ ID NO: 5).

In further preferred embodiments, the non-covalent binding unit comprises or consists of an amino acid sequence having at least 80% sequence identity to the amino acid sequence of SEQ ID NO: 5, such as at least 81% or at least 81%, 83%, 84%, 85%, 86%, 87%, 88%, 89%, 90%, 91%, 92%, 93%, 94%, 95%, 96%, 97%, 98% or at least 99% sequence identity.

In further preferred embodiments, the non-covalent binding unit comprises or consists of the amino acid sequence of SEQ ID NO: 5, wherein any one of the amino acids of the flexible unit has been substituted, deleted, or inserted for another amino acid, with the proviso that no more than 12, such as no more than 11, such as no more than 10, such as no more than 9, such as no more than 8, such as no more than 7, such as no more than 6, such as no more than 5, such as no more than 4 amino acids, such as no more than 3 amino acids, such as no more than 2 amino acids, such as no more than 1 amino acid.

In some embodiments, the joint region comprises a flexible unit and a binding unit which is either a covalent or non-covalent binding unit. In some embodiments, the joint region comprises a binding unit which comprises both, a covalent binding unit and a non-covalent binding unit.

In other embodiments, the joint region comprises a flexible unit, a covalent binding unit and a non-covalent binding unit. In some embodiments, the non-covalent binding unit is located between the antigenic unit and the covalent binding unit. In other embodiments, the covalent binding unit is located between the antigenic unit and the non-covalent binding unit.

In other embodiments, the joint region comprises a flexible unit and a non-covalent binding unit. In other embodiments, the joint region comprises a flexible unit and a covalent binding unit. In preferred embodiments, the flexible unit is located closest to the targeting unit, i.e. between the targeting unit and covalent- and/or non-covalent binding unit.

In some embodiments, the joint region further comprises a linker. In further embodiments, the linker is located between the covalent binding unit, and the non-covalent binding unit.

Non-Covalent Binding Unit that Facilitates Multimerization of/Joins More than Two Polypeptides

In addition to connecting the antigenic unit and the targeting unit, the non-covalent binding unit facilitates multimerization of/joins multiple polypeptides, such as two, three, four or more polypeptides, into a multimeric protein, such as a dimeric protein, a trimeric protein or a tetrameric protein.

In some embodiments, the non-covalent binding unit is or comprises a trimerization unit, such as a collagen-derived trimerization unit, such as a human collagen-derived trimerization domain, such as human collagen derived XVIII trimerization domain (see for instance A. Alvarez-Cienfuegos et al., Sci Rep 6, 28643 (2016)) or human collagen XV trimerization domain. Thus, in one embodiment, the non-covalent binding unit is a trimerization unit that comprises or consists of the nucleic acid sequence with SEQ ID NO: 158, or an amino acid sequence encoded by said nucleic acid sequence. In other embodiments, the trimerization unit is the C-terminal domain of T4 fibritin. Thus, in some embodiments, the non-covalent binding unit is a trimerization unit that comprises or consists of the amino acid sequence with SEQ ID NO: 159, or an nucleic acid sequence encoding said nucleic acid sequence.

In other embodiments, the non-covalent binding unit is or comprises a tetramerization unit, such as a domain derived from p53, optionally further comprising a hinge region as described below. Thus, in some embodiments, the non-covalent binding unit is a tetramerization unit that comprises or consists of the nucleic acid sequence with SEQ ID NO: 160, or an amino acid sequence encoded by said nucleic acid sequence, optionally further comprising a hinge region as described below.

### Specific Embodiments of the Joint Regions

In preferred embodiments, the joint region comprises hinge exon h1 and hinge exon h4 of IgG3. In further preferred embodiments, the joint region comprises or consists of an amino acid sequence having at least 40% sequence identity to the amino acid sequence of SEQ ID NO: 1, provided that the cysteine residues in the sequence are retained in their number and position, such as at least 50% sequence identity, at least 60%, at least 70%, at least 80% or at least 90% sequence identity.

In further preferred embodiments, the joint region comprises or consists of the amino acid sequence of SEQ ID NO: 1, provided that the cysteine residues in the sequence are retained in their number and position, wherein any one of the amino acids of the flexible unit has been substituted, deleted, or inserted for another amino acid, with the proviso that no more than 16 amino acids have been so substituted, deleted, or inserted, such as no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids, such as no more than 6 amino acids, such as no more than 5 amino acids, such as no more than 4 amino acids, such as no more than 3 amino acids, such as no more than 2 amino acids, such as no more than 1 amino acid.

In some embodiments, the joint region is hinge exon h1 and hinge exon h4 of IgG3. In other embodiments, the joint region consists of or comprises the amino acid sequence of SEQ ID NO: 1.

If the above-described joint region is the second joint region, said joint region comprises the hinge exons in the order h4 to h1, i.e. the above-described sequence is “flipped”, such that the flexible unit, h1, is closest to the second targeting unit.

In other preferred embodiments, the joint region comprises the middle and lower hinge regions of IgG1. In further preferred embodiments, the joint region comprises or consists of an amino acid sequence having at least 40% sequence identity to the amino acid sequence 5-23 of SEQ ID NO: 2, provided that the cysteine residues in the sequence are retained in their number and position, such as at least 50% sequence identity, at least 60%, at least 70%, at least 80% or at least 90% sequence identity.

In further preferred embodiments, the joint region comprises or consists of the amino acid sequence 5-23 of SEQ ID NO: 2, provided that the cysteine residues in the sequence are retained in their number and position, wherein any one of the amino acids of the flexible unit has been substituted, deleted, or inserted for another amino acid, with the proviso that no more than 11 amino acids have been so substituted, deleted, or inserted, such as no more than 10 amino acids, such as no more than 9 amino acids, such as no more than 8 amino acids, such as no more than 7 amino acids, such as no more than 6 amino acids, such as no more than 5 amino acids, such as no more than 4 amino acids, such as no more than 3 amino acids, such as no more than 2 amino acids, such as no more than 1 amino acid.

In some embodiments, the joint region is the middle and lower hinge region of IgG1. In other preferred embodiments, the joint region consists of or comprises the amino acid sequence 5-23 of SEQ ID NO: 2.

If the above-described joint region is the first joint region, said joint region comprises the hinge regions in the order lower hinge region to middle hinge region, i.e. the above-described sequence is “flipped”, such that the flexible unit, the lower hinge region, is closest to the first targeting unit.

In some embodiments, the joint region comprises hinge exon h1 and hinge exon h4 of IgG3 and/or the joint region comprising the middle and lower hinge region of IgG1 may further comprise a non-covalent biding region, e.g. the afore-described non-covalent binding regions, preferably an immunoglobulin constant domain.

### Targeting Unit

The tolerance-inducing construct of the disclosure comprises a first and a second targeting unit that targets antigen-presenting cells (APCs).

The first and the second targeting units are connected to the first and second joint regions as described herein, respectively.

The term “targeting unit” as used herein refers to a unit that delivers the construct of the disclosure to an antigen-presenting cell and interacts with surface molecules on the APC, e.g. binds to surface receptors on the APC, without inducing maturation of the cell. The APC internalizes the construct and presents the T cell epitopes comprised in the antigenic unit on MHC on its surface in an anti-inflammatory, tolerogenic manner, e.g. by not upregulating co-stimulatory signals and/or by upregulating inhibitory surface receptors and/or by promoting the secretion of inhibitory cytokines. In some embodiments, the targeting unit comprises or consists of a moiety that binds to a surface molecule on APC selected from the group consisting of TGFβ receptor (including TGFβR1, TGFβR2, and TGFβR3), IL10R, such as IL-10RA and IL10-RB, IL2R, IL4R, IL6R, IL11R and IL13R, IL27R, IL35R, IL37R, GM-CSFR, FLT3, CCR7, CD11b, CD11c, CD103, CD14, CD36, CD205, CD109, VISTA, MARCO, MHCII, CD83, SIGLEC, MGL/Clec10A, ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163, and CD141.

In a preferred embodiment, the targeting unit comprises or consists of a moiety that binds to a surface molecule on human (h) APCs selected from the group consisting of hTGFβ receptor (including hTGFβR1, hTGFβR2, and hTGFβR3), hIL-10R, such as hIL-10RA and hIL-10RB, hIL-2R, hIL-4R, hIL-6R, hIL-11R, hIL-13R, hIL-27R, hIL-35R, hIL-37R, hGM-CSFR, hFLT3, hCCR7, hCD11b, hCD11c, hCD103, hCD14, hCD36, hCD205, hCD109, hVISTA, hMARCO, hMHCII, hCD83, hSIGLEC, hClec10A (hMGL), hASGR (hASGR1/hASGR2), hCD80, hCD86, hClec9A, hClec12A, hClec12B, hDCIR2, hLangerin, hMR, hDC-Sign, hTreml4, hDectin-1, hPDL1, hPDL2, hHVEM, hCD163 and hCD141.

The moiety may be a natural ligand, an antibody or part thereof, e.g. a scFv, or a synthetic ligand.

In some embodiments, the moiety is an antibody or part thereof, e.g. a scFv, with specificity for any of the aforementioned receptors, whose binding to the receptor results in the T cell epitopes comprised in the antigenic unit being presented in an anti-inflammatory, tolerogenic manner.

In other embodiments, the moiety is a synthetic ligand with specificity for any of the aforementioned receptors, whose binding to the receptor results in the T cell epitopes comprised in the antigenic unit being presented in an anti-inflammatory, tolerogenic manner. Protein modelling may be used to design such synthetic ligands.

In other embodiments, the moiety is a natural ligand.

In some embodiments, natural ligand is selected from the group consisting of TGFβ, such as TGFβ1, TGFβ2 or TGFβ3, IL-10, IL2, IL4, IL6, IL11, IL13, IL27, IL35, IL37, GM-CSF, FLT3L, CCL19, CCL21, ICAM-1 (Intercellular Adhesion Molecule 1 also known as CD54), keratin, VSIG-3, SCGB3A2, CTLA-4, preferably the extracellular domain of CTLA-4, PD-1, preferably the extracellular domain of PD-1 and BTLA, preferably the extracellular domain of BTLA.

In other embodiments, the targeting unit is or comprises IL-10 or TGFβ, preferably human IL-10 or human TGFβ.

In other embodiments, the targeting unit comprises or consists of an amino acid sequence having at least 80% sequence identity to that of human TGFβ.

In other embodiments, the targeting unit comprises or consists of an amino acid sequence having at least 85% sequence identity to the amino acid sequence of human TGFβ, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human TGFβ, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence having at least 80% sequence identity to that of human IL-10 (SEQ ID NO: 66).

In other embodiments, the targeting unit comprises or consists of an amino acid sequence having at least 85% sequence identity to the amino acid sequence of human IL-10, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human IL-10, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human IL-10, or a nucleotide sequence encoding human IL-10.

In some embodiments, the targeting unit is or comprises SCGB3A2or VSIG-3, preferably human VSIG-3 or human SCGB3A2.

In other embodiments, the targeting unit comprises or consists of an amino acid sequence having at least 80% sequence identity to that of human SCGB3A2.

In other embodiments, the targeting unit comprises or consists of an amino acid sequence having at least 85% sequence identity to the amino acid sequence of human SCGB3A2, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human SCGB3A2, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human SCGB3A2, or a nucleotide sequence encoding human SCGB3A2.

In other embodiments, the targeting unit comprises or consists of an amino acid sequence having at least 80% sequence identity to that of human VSIG-3.

In other embodiments, the targeting unit comprises or consists of an amino acid sequence having at least 85% sequence identity to the amino acid sequence of human VSIG-3, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In another embodiments, the targeting unit comprises or consists of an amino acid sequence of human VSIG-3, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human VSIG-3, or a nucleotide sequence encoding human VSIG-3.

In other embodiments, the targeting unit is or comprises an antibody or part thereof, e.g. a scFv, with specificity for CD205.

### Antigenic Unit

The antigenic unit of the tolerance-inducing construct of the disclosure comprises one or more T cell epitopes of a self-antigen, including, but not limited to, a T reg epitope or inhibitory neoantigen, an allergen, an alloantigen or a xenoantigen.

The antigenic unit is located between the first and the second joint region as described herein.

In some embodiments, the antigenic unit comprises one or more T cell epitopes of a self-antigen, i.e. one T cell epitope of a self-antigen or more than one T cell epitope of a self-antigen, i.e. multiple T cell epitopes of a self-antigen. In one embodiment, the multiple T cell epitopes are of the same self-antigen, i.e. comprised in the same self-antigen. In another embodiment, the multiple T cell epitopes are of multiple different self-antigens, i.e. comprised in different self-antigens.

In some embodiments, where the antigenic unit comprises more than one T cell epitope, the antigenic unit comprises one or more linkers separating the T cell epitopes. In some embodiments, the antigenic unit comprises multiple antigens, e.g. multiple T cell epitopes of a self-antigen, an allergen, an alloantigen or a xenoantigen, wherein the T cell epitopes are preferably separated by a linkers. In yet other embodiments, the antigenic unit comprises multiple T cell epitopes of a self-antigen, an allergen, an alloantigen or a xenoantigen wherein each T cell epitope is separated from other antigens by linkers. An alternative way to describe the separation of each T cell epitope of a self-antigen, an allergen, an alloantigen or a xenoantigen from other T cell epitopes by linkers is that all but the terminal T cell epitopes, i.e. the antigen at the N-terminal start of the polypeptide or the C-terminal end of the polypeptide (i.e. located at the end of the antigenic unit that is not connected to the dimerization unit), are arranged in subunits, wherein each subunit comprises or consists of an antigen and a linker as described herein.

Hence, an antigenic unit comprising n antigens comprises n-1 subunits, wherein each subunit comprises a T cell epitope of a self-antigen, an allergen, an alloantigen or a xenoantigen, and a linker, and further comprises a terminal T cell epitope. In some embodiments, n is an integer of from 1 to 50, e.g. 3 to 50 or 15 to 40 or 10 to 30 or 10 to 25 or 10 to 20 or 15 to 30 or 15 to 25 or 15 to 20.

Linkers in the antigenic unit separate antigens comprised therein, e.g. epitopes. As described above, all T cell epitopes of a self-antigen, an allergen, an alloantigen or a xenoantigen, may be separated from each other by linkers and arranged in subunits.

In some embodiments, the linker is designed to be non-immunogenic. It may be a rigid linker, meaning that that it does not allow the two amino acid sequences that it connects to substantially move freely relative to each other. Alternatively, it may be a flexible linker, i.e. a linker that allows the two amino acid sequences that it connects to substantially move freely relative to each other.

Due to the separation of the antigens by the linkers, each T cell epitope of a self-antigen, an allergen, an alloantigen or a xenoantigen is presented in an optimal way to the immune system.

By way of example, myelin basic protein (MBP), proteolipid protein (PLP), myelin-associated glycoprotein (MAG), myelin oligodendrocyte glycoprotein (MOG) and myelin-associated basic oligodendrocytic protein (MOBP) have all been studied and proposed as self-antigens involved in multiple sclerosis (MS) and the antigenic unit may comprise e.g. one or more T cell epitopes of MBP, i.e. one T cell epitope of MBP or multiple T cell epitopes of MBP. Further, the antigenic unit may comprise multiple T cell epitopes of e.g. MOG and PLP, e.g. one or multiple T cell epitopes of MOG and one or multiple T cell epitopes of PLP.

In other embodiments, the antigenic unit comprises one or more T cell epitopes of an allergen, i.e. one T cell epitope of an allergen or more than one T cell epitope of an allergen, i.e. multiple T cell epitopes of an allergen. In one embodiment, the multiple T cell epitopes are of the same allergen, i.e. comprised in the same allergen. In other embodiments, the multiple T cell epitopes are of multiple different allergens, i.e. comprised in different allergens.

By way of example, Fel d1, Fel d4 and Fel d7 are three of the most prominent cat allergens, accounting for the majority of human cat allergies and the antigenic unit may comprise e.g. one or more T cell epitopes of Fel d1, i.e. one T cell epitope of Fel d1 or multiple T cell epitopes of Fel d1. Further, the antigenic unit may comprise multiple T cell epitopes of e.g. Fel d4 and Fel d7, e.g. one or multiple T cell epitopes of Fel d4 and one or multiple T cell epitopes of Fel d7.

In other embodiments, the antigenic unit comprises one or more T cell epitopes of an alloantigen/xenoantigen, i.e. one T cell epitope of an alloantigen/xenoantigen or more than one T cell epitope of an alloantigen/xenoantigen, i.e. multiple T cell epitopes of an alloantigen/xenoantigen. In some embodiments, the multiple T cell epitopes are of the same alloantigen/xenoantigen, i.e. comprised in the same alloantigen/xenoantigen. In other embodiments, the multiple T cell epitopes are of multiple different alloantigen/xenoantigens, i.e. comprised in different alloantigens/xenoantigens.

In some embodiments, the antigenic unit includes one T cell epitope. In other embodiments, the antigenic unit includes more than one T cell epitope, i.e. multiple T cell epitopes.

The tolerance-inducing construct of the disclosure may be an individualized treatment, i.e. designed for a particular subject/one patient. In other embodiments, the tolerance-inducing construct of the disclosure is for general use in a patient population or patients, i.e. an off-the-shelf treatment.

### Individualized Tolerance-Inducing Constructs

For individualized tolerance-inducing constructs, T cell epitopes are selected for inclusion into the antigenic unit, which T cell epitopes are optimized for the patient who will receive treatment with the construct. This will increase the therapeutic effect compared to an off-the-shelf treatment comprising the tolerance-inducing construct.

The antigenic unit of an individualized tolerance-inducing construct may be designed as follows, as exemplified for a patient suffering from MS:
1) The patient's HLA class I and/or HLA class II alleles are determined
2) T cell epitopes are identified comprised in one or more self-antigens (e.g. self-antigen which have been studied and proposed as self-antigens involved MS)
3) T cell epitopes are selected based on predicted binding to the patient's HLA class I and/or class II alleles
4) One or more tolerance-inducing test constructs are designed and produced, and the T cell epitopes are optionally arranged in the antigenic unit of the constructs as described in this application.

The T cell epitopes are selected in the method described above based on their predicted ability to bind to the patient's HLA class I/II alleles, i.e. selected in silico using predictive HLA-binding algorithms. After having identified relevant epitopes, the epitopes are ranked according to their ability to bind to the patient's HLA class I/II alleles and the epitopes that are predicted to bind best are selected to be included in the antigenic unit of the test constructs.

Any suitable HLA-binding algorithm may be used, such as one of the following: Available software analysis of peptide-MHC binding (IEDB, NetMHCpan and NetMHCIIpan) may be downloaded or used online from the following websites: www.iedb.org/services.healthtech.dtu.dk/service.php?NetMHCpan-4.0 services.healthtech.dtu.dk/service.php?NetMHCIIpan-3.2

### Off-the-Shelf Tolerance Inducing Constructs

The antigenic unit of an off-the-shelf tolerance inducing construct preferably includes hotspots of minimal T cell epitopes, i.e. one or more regions of an antigen that contain multiple minimal T cell epitopes (e.g. having a length of from 8-15 amino acids) that are predicted to be presented by different HLA alleles to cover a broad range of subjects, e.g. an ethnic population or even a world population.

By including such hotspots, chances are maximized that the construct will induce tolerance in a broad range of subjects.

### Further Description of the Antigenic Unit

The T cell epitope comprised in the antigenic unit of the construct of the disclosure has a length of from 7 to about 200 amino acids, with the longer T cell epitopes possibly including hotspots of minimal epitopes.

In some embodiments, the antigenic unit comprises T cell epitopes with a length of from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids, such as 25, 26, 27, 28, 29, 30, 31, 32, 33, 34, 35, 36, 37, 38, 39, 40, 41, 42, 43, 44, 45, 46, 47, 48, 49, or 50 amino acids.

T cell epitopes having a length of about 60 to 200 amino acids may be split into shorter sequences and included into the antigenic unit separated by the linkers which are described herein. By way of example, a T cell epitope having a length of 150 amino acids may be split into 3 sequences of 50 amino acids each, and included into the antigenic unit, with a linker separating the 3 sequences from each other.

In some embodiments, the length of one T cell epitope is such that the protein does not fold correctly. For example, Fel d 1, the most prominent cat allergen, is a protein formed by two heterodimers, with each dimer being composed of two chains, chain 1 comprising 70 amino acid residues and chain 2, comprising 90 or 92 residues. Including long T cell epitopes of both chains into the antigenic unit may result in the proteins folding correctly and, if more than one IgE on the subject's mast cells and basophiles binds the antigenic unit of the construct, might elicit and allergic reaction. If a longer T cell epitope is included in the antigenic unit, protein folding may be tested in vitro by e.g. ELISA, using an antibody against the protein (e.g. cat allergen) and determining whether the antibody binds to the T cell epitope.

In some embodiments, the T cell epitope has a length suitable for presentation by MHC (major histocompatibility complex). There are two primary classes of MHC molecules, MHC class I and MHC II. The terms MHC class I and MHC class II are interchangeably used herein with HLA class I and HLA class II. HLA (human leukocyte antigen) is a major histocompatibility complex in humans. Thus, in preferred embodiments, the antigenic unit comprises T cell epitopes having a length suitable for specific presentation on MHC class I or MHC class II. In some embodiments, the T cell epitope has a length of from 7 to 11 amino acids for MHC class I presentation. In other embodiments, the T cell epitope sequence has a length of from 9 to 60 amino acids, such as from 9 to 30 amino acids, such as 15 to 60 amino acids, such as 15 to 30 amino acids for MHC class II presentation. In preferred embodiments the T cell epitope has a length of 15 amino acids for MHC class II presentation.

The number of T cell epitopes in the antigenic unit may vary, and depends on the length and number of other elements included in the antigenic unit, e.g. T cell epitope linkers as described in this application.

In some embodiments, the antigenic unit comprises up to 3500 amino acids, such as from 60 to 3500 amino acids, e.g. from about 80 or about 100 or about 150 amino acids to about a 3000 amino acids, such as from about 200 to about 2500 amino acids, such as from about 300 to about 2000 amino acids or from about 400 to about 1500 amino acids or from about 500 to about 1000 amino acids.

In some embodiments, the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes. In other embodiments, the antigenic unit comprises 1 to 3 T cell epitopes, such as 1, 2, 3, or 1 to 5 T cell epitopes, such as 1, 2, 3, 4, 5, or 3 to 6 T cell epitopes, such as 3, 4, 5, 6, or 5 to 15 T cell epitopes, such as 5, 6, 7, 8, 9, 10, 11, 12, 13, 14, or 15 T cell epitopes, or 7 to 17 T cell epitopes, such as 7, 8, 9, 10, 11, 12, 13, 14, 15, 16, or 17 T cell epitopes, or 9 to 19 T cell epitopes, such as 9, 10, 11, 12, 13, 14, 15, 16, 17, 18, or 19 T cell epitopes.

In some embodiments, the T cell epitopes are randomly arranged in the antigenic unit. In other embodiments, on or more of the following methods for arranging them in the antigenic unit may be used.

In some embodiments, the T cell epitopes are arranged in the order of more antigenic to less antigenic in the direction from multimerization/dimerization unit to the end of the antigenic unit (see FIG. 1). Alternatively, particularly if the hydrophilicity/hydrophobicity varies greatly among the T cell epitopes, the most hydrophobic T cell epitope(s) may be positioned substantially in the middle of the antigenic unit and the most hydrophilic T cell epitope(s) is/are positioned closest to the multimerization/dimerization unit or the end of the antigenic unit.

In some embodiments, the T cell epitopes are arranged in the order of more antigenic to less antigenic in the direction from the multimerization unit to the end of the antigenic unit. Alternatively, particularly if the hydrophilicity/hydrophobicity varies greatly among the T cell epitopes, the most hydrophobic T cell epitope(s) may be positioned substantially in the middle of the antigenic unit and the most hydrophilic T cell epitope(s) is/are positioned closest to the multimerization unit or the end of the antigenic unit.

In some embodiments, the T cell epitopes are arranged in the order of more antigenic to less antigenic in the direction from dimerization unit to the end of the antigenic unit (see FIG. 1). Alternatively, particularly if the hydrophilicity/hydrophobicity varies greatly among the T cell epitopes, the most hydrophobic T cell epitope(s) may be positioned substantially in the middle of the antigenic unit and the most hydrophilic T cell epitope(s) is/are positioned closest to the dimerization unit or the end of the antigenic unit.

Since a true positioning in the middle of the antigenic unit is only possible if the antigenic unit comprises an odd number of T cell epitopes, the term “substantially” in this context refers to antigenic units comprising an even number of T cell epitopes, wherein the most hydrophobic T cell epitopes are positioned as close to the middle as possible.

By way of example, an antigenic unit comprises 5 T cell epitopes, which are arranged as follows: 1-2-3*-4-5; with 1, 2, 3*, 4 and 5 each being a different T cell epitope and—being a T cell epitope linker and * indicating the most hydrophobic T cell epitope, which is positioned in the middle of the antigenic unit.

In another example, an antigenic unit comprises 6 T cell epitopes, which are arranged as follows: 1-2-3*-4-5-6 or, alternatively, as follows: 1-2-4-3*-5-6; with 1, 2, 3*, 4, 5 and 6 each being a T cell epitope and—being a T cell epitope linker and * indicating the most hydrophobic T cell epitope, which is positioned substantially in the middle of the antigenic unit.

Alternatively, the T cell epitopes may be arranged alternating between a hydrophilic and a hydrophobic T cell epitope. Optionally, GC rich T cell epitopes are arranged in such a way, that GC clusters are avoided. In preferred embodiments, GC rich T cell epitopes are arranged such that there is at least one non-GC rich T cell epitope between them. In some embodiments, GC rich sequences encoding T cell epitopes are arranged such that there is at least one non-GC rich T cell sequence between them. GC rich sequences are sequences with a GC content of 60% or more, such as 65% or more, such as 70% or more, such as 75% or more, such as 80% or more.

If the antigenic unit comprises multiple T cell epitopes, the epitopes are preferably separated by T cell epitope linkers. This ensures that each T cell epitope is presented in an optimal way to the immune system. If the antigenic unit comprises n T cell epitopes, it preferably comprises n-1 T cell epitope linkers, separating each T cell epitope from one or two other T cell epitopes.

The T cell epitope linker is designed to be non-immunogenic and is preferably also a flexible linker, which allows for presenting the T cell epitope in an optimal manner to the immune system, even if the antigenic unit comprises a large number of T cell epitopes.

Preferably, the T cell epitope linker is a peptide consisting of from 4 to 20 amino acids, e.g. from 5 to 20 amino acids or 5 to 15 amino acids or 8 to 20 amino acids or 8 to 15 amino acids, such as 8, 9, 10, 11, 12, 13, 14, or 15 amino acids, 10 to 15 amino acids or 8 to 12 amino acids, such as 8, 9, 10, 11, or 12 amino acids. In particular embodiments, the T cell epitope linker consists of 10 amino acids.

All T cell epitope linkers comprised in the antigenic unit are preferably identical. If, however, one or more of the T cell epitopes comprises a sequence similar to that of the linker, it may be an advantage to substitute the neighboring T cell epitope linker with a linker of a different sequence. Also, if a T cell epitope/linker junction is predicted to constitute an epitope in itself, then it is preferred to use a T cell epitope linker of a different sequence.

Preferably, the T cell epitope linker is a serine(S) and/or glycine (G) rich linker, i.e. a linker comprising several serine and/or several glycine residues. Preferred examples are GGGGSGGGSS (SEQ ID NO: 75), GGGSG (SEQ ID NO: 76), GGGGS (SEQ ID NO: 77), SGSSGS (SEQ ID NO: 78), GGSGG (SEQ ID NO: 79), or multiple variants thereof such as GGGGSGGGGS (SEQ ID NO: 80), (GGGGS)m (SEQ ID NO:81), (GGGSS)m (SEQ ID NO: 82), (GGSGG)m (SEQ ID NO: 161), (GGGSG)m (SEQ ID NO: 83) or (SGSSGS)m (SEQ ID NO: 84), where m is an integer from 1 to 5, e.g., 1, 2, 3, 4, or 5, In preferred embodiments, m is 2. In other preferred embodiments, the serine and/or glycine rich linker further comprises at least one leucine (L) residue, such as at least 1 or at least 2 or at least 3 leucine residues, e.g. 1, 2, 3 or 4 leucine residues.

In some embodiments, the T cell epitope linker comprises or consists of LGGGS (SEQ ID NO: 85), GLGGS (SEQ ID NO: 86), GGLGS (SEQ ID NO: 87), GGGLS (SEQ ID NO: 88) or GGGGL (SEQ ID NO: 89). In other embodiments, the T cell epitope linker comprises or consists of LGGSG (SEQ ID NO: 90), GLGSG (SEQ ID NO: 91), GGLSG (SEQ ID NO: 92), GGGLG (SEQ ID NO: 93) or GGGSL (SEQ ID NO: 94). In yet other embodiments, the T cell epitope linker comprises or consists of LGGSS (SEQ ID NO: 95), GLGSS (SEQ ID NO: 96), or GGLSS (SEQ ID NO: 97).

In other embodiments, the T cell epitope linker comprises or consists of LGLGS (SEQ ID NO: 98), GLGLS (SEQ ID NO: 99), GLLGS (SEQ ID NO: 100), LGGLS (SEQ ID NO: 101), GLGGL (SEQ ID NO: 102) or (GLGGL)m (SEQ ID NO: 162). In other embodiments, the T cell epitope linker comprises or consists of LGLSG (SEQ ID NO: 103), GLLSG (SEQ ID NO: 104), GGLSL (SEQ ID NO: 105), GGLLG (SEQ ID NO: 106) or GLGSL (SEQ ID NO: 107). In other embodiments, the T cell epitope linker comprises or consists of LGLSS (SEQ ID NO: 108), or GGLLS (SEQ ID NO: 109).

In other embodiments, the T cell epitope linker is serine-glycine linker that has a length of 10 amino acids and comprises 1 or 2 leucine residues.

In some embodiments, the T cell epitope linker comprises or consists of LGGGSGGGGS (SEQ ID NO: 110), GLGGSGGGGS (SEQ ID NO: 111), GGLGSGGGGS (SEQ ID NO: 112), GGGLSGGGGS (SEQ ID NO: 113) or GGGGLGGGGS (SEQ ID NO: 114). In other embodiments, the T cell epitope linker comprises or consists of LGGSGGGGSG (SEQ ID NO: 115), GLGSGGGGSG (SEQ ID NO: 116), GGLSGGGGSG (SEQ ID NO: 117), GGGLGGGGSG (SEQ ID NO: 118) or GGGSLGGGSG (SEQ ID NO: 119). In other embodiments, the T cell epitope linker comprises or consists of LGGSSGGGSS (SEQ ID NO: 120), GLGSSGGGSS (SEQ ID NO: 121), GGLSSGGGSS (SEQ ID NO: 122), GGGLSGGGSS (SEQ ID NO: 123) or GGGSLGGGSS (SEQ ID NO: 124).

In further embodiments, the T cell epitope linker comprises or consists of LGGGSLGGGS (SEQ ID NO: 125), GLGGSGLGGS (SEQ ID NO: 126), GGLGSGGLGS (SEQ ID NO: 127), GGGLSGGGLS (SEQ ID NO: 128) or GGGGLGGGGL (SEQ ID NO: 129). In other embodiments, the T cell epitope linker comprises or consists of LGGSGLGGSG (SEQ ID NO: 130), GLGSGGLGSG (SEQ ID NO: 131), GGLSGGGLSG (SEQ ID NO: 132), GGGLGGGGLG (SEQ ID NO: 133) or GGGSLGGGSL (SEQ ID NO: 134). In other embodiments, the T cell epitope linker comprises or consists of LGGSSLGGSS (SEQ ID NO: 135), GLGSSGLGSS (SEQ ID NO: 136), GGLSSGGLSS (SEQ ID NO: 137).

In other embodiments, the T cell epitope linker comprises or consists of GSGGGA (SEQ ID NO: 138), GSGGGAGSGGGA (SEQ ID NO: 139), GSGGGAGSGGGAGSGGGA (SEQ ID NO: 140), GSGGGAGSGGGAGSGGGAGSGGGA (SEQ ID NO: 141) or GENLYFQSGG (SEQ ID NO: 142). In yet other embodiments, the T cell epitope linker comprises or consists of

(SEQ ID NO: 143)
SGGGSSGGGS,
(SEQ ID NO: 80)
GGGGSGGGGS,
(SEQ ID NO: 144)
SSGGGSSGGG,
(SEQ ID NO: 145)
GGSGGGGSGG,
(SEQ ID NO: 146)
GSGSGSGSGS,
(SEQ ID NO: 147)
GGGSSGGGSG,
(SEQ ID NO: 148)
GGGSSS,
(SEQ ID NO: 149)
GGGSSGGGSSGGGSS
or
(SEQ ID NO: 150)
GLGGLAAA.

In other embodiments, the T cell epitope linker is a rigid linker. Such rigid linkers may be useful to efficiently separate (larger) antigens and prevent their interferences with each other. In one embodiment, the subunit linker comprises or consists of

(SEQ ID NO: 163)
KPEPKPAPAPKP,
(SEQ ID NO: 164)
AEAAAKEAAAKA,
(SEQ ID NO: 165)
(EAAAK)mGS,
(SEQ ID NO: 39)
(EAAK)mGS,
(SEQ ID NO: 166)
PSRLEEELRRRLTEP
or
(SEQ ID NO: 167)
SACYCELS.

In other embodiments, the T cell epitope linker comprises or consists of the sequence TQKSLSLSPGKGLGGL (SEQ ID NO: 151). In other embodiments, the T cell epitope linker comprises or consists of the sequence SLSLSPGKGLGGL (SEQ ID NO: 168). In other embodiments, the T cell epitope linker comprises or consists of AAY or GPGPG (SEQ ID NO: 153).

In other embodiments, the T cell epitope linker is a GSAT linker, i.e. a linker comprising one or more glycine, serine, alanine and threonine residues, e.g. a linker comprising or consisting of the sequence GGSAGGSGSGSSGGSSGASGTGTAGGTGSGSGTGSG (SEQ ID NO: 154) or a SEG linker, i.e. a linker comprising one or more serine, glutamic acid and glycine residues, e.g. a linker comprising or consisting of the sequence

(SEQ ID NO: 155)
GGSGGGSEGGGSEGGGSEGGGSEGGGSEGGGSGGGS
or
(SEQ ID NO: 19)
ELKTPLGDTTHT.

In other embodiments, the T cell epitope linker is a cleavable linker, e.g. a linker which includes one or more recognition sites for endopeptidases, e.g. endopeptidases such as furin, caspases, cathepsins and the like. Cleavable linkers may be introduced to release free functional protein domains (e.g. encoded by larger antigens), which may overcome steric hindrance between such domains or other drawbacks due to interference of such domains, like decreased bioactivity, altered biodistribution.

Examples of T cell epitope linkers are disclosed in paragraphs [0098]-[0099] and in the recited sequences of WO 2020/176797A1 (in particular SEQ ID NOs: 37 to 65 and SEQ ID NOs: 67 to 76), which is incorporated herein by reference and in paragraphs to of US 2019/0022202A1, which is incorporated herein by reference.

### Allergens

The tolerance-inducing construct as described herein is useful for inducing tolerance to a range of different protein allergens, e.g. allergens that can be encoded by a nucleic acid sequence comprised in the polynucleotide of the constructs of the disclosure, including protein allergens that undergo post-translational modifications.

In some embodiments, the allergen is a food allergen. In some embodiments, the allergen is a shellfish allergen. In some embodiments, the allergen is tropomyosin, in other embodiments the allergen is Arginin kinase, myosin light chain, sarcoplasmic calcium binding protein, troponin C or Triose-phosphate isomerase or actin. In some embodiments, the allergen is Pan b 1. In some embodiments the antigen unit is Pan b 1 T cell epitope (251-270).

In some embodiments, the allergen is a cow's milk allergen. In some embodiments, the cow's milk allergen is Bos d 4, Bos d 5, Bos d 6, Bos d 7, Bos d 8, Bos d 9, Bos d 10, Bos d 11 or Bos d 12.

In some embodiments, the allergen is an egg allergen. In some embodiments, the egg allergen is ovomucoid, in other embodiments the egg allergen is ovalbumin, ovotransferin, conalbumin, Gal 3 3, egg lyaozyme or ovomucin.

One T cell epitope that is known in the art and has been studied in the context of egg allergy is OVA (257-264), with amino acid sequence SIINFEKL (SEQ ID NO: 168).

In some embodiments, the antigenic unit of the construct according to disclosure comprises the T cell epitope OVA (257-264). A pharmaceutical composition comprising said T cell epitope may be used in the treatment of egg allergy.

In some embodiments, the allergen is a fish allergen. In some embodiments, the fish allergen is a parvalbumin. In other embodiments the fish allergen is enolase, aldolase or vitellogenin. In some embodiments, the allergen is a fruit allergen. In some embodiments, the fruit allergen is pathgenesis related protein 10, profilin, nsLTP, thaumatin-like protein, gibberellin regulated protein, isoflavone reductase related protein, class 1 chitinase, beta 1,3 glucanase, germin like protein, alkaline serine protease, pathogenesis-related protein 1, actinidin, phytocyctatin, kiwellin, major latex protein, cupin or 2S albumin. In some embodiments, the allergen is a vegetable allergen. In some embodiments, the vegetable allergen is pathgenesis related protein 10, profilin, nsLTP type 1, nsLTP type protein 2, osmotin-like protein, isoflavone reductase-like protein, beta-fructofuranosidase, PR protein TSI-1, cyclophilin or FAD containing oxidase.

In some embodiments, the allergen is a wheat allergen. In some embodiments, the wheat allergen is Tri a 12, Tri a 14, Tri a 15, Tri a 18, Tri a 19, Tri a 20, Tri a 21, Tri a 25, Tri a 26, Tri a 27, Tri a 28, Tri a 29, Tri a 30, Tri a 31, Tri a 32, Tri a 33, Tri a 34, Tri a 35, Tri a 36, Tri a 37 or Tri a 38. In some embodiments, the allergen is a soy allergen. In some embodiments, the soy allergen is Gly m 1, Gly m 2, Gly m 3, Gly m 4, Gly m 5, Gly m 6, Gly m 7 or Gly m 8. In other embodiments the soy allergen is Gly m agglutinin, Gly m Bd28K, Gly m 30 kD, Gly m CPI or Gly m TI. In some embodiments, the allergen is a peanut allergen. In some embodiments, the peanut allergen is Ara h 1, Ara h 2, Ara h 3, Ara h 5, Ara h 6, Ara h 7, Ara h 8, Ara h 9, Ara h 10, Ara h 11, Ara h 12, Ara h 13, Ara h 14, Ara h 15, Ara h 16, or Ara h 17. In some embodiments, the allergen is a tree nut or seed allergen. In some embodiments, the allergen is 11S globulin, 7S globulin, 2S globulin, PR10, PR-14 nsLTP, Oleosin or profilin.

In other embodiments the food allergen is buckwheat, celery, a color additive, garlic, gluten, oats, legumes, maize, mustard, poultry, meat, rice, sesame.

In some embodiments, the allergen is a bee venom allergen. In some embodiments, the bee venom allergen is Phospholipase A2, Hyaluronidase, acid phosphatase, melittin, allergen C/DPP, CRP/Icarapin or vitellogenin. In some embodiments, the allergen is a vespid allergen. In some embodiments, the vespid allergen is Phospholipase A1, hyaluronidase, protease, antigen 5, DPP IV or vitellogenin.

In some embodiments, the allergen is a latex allergen. In some embodiments, the latex allergen is Hev b 1, Hev b 2, Hev b 3, Hev b 4, Hev b 5, Hev b 6, Hev b 7, Hev b 8, Hev b 9, Hev b 10, Hev b 11, Hev b 12, Hev b 13, Hev b 14, or Hev b 15.

In some embodiments, the allergen is a dust mite allergen. In some embodiments the allergen is a house dust mite allergen. In some embodiments, the allergen is a storage dust allergen. In some embodiments, the house dust mite allergen is Der p 1, Der p 2, Der p 3, Der p 4, Der p 5, Der p 7, Der p 8, Der p 10, Der p 11, Der p 21, or Der p 23. In some embodiments the antigen unit is the Der p 1 T cell epitope (111-139). In some embodiments, the house dust mite allergen is Der f 1, Der f 2, Der f 3, Der f 7, Der f 8 or Der f 10. In some embodiments, the house dust mite allergen is Blot t 1, Blot t 2, Blot t 3, Blot t 4, Blot t 5, Blot t 8, Blot t 10, Blot t 12 or Blot t 21.

In some embodiments, the allergen is a cockroach allergen. In some embodiments, the cockroach allergen is Bla g 1, Bla g 2, Bla g 3, Bla g 4, Bla g 5, Bla g 6, Bla g 7, Bla g 8 or Bla g 11. In some embodiments, the cockroach allergen is Per a 1, Per a 2, Per a 3, Per a 6, Per a 7, Per a 9 or Per a 10.

In some embodiments, the allergen is a mould allergen. In some embodiments, the mould allergen is an Aspergillus fumigatus allergen. In some embodiments, the Aspergillus fumigatus allergen is Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 14, Asp f 15, Asp f 16, Asp f 17, Asp f 18, Asp f 22, Asp f 23, Asp f 27, Asp f 28, Asp f 29 or Asp f 34.

In some embodiments, the allergen is a fungal allergen. In some embodiments, the fungal allergen is a Malassezia allergen. In some embodiments, the Malassezia allergen is Mala f 1, Mala f 2, Mala f 3, Mala f 4, Mala f 5, Mala f 6, Mala f 7, Mala f 8, Mala f 9, Mala f 10, Mala f 11, Mala f 12 or Mala f 13 or MGL_1204.

In some embodiments, the allergen is furry animal allergen. In some embodiments, the allergen is a dog allergen. In some embodiments, the dog allergen is Can f 1, Can f 2, Can f 3, Can f 4, Can f 5, or Can f 6. In some embodiments, the allergen is a horse allergen. In some embodiments, the horse allergen is Ecu c 1, Ecu c 2, Ecu c 3 or Ecu c 4. In some embodiments, the allergen is a cat allergen. In some embodiments, the cat allergen is Fel d 1, Fel d 2, Fel d 3, Fel d 4, Fel d 5, Fel d 6, Fel d 7, or Fel d 8. In some embodiments, the allergen is a laboratory animal allergen. In some embodiments, the allergen is Lipocalin, urinary prealbumin, secretoglobulin or serum albumin.

In some embodiments, the allergen is a pollen allergen. In some embodiments, the allergen is a grass pollen allergen. In some embodiments, the grass pollen allergen is a timothy grass, orchard grass, Kentucky bluegrass, perennial rye, sweet vernal grass, bahia grass, johnson grass or Bermuda grass allergen. In some embodiments the grass pollen allergen is Phi p 1, Phl p 2, Phl p 3, Phl p 4, Phl p 5, Phl p 6, Phl p 7, Phi p 11, Phi p 12 or Phl p 13.

In some embodiments, the allergen is a tree pollen allergen. In some embodiments, the tree pollen allergen is an alder, birch, hornbeam, hazel, European hophornbeam, chestnut, European beech, white oak, ash, privet, olive, lilac, cypress or cedar pollen allergen. In some embodiments, the tree pollen allergen is Aln g 1 or Aln g 4, Bet v 1, Bet v 2, Bet v 3, Bet v 4, Bet v 6 or Bet v 7, Car b 1, Cor a 1, Cor a 2, Cor a 6, Cor a 8, Cor a 9, Cor a 10, Cor a 11, Cor a 12, Cor a 13, Cor a 14, Ost c 1, Cas 1, Cas 15, Cas 18, or Cas 19, Fag s 1, Que a 1, Fra e 1, Lig v 1, Ole e 1, Ole e 2, Ole e 3, Ole e 4, Ole e 5, Ole e 6, Ole e 7, Ole e 8, Ole e 9, Ole e 10, Ole e 11, or Ole e 12, Syr v 1, Cha o 1, Cha o 2, Cry j 1, Cry j 2, Cup s 1, Cup s 3, Jun a 1, Jun a 2, Jun a 3, Jun o 4, Jun v 1, Jun v 3, Pla a 1, Pla a 2 or Pla a 3 or Pla or 1, Pla or 2 or Pla or 3. In some embodiments, the antigen unit is the Bet v 1 T cell epitope (139-152).

In some embodiments, the allergen is a weed pollen allergen. In some embodiments the weed allergen is a ragweed, mugwort, sunflower, feverfew, pellitory, English plantain, annual mercury, goosefoot, Russian thistle or amaranth pollen allergen. In some embodiments the ragweed pollen allergen is Amb a 1, Amb a 4, Amb a 6, Amb a 8, Amb a 9, Amb a 10, or Amb a 11. In some embodiments the mugwort pollen allergen is Art v 1, Art v 3, Art v 4, Art v 5, or Art v 6. In some embodiments, the sunflower pollen allergen is Hel a 1 or Hel a 2. In some embodiments, the pellitory pollen allergen is Par j 1, Par j 2, Par j 3 or Par j 4. In some embodiments, the English plantain pollen allergen is Pla l 1. In some embodiments, the annual mercury pollen allergen is Mer a 1. In some embodiments, the goosefoot pollen allergen is Che a 1, Che a 2 or Che a 3. In some embodiments, the Russian thistle pollen allergen is Sal k 1, Sal k 4 or Sal k 5. In some embodiments, the Amaranth pollen allergen is Ama r 2.

In yet other embodiments the allergen is selected form environmental allergens such as insects, cockroaches, house dust mites or mold.

In some embodiments, the allergic disease is allergic rhinitis, asthma, atopic dermatitis, allergic gastroenteropathy, contact dermatitis, drug allergy or combinations thereof.

Allergy to drugs affect more than 7% of the general population. The constructs of the disclosure induce tolerance towards immunogenic epitopes present in such a drug and thus will allow affected patients to continue treatment with the drug and receive the benefits from the drug treatment.

Thus, in some embodiments, the allergen is comprised in a drug with unwanted immunogenicity. In some embodiments, the allergen is Factor VIII. In some embodiments, the allergen is insulin. In some embodiments, the allergen is one or more monoclonal antibodies used for therapy.

### Self-Antigens

In other embodiments, the present tolerance-inducing construct contains T cell epitopes comprised in a self-allergen that is involved in an autoimmune disease. This allows for the antigen-specific down-regulation of the part of the immune system responsible for the autoimmune disease without inhibiting the immune system in general.

In some embodiments, the autoimmune disease is multiple sclerosis (MS). In some embodiments, the self-antigen is myelin oligodendrocyte glycoprotein (MOG). In other embodiments the self-antigen is MAG, MOBP, CNPase, S100beta or transaldolase. In some embodiments, the self-antigen is myelin basic protein (MBP). In some embodiments, the self-antigen is myelin proteolipid protein (PLP).

In the examples we provide constructs for multiple sclerosis including either a short (35-55 amino acids) or a longer (27-63 amino acids) T cell epitope from myelin oligodendrocyte glycoprotein (MOG). MOG is a member of the immunoglobulin superfamily and is expressed exclusively in the central nervous system. MOG (35-55) is able to induce autoantibody production and relapsing-remitting neurological disease, causing extensive plaque-like demyelination. Autoantibody response to MOG (35-55) has been observed in MS patients and MOG (35-55)-induced experimental autoimmune encephalomyelitis (EAE) in C57/BL6 mice and Lewis rats.

Other MS-relevant T cell epitopes that are known in the art and have been studied include the following:

| T cell epitope | Sequence |
|---|---|
| PLP (139-151)* | HCLGKWLGHPDKF (SEQ ID NO: 169) |
| PLP (131-159) | AHSLERVCHCLGKWLGHPDKFVGITYALT (SEQ ID NO: 170) |
| PLP (178-191)* | NTWTTCQSIAFPSK (SEQ ID NO: 58) |
| PLP (170-199) | AVPVYIYFNTWTTCQSIAFPSKTSASIGSL (SEQ ID NO: 57) |
| MBP (84-104)* | VHFFKNIVTPRTPPPSQGKGR (SEQ ID NO: 56) |
| MBP (76-112) | RTQDENPVVHFFKNIVTPRTPPPSQGKGRGLSLSRF (SEQ ID |
| | NO: 42) |

*T cell epitope-induced EAE observed

In preferred embodiments, the antigenic unit of the construct of the disclosure includes one or more T cell epitopes selected from the group consisting of MOG (35-55), MOG (27-63), PLP (139-151), PLP (131-159), PLP (178-191), PLP (170-199), MBP (84-104) and MBP (76-112). A pharmaceutical composition comprising such a construct may be used in the treatment of MS.

In some embodiments, the autoimmune disease is type 1 diabetes mellitus. In some embodiments, the self-antigen is glutamic acid decarboxylase 65-kilodalton isoform (GAD65), which is a self-antigen involved in type 1 diabetes mellitus. In some other embodiments, the self-antigen is insulin, IA-2 or ZnT8. In yet some other embodiments, the self-antigen is IGRP, ChgA, IAPP, peripherin, tetraspanin-7, GRP78, Urocortin-3 or Insulin gene enhancer protein isl-1.

In some embodiments, the autoimmune disease is celiac disease. In some embodiments, the self-antigen is α-gliadin, γ-gliadin, w-gliadin, low molecular weight glutenin, high molecular weight glutenin, hordein, secalin or avenin b. In some embodiments, the antigenic unit comprises the T cell epitope α-gliadin (76-95).

In some embodiments, the autoimmune disease is rheumatoid arthritis. In some embodiments, the self-antigen is collagen. In some embodiments, the self-antigen is heat shock protein 60 (HSP60). In some embodiments, the self-antigen is Band 3. In some embodiments, the self-antigen is small nuclear ribonucleoprotein D1 (SmD1). In some embodiments, the self-antigen is the acetylcholine receptor (AChR). In some embodiments, the self-antigen is myelin protein zero (PO).

In some embodiments, the autoimmune disease is chronic inflammatory demyelinating polyradiculoneuropathy (CIDP) and the self-antigen is neurofascin 155. In other embodiments, the autoimmune disease is Hashimoto's thyroiditis (HT) and the self-antigen is thyroid peroxidase and/or thyroglobulin. In other embodiments, the autoimmune disease is pemphigus foliaceus and the self-antigen is desmosome-associated glycoprotein. In other embodiments, the autoimmune disease is pemphigus vulgaris and the self-antigen is desmoglein 3. In other embodiments, the autoimmune disease is thyroid eye disease (TED) and the self-antigen is calcium binding protein (calsequestrin). In other embodiments, the autoimmune disease is Grave's disease and the self-antigen is thyroid stimulating hormone receptor. In other embodiments, the autoimmune disease is primary biliary cirrhosis (PBC) and the self-antigen is antimitochondrial antibodies (AMAs), antinuclear antibodies (ANA), Rim-like/membrane (RL/M) and/or multiple nuclear dot (MND). In other embodiments, the autoimmune disease is myasthenia gravis and the self-antigen is acetylcholine receptor. In other embodiments, the autoimmune disease is insulin-resistant diabetes and the self-antigen is insulin receptor. In other embodiments, the autoimmune disease is autoimmune hemolytic anemia and the self-antigen is erythrocytes. In other embodiments, the autoimmune disease is rheumatoid arthritis and the self-antigens are citrullinated, homocitrullinated proteins and the Fc portion of IgG. In other embodiments, the autoimmune disease is psoriasis and the self-antigens are cathelicidin (LL-37), disintegrin-like and metalloprotease domain containing thrombospondin type 1 motif-like 5 (ADAMTSL5), phospholipase A2 group IVD (PLA2G4D), heterogeneous nuclear ribonucleoprotein A1 (hnRNP-A1) and keratin 17.

### Signal Peptide

In some embodiments, the construct of the disclosure is a polynucleotide which further comprises a nucleotide sequence encoding a signal peptide. The signal peptide is either located at the N-terminal end of the targeting unit or the C-terminal end of the targeting unit, depending on the orientation of the targeting unit in the polypeptide (FIG. 1). The signal peptide is designed to allow secretion of the polypeptide encoded by the nucleic acid comprised in the polynucleotide in the cells transfected with said polynucleotide.

Any suitable signal peptide may be used. Examples of suitable peptides are a human Ig VH signal peptide or the signal peptides which are naturally present at the N-terminus of any of the targeting units described herein, e.g. a human signal peptide of human IL-10 or a human signal peptide of human TGFβ.

Thus, in some embodiments, the polynucleotide comprises a nucleotide sequence encoding a human IL-10 signal peptide and preferably comprises a nucleotide sequence encoding a human IL-10 targeting unit. In other embodiments, the polynucleotide comprises a nucleotide sequence encoding a human Ig VH signal peptide and preferably comprises a nucleotide sequence encoding a scFv, e.g. human anti-DEC205.

In some embodiments, the polynucleotide comprises a nucleotide sequence encoding a signal peptide that comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%, sequence identity to the amino acid sequence of SEQ ID NO: 6.

In preferred embodiments, the polynucleotide comprises a nucleotide sequence encoding a signal peptide that comprises the amino acid sequence of SEQ ID NO: 6. In other embodiments, the polynucleotide comprises a nucleotide sequence encoding a signal peptide that consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% to the amino acid sequence of SEQ ID NO: 6.

In other preferred embodiments, the polynucleotide which comprises a nucleotide sequence encoding a signal peptide with the amino acid sequence of SEQ ID NO: 6.

In some embodiments, the signal peptide comprises or consists of the amino acid sequence of SEQ ID NO: 6, wherein any one of the amino acids of the signal peptide has been substituted, deleted, or inserted for another amino acid, with the proviso that no more than 5 amino acids have been so substituted, deleted, or inserted, such as no more than 4 amino acids, such as no more than 3 amino acids, such as no more than 2 amino acids or no more than 1 amino acid.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used.

Amino acid sequence variants may be prepared by introducing appropriate changes into the nucleotide sequence encoding the tolerance-inducing construct, or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person in the art. Any combination of deletion, insertion, and substitution can be made to arrive at the final construct, provided that the final construct possesses the desired characteristics. For example, deletions, insertions or substitutions of amino acid residues may produce a silent change and result in a functionally equivalent peptide/polypeptide.

Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e. like-for-like substitution such as basic for basic, acidic for acidic, polar for polar etc. and non-conservative substitutions, i.e. from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, aaline, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-l-phenylalanine*, L-allyl-glycine*, β-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino) #, L-Tyr (methyl)*, L-Phe (4-isopropyl)*, L-Tic (1,2,3,4-tetrahydroisoquinoline-3-carboxyl acid)*, L-diaminopropionic acid * and L-Phe (4-benzyl)*.

In the paragraph above, * indicates the hydrophobic nature of the substituting residue, whereas # indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation involves the presence of one or more amino acid residues in peptoid form.

### Polynucleotides

The tolerance-inducing construct of the disclosure may be in the form of a polynucleotide.

A further aspect of the disclosure is a polynucleotide comprising a nucleotide sequence encoding a polypeptide, the polypeptide comprising, in the specified order:
a. a first targeting unit,
b. a first joint region;
c. an antigenic unit;
d. a second joint region; and
e. a second targeting unit; wherein the antigenic unit comprises one or more T cell epitopes of a self-antigen, an allergen, an alloantigen or a xenoantigen.

The polynucleotide may be a DNA or RNA, including genomic DNA, cDNA and mRNA, either double stranded or single stranded. In preferred embodiments, the construct is a DNA plasmid, i.e. the polynucleotide is a DNA.

It is preferred that the polynucleotide is optimized for use in the species to which it is administered. For administration to a human, it is thus preferred that the polynucleotide sequence is human codon optimized.

### Polypeptides and Multimeric/Dimeric Proteins

The tolerance-inducing construct of the disclosure may be in the form of a polypeptide encoded by the polynucleotide as described above.

A further aspect of the disclosure is a polypeptide, comprising in the specified order:
a. a first targeting unit, a first joint region;
b. an antigenic unit;
c. a second joint region; and
d. a second targeting unit; wherein the antigenic unit comprises one or more T cell epitopes of a self-antigen, an allergen, an alloantigen or a xenoantigen.

The polypeptide may be expressed in vitro for production of the tolerance-inducing construct, e.g. for production of a pharmaceutical composition comprising the construct, or the polypeptide may be expressed in vivo as a result of the administration of the polynucleotide to a subject, as described above. Due to the presence of the multimerization/dimerization unit, multimeric/dimeric proteins are formed when the polypeptide is expressed, i.e. by joining multiple polypeptides via their respective multimerization/dimerization units.

### Multimeric Proteins

A further aspect of the disclosure is a multimeric protein consisting of multiple polypeptides, wherein each of the polypeptides comprises, in the specified order,
a. a first targeting unit, a first joint region;
b. an antigenic unit;
c. a second joint region; and
d. a second targeting unit; wherein the antigenic unit comprises one or more T cell epitopes of a self-antigen, an allergen, an alloantigen or a xenoantigen, and wherein the multiple polypeptides are linked to each other via their respective first joint regions and via their respective second joint region.

The multimeric protein may be prepared by expression of the polypeptide in vitro.

Thus, a further aspect of the disclosure is a method for preparing a multimeric protein consisting of multiple polypeptides, wherein each of the polypeptides comprises, in the specified order,
a. a first targeting unit, a first joint region;
b. an antigenic unit;
c. a second joint region; and
d. a second targeting unit; wherein the antigenic unit comprises one or more T cell epitopes of a self-antigen, an allergen, an alloantigen or a xenoantigen,
   and wherein the multiple polypeptides are linked to each other via their respective first joint regions and via their respective second joint region, wherein the method comprises:
a. transfecting cells with a polynucleotide comprising a nucleotide sequence encoding the polypeptide;
b. culturing the cells;
c. collecting the multimeric protein from the cells; and
d. isolating and purifying the fraction of multimeric proteins, wherein the multiple polypeptides are linked to each other via their respective first joint regions and via their respective second joint regions.

Isolation of the multimeric protein in step d. and the optional purification can be carried out by methods known in the art, including precipitation, differential solubilization and chromatography.

The multimeric protein of the disclosure may be used as the active ingredient in a protein vaccine for the prophylactic or therapeutic treatment of autoimmune diseases, allergic disease and graft rejection.

The multimeric/dimeric proteins may be homomultimers or hetereomultimers, e.g. if the protein is a dimeric protein, the dimeric protein may be a homodimer, i.e. a dimeric protein wherein the two polypeptide chains are identical and consequently comprise identical units and thus antigen sequences, or the dimeric protein may be a heterodimer comprising two polypeptide chains, wherein polypeptide chain 1 comprises different antigen sequences in its antigenic unit than polypeptide 2. The latter may be relevant if the number of antigens for inclusion into the antigenic unit would exceed an upper size limit for the antigenic unit. It is preferred that the dimeric protein is a homodimeric protein.

### Dimeric Proteins

A further aspect of the disclosure is a dimeric protein consisting of two polypeptides, wherein each of the polypeptides comprises, in the specified order,
a. a first targeting unit, a first joint region;
b. an antigenic unit;
c. a second joint region; and
d. a second targeting unit; wherein the antigenic unit comprises one or more T cell epitopes of a self-antigen, an allergen, an alloantigen or a xenoantigen,
   and wherein the two polypeptides are linked to each other via their respective first joint regions and via their respective second joint region.

The dimeric protein may be prepared by expression of the polypeptide in vitro.

Thus, a further aspect of the disclosure is a method for preparing a dimeric protein consisting of two polypeptides, wherein each of the polypeptides comprises, in the specified order,
a. a first targeting unit, a first joint region;
b. an antigenic unit;
c. a second joint region; and
d. a second targeting unit; wherein the antigenic unit comprises one or more T cell epitopes of a self-antigen, an allergen, an alloantigen or a xenoantigen,
   and wherein the two polypeptides are linked to each other via their respective first joint regions and via their respective second joint region, wherein the method comprises:
a. transfecting cells with a polynucleotide comprising a nucleotide sequence encoding the polypeptide;
b. culturing the cells;
c. collecting the dimeric protein from the cells; and
d. isolating and purifying the fraction of dimeric proteins, wherein the two polypeptides are linked to each other via their respective first joint regions and via their respective second joint regions.

Isolation of the dimeric protein in step d) and the purification can be carried out by methods known in the art, including precipitation, differential solubilization and chromatography.

The dimeric protein of the disclosure may be used as the active ingredient in a protein vaccine for the prophylactic or therapeutic treatment of autoimmune diseases, allergic disease and graft rejection.

### Vectors

The polynucleotide sequence of the tolerance-inducing construct may be a DNA polynucleotide comprised in a vector suitable for transfecting a host cell and expression of a polypeptide or multimeric/dimeric protein encoded by the polynucleotide, i.e. an expression vector, such as a DNA plasmid or viral vector, preferably a DNA plasmid. In another embodiment, the vector is suitable for transfecting a host cell and expression of an mRNA encoding for the polypeptide or multimeric/dimeric protein.

The vectors of the invention may be any molecules which are suitable to carry foreign nucleic acid sequences, such as DNA or RNA, into a cell, where they can be expressed, i.e. expression vectors.

In one embodiment, the vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In another embodiment, the vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

In a preferred embodiment, the vector is a DNA vector, more preferably a DNA plasmid.

Preferably, the vector allows for easy exchange of the various units described above, particularly the antigenic unit in case of individualized tolerance-inducing constructs.

Thus, the disclosure provides a vector comprising a polynucleotide comprising a nucleotide sequence encoding a polypeptide, the polypeptide comprising, in the specified order:
a. a first targeting unit, a first joint region;
b. an antigenic unit comprising at least one T cell epitope;
c. a second joint region; and
d. a second targeting unit, wherein the antigenic unit comprises one or more T cell epitopes of a self-antigen, an allergen, an alloantigen or a xenoantigen.

In some embodiments, the vector may be pALD-CV77 or any other vector which does not comprise bacterial nucleotide sequences which are known to trigger an immune response in an unfavourable way, when introduced into a subject. The antigenic unit may be exchanged with an antigenic unit cassette restricted by a convenient restriction enzyme, e.g. the SfiI restriction enzyme cassette where the 5′ site is incorporated in the nucleotide sequence encoding the GLGGL (SEQ ID NO: 102) and/or GLSGL (SEQ ID NO: 40) unit linker and the 3′ site is included after the stop codon in the vector.

In preferred embodiments, the vector is a DNA plasmid and the polynucleotide is DNA.

### Dna Plasmids

A plasmid is a small, extrachromosomal DNA molecule within a cell that is physically separated from chromosomal DNA and can replicate independently. Plasmids are mostly found as small circular, double-stranded DNA molecules in bacteria; however, plasmids are sometimes present in archaea and eukaryotic organisms. Artificial plasmids are widely used as vectors in molecular cloning, serving to deliver and ensure high expression of recombinant DNA sequences within host organisms. Plasmids comprise several important features, including a feature for selection of cells comprising the plasmid, such as for example a gene for antibiotic resistance, an origin of replication, a multiple cloning site (MCS) and promoters for driving the expression of the inserted gene(s) of interest.

Generally, promoters are sequences capable of attracting initiation factors and polymerases to the promoter, so that a gene is transcribed. Promoters are located near the transcription start sites of genes, upstream on the DNA. Promoters can be about 100-1000 base pairs long. The nature of the promoter is usually dependent on the gene and product of transcription and type or class of RNA polymerase recruited to the site. When the RNA polymerase reads the DNA of the plasmid, an RNA molecule is transcribed. After processing, the mRNA will be able to be translated numerous times, and thus result in many copies of the proteins encoded by the genes of interest, when the ribosome translates the mRNA into protein. Generally, the ribosome facilitates decoding by inducing the binding of complementary tRNA anticodon sequences to mRNA codons. The tRNAs carry specific amino acids that are chained together into a polypeptide as the mRNA passes through and is “read” by the ribosome. Translation proceeds in three phases, initiation, elongation, and termination. Following the translation process, the polypeptide folds into an active protein and performs its functions in the cell or is exported from the cell and performs its functions elsewhere, sometimes after a considerable number of posttranslational modifications.

When a protein is destined for export out of the cell, a signal peptide directs the protein into the endoplasmic reticulum, where the signal peptide is cleaved off and the protein is transferred to the cell periphery after translation has terminated.

The DNA plasmid of the present invention is not limited to any specific plasmid, the skilled person will understand that any plasmid with a suitable backbone can be selected and engineered by methods known in the art to comprise the elements and units of the present disclosure.

### Host Cell

In some embodiments, the present disclosure provides a host cell comprising a vector as described herein.

In some embodiments, the present disclosure provides a host cell comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a vector comprising the polynucleotide.

Suitable host cells include prokaryotes, yeast, insect or higher eukaryotic cells. In preferred embodiments, the host cell is a human cell, preferably the cell of a human individual suffering from an immune disease and being in need of prophylactic or therapeutic treatment with the construct of the disclosure.

### Polycistronic Vectors

In some embodiments, the above-described vector is a polycistronic vector that allows the expression of the polypeptide of the disclosure and, in addition, the expression of one or more immunoinhibitory compounds as separate molecules.

A further aspect of the disclosure is a vector comprising:
(A) a polynucleotide comprising a nucleotide sequence encoding a polypeptide comprising, in the specified order
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; and
(B) one or more nucleic acid sequences encoding one or more immunoinhibitory compounds,
wherein the vector allows for the co-expression of the polypeptide and the one or more immunoinhibitory compounds as separate molecules.

The one or more immunoinhibitory compounds help to generate or promote an environment that favors the presentation of the epitopes in the antigenic unit in a tolerance inducing manner, or by e.g. favoring the induction of tolerance maintaining cells or helping to maintain such cells.

The polycistronic vector of the disclosure may be any suitable vector, e.g. a DNA plasmid or viral vector, such as a retroviral vector. In preferred embodiments, the vector is a polycistronic DNA plasmid. The polycistronic vector of the disclosure will be illustrated discussing a DNA plasmid (i.e. a polycistronic DNA plasmid of the disclosure), but it is understood that the discussion thereof applies also to other vectors, e.g. viral vectors.

Polycistronic plasmids are known in the art, hence, the skilled person is able to design and construct the polycistronic plasmid of the disclosure.

In preferred embodiments, the polycistronic plasmid of the disclosure comprises one or more co-expression elements, i.e. nucleic acid sequences which allow co-expression of the polypeptide and the one or more immunoinhibitory compounds from the plasmid as separate molecules.

In some embodiments of the present disclosure, the polycistronic plasmid comprises a co-expression element, which causes that the polypeptide and the one or more immunoinhibitory compounds are transcribed on a single transcript but independently translated into the polypeptide and the one or more immunoinhibitory compounds. Hence, the presence of the co-expression element results in a final production of separate translation products.

In some embodiments, such co-expression element is an IRES element (internal ribosome entry site). In others embodiment, such co-expression element is a 2A self-cleaving peptide (2A peptide). Both co-expression elements are known in the art.

If more than one immunoinhibitory compound is expressed from the polycistronic plasmid of the disclosure, an IRES element and/or 2A peptide needs to be present in plasmid, e.g. upstream of each nucleic acid sequence encoding an immunoinhibitory compound.

In other embodiments, the polycistronic plasmid comprises a co-expression element which causes that the polypeptide and the one or more immunoinhibitory compounds are transcribed as separate transcripts, which results in separate transcription products and thus separate proteins.

In some embodiments such co-expression element is a bidirectional promoter.

In other embodiments, such co-expression elements are various promotors, i.e. the polycistronic plasmid comprises a promoter for each of the nucleotide sequences encoding either the polypeptide or the one or more immunoinhibitory compounds. Both co-expression elements are known in the art.

The above-described co-expression elements can be combined in any manner, i.e. the polycistronic plasmid of the disclosure may comprise one or several of such same or different co-expression elements.

### Immunoinhibitory Compounds

The polycistronic plasmid of the present disclosure comprises one or more nucleic acid sequences encoding one or more immunoinhibitory compounds.

In some embodiments of the present disclosure, the immunoinhibitory compound is a compound that is known to induce, increase or maintain immune tolerance.

In some embodiments of the present disclosure, the immunoinhibitory compound is an extracellular part of inhibitory checkpoint molecules. In some embodiments, the inhibitory checkpoint molecule is selected from the group consisting of CLTA-4 (SEQ ID NO: 72), PD-1 (SEQ ID NO: 74), BTLA and TIM-3. In some embodiments, the inhibitory checkpoint molecule is CLTA-4 (SEQ ID NO: 72). In some embodiments, the inhibitory checkpoint molecule is PD-1 (SEQ ID NO: 74). In some embodiments, the inhibitory checkpoint molecule is BTLA. In some embodiments, the inhibitory checkpoint molecule is TIM-3. In some embodiments of the present disclosure, the immunoinhibitory compound is a cytokine selected from the group consisting of IL-10 (SEQ ID NO: 66), TGFβ1 (SEQ ID NO: 60), TGFβ2 (SEQ ID NO: 62), TGFβ3 (SEQ ID NO: 64), IL-27, IL-2, IL-37 and IL-35. In some embodiments, the cytokine is IL-10 (SEQ ID NO: 66). In some embodiments, the cytokine is TGFβ1 (SEQ ID NO: 60). In some embodiments, the cytokine is TGFβ2 (SEQ ID NO: 62). In some embodiments, the cytokine is TGFβ3 (SEQ ID NO: 64). In some embodiments, the cytokine is IL-27. In some embodiments, the cytokine is IL-2. In some embodiments, the cytokine is IL-37. In some embodiments, the cytokine is IL-35.

In some embodiments of the present disclosure, the DNA plasmid comprises nucleic acid sequences encoding 2, 3, 4, 5, 6, 7 or 8 immunoinhibitory compounds. In preferred embodiments, the DNA plasmid comprises nucleic acid sequences encoding 2 to 6 immunoinhibitory compounds, e.g. 2 or 3 or 4 or 5 or 6 different immunoinhibitory compounds. The immunoinhibitory compounds may be the same or different, preferably different.

In preferred embodiments, the different immunoinhibitory compounds generate or promote a tolerance-inducing environment on many different levels. By way of example, the plasmid of the disclosure may comprise nucleic acid sequences encoding 3 different immunoinhibitory compounds, wherein the first induces tolerance, the second increases tolerance and the third maintains tolerance.

### Pharmaceutical Compositions

The construct of the disclosure may be administered to a subject as a pharmaceutical composition comprising the construct, e.g. the form of a polynucleotide or multimeric protein, such as dimeric protein, and a pharmaceutically acceptable carrier.

The construct of the disclosure may be administered to a subject as a pharmaceutical composition comprising the construct, e.g. the form of a polynucleotide or multimeric protein and a pharmaceutically acceptable carrier.

The construct of the disclosure may be administered to a subject as a pharmaceutical composition comprising the construct, e.g. the form of a polynucleotide or dimeric protein and a pharmaceutically acceptable carrier.

A further aspect of the disclosure is pharmaceutical composition comprising a pharmaceutically acceptable carrier and
i) a polypeptide, the polypeptide comprising, in the specified order:
a. a first targeting unit, a first joint region;
b. an antigenic unit;
c. a second joint region; and
d. a second targeting unit;
   wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii), such a dimeric protein, consisting of two polypeptides as defined in ii).

A further aspect of the disclosure is pharmaceutical composition comprising a pharmaceutically acceptable carrier and:
i) a polypeptide, the polypeptide comprising, in the specified order:
a. a first targeting unit, a first joint region;
b. an antigenic unit;
c. a second joint region; and
d. a second targeting unit;
   wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii).

A further aspect of the disclosure is a pharmaceutical composition comprising a pharmaceutically acceptable carrier and:
i) a polypeptide, the polypeptide comprising, in the specified order:
a. a first targeting unit, a first joint region;
b. an antigenic unit;
c. a second joint region; and
d. a second targeting unit;
   wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides as defined in ii). Suitable pharmaceutically acceptable carriers include, but are not limited to, saline, buffered saline, such as PBS, dextrose, water, glycerol, ethanol, sterile isotonic aqueous buffers, and combinations thereof.

In some embodiments, the pharmaceutically acceptable carrier or diluent is an aqueous buffer. In other embodiments, the aqueous buffer is Tyrode's buffer, e.g. Tyrode's buffer comprising 140 mM NaCl, 6 mM KCl, 3 mM CaCl2, 2 mM MgCl2, 10 mM 4-(2-hydroxyethyl)-1-piperazineethanesulfonic acid (Hepes) pH 7.4 and 10 mM glucose.

Suitable adjuvants may include, but are not limited to, dexamethasone, B subunits of enterotoxin cholera toxin (CTB), TLR2 ligands, helminth-derived excretory/secretory (ES) products, rapamycin, or vitamin D3 analogues and aryl hydrocarbon receptor ligands.

In some specific embodiments the composition may comprise a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO—PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO) 4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In some embodiments, the composition comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/V, 0.2% w/v to 8% w/v, 0.2% w/v to 6% w/v, 0.2% w/v to 4% w/v, 0.4% w/v to 18% w/V, 0.6% w/v to 18% w/v, 0.8% w/v to 18% w/v, 1% w/v to 18% w/v, 2% w/v to 18% w/V, 1% w/v to 5% w/v, or 2% w/v to 4% w/v. Particularly preferred are amounts in the range of from 0.5% w/v to 5% w/v. In other embodiments, the composition comprises the amphiphilic block co-polymer in an amount of from 2% w/v to 5% w/v, such as about 3% w/v. For pharmaceutical compositions comprising polynucleotides, the compositions may further comprise molecules that ease transfection of cells.

The pharmaceutical composition may be formulated in any way suitable for administration to a subject, e.g. a patient suffering or suspected of suffering from autoimmune diseases, allergic diseases or graft rejection, e.g. for intradermal or intramuscular injection.

The pharmaceutical composition, comprising in some embodiments a polynucleotide as described herein, e.g. comprised in a vector such as a polycistronic vector, may be administered in any way suitable for administration to a subject, such as administered by intradermal, intramuscular, or subcutaneous injection, or by mucosal or epithelial application, such as intranasal or oral administration.

In preferred embodiments, the pharmaceutical composition comprises a polynucleotide as described herein, e.g. comprised in a vector such as a polycistronic vector, and is administered by intramuscular or intradermal injection.

The pharmaceutical composition of the disclosure typically comprises the polynucleotide in a range of from 0.1 μg to 10 mg, e.g. about 0.2 μg, 0.3 μg, 0.4 μg, 0.5 μg, 0.75 μg, 1 μg, 5 μg, 10 μg, 25 μg, 50 μg, 75 μg, or more; such as from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg. The pharmaceutical composition of the disclosure typically comprises the polypeptide/dimeric protein in the range of from 5 μg to 5 mg.

The amount of polynucleotide/polypeptide/multimeric or dimeric protein may vary depending on whether the pharmaceutical composition is administered for prophylactic or therapeutic treatment, the severity of the immune disease in the individual suffering from it and on parameters like the age, weight, gender, medical history and pre-existing conditions.

### Methods for Preparing the Pharmaceutical Composition

Suitable methods for preparing the pharmaceutical composition or vaccine according to the disclosure are disclosed in WO 2004/076489A1, WO 2011/161244A1, WO 2013/092875A1 and WO 2017/118695A1, which are incorporated herein by reference.

In one aspect, the disclosure relates to a method for preparing a pharmaceutical composition comprising the multimeric protein, for example a dimeric protein, or the polypeptide as defined above by producing the polypeptides in vitro. The in vitro synthesis of the polypeptides and proteins may be carried out by any suitable method known to the person skilled in the art, such as by peptide synthesis or expression of the polypeptide in a variety of expressions systems followed by purification.

Thus, a further aspect of the disclosure is a method for preparing a pharmaceutical composition which comprises a multimeric protein, such a dimeric protein, consisting of multiple polypeptides; or a polypeptide, wherein the method comprises:
a) transfecting cells with a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit; wherein the antigenic unit comprises one or more T cell epitopes of a self-antigen, an allergen, an alloantigen or a xenoantigen;
b) culturing the cells;
c) collecting and purifying the multimeric protein, such as the dimeric protein, or the polypeptide expressed from the cells; and
d) mixing the multimeric protein, such as the dimeric protein, or polypeptide obtained from step c) with a pharmaceutically acceptable carrier.

Thus, a further aspect of the disclosure is a method for preparing a pharmaceutical composition which comprises a multimeric protein consisting of multiple polypeptides; or a polypeptide, wherein the method comprises:
a) transfecting cells with a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit; wherein the antigenic unit comprises one or more T cell epitopes of a self-antigen, an allergen, an alloantigen or a xenoantigen;
b) culturing the cells;
c) collecting and purifying the multimeric protein or the polypeptide expressed from the cells; and
d) mixing the multimeric protein or polypeptide obtained from step c) with a pharmaceutically acceptable carrier.

Thus, a further aspect of the disclosure is a method for preparing a pharmaceutical composition which comprises a dimeric protein consisting of two polypeptides; or a polypeptide, wherein the method comprises:
a) transfecting cells with a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit; wherein the antigenic unit comprises one or more T cell epitopes of a self-antigen, an allergen, an alloantigen or a xenoantigen;
b) culturing the cells;
c) collecting and purifying the dimeric protein or the polypeptide expressed from the cells; and
d) mixing the dimeric protein or polypeptide obtained from step c) with a pharmaceutically acceptable carrier.

In some embodiments, the polynucleotide is comprised in a vector as described herein.

In preferred embodiments, the multimeric protein, such as a dimeric protein, or polypeptide obtained from step c) is dissolved in said pharmaceutically acceptable carrier.

In preferred embodiments, the multimeric protein or polypeptide obtained from step c) is dissolved in said pharmaceutically acceptable carrier

In preferred embodiments, the dimeric protein or polypeptide obtained from step c) is dissolved in said pharmaceutically acceptable carrier. Purification may be carried out according to any suitable method, such as chromatography, centrifugation, or differential solubility.

In another aspect the disclosure relates to a method for preparing a pharmaceutical composition comprising a polynucleotide comprising a nucleotide sequence encoding a polypeptide, the polypeptide comprising, in the specified order,
a. a first targeting unit, a first joint region;
b. an antigenic unit;
c. a second joint region; and
d. a second targeting unit;
   wherein the antigenic unit comprises one or more T cell epitopes of a self-antigen, an allergen, an alloantigen or a xenoantigen, wherein the method comprises:
a) preparing the polynucleotide;
b) optionally cloning the polynucleotide into an expression vector; and
c) mixing the polynucleotide obtained from step a) or the vector obtained from step b) with a pharmaceutically acceptable carrier.

The polynucleotide may be prepared by any suitable method known to the skilled person. For example, the polynucleotide may be prepared by chemical synthesis using an oligonucleotide synthesizer.

The expression vector may be any of the vectors described herein.

In particular, nucleotide sequences encoding the targeting unit and/or the dimerization unit may be synthesized individually and then ligated into a vector backbone to produce the final polynucleotide by ligating into the vector the nucleic acid sequence encoding the antigenic unit.

In one aspect, the disclosure relates to the use of the construct, the polynucleotide, the polypeptide or the multimeric protein, such as the dimeric protein, described herein as a medicament.

In one aspect, the disclosure relates to the use of the construct, the polynucleotide, the polypeptide or the multimeric protein described herein as a medicament.

In one aspect, the disclosure relates to the use of the construct, the polynucleotide, the polypeptide or the dimeric protein described herein as a medicament.

### Medicament

In one aspect, the disclosure relates to the use of the construct, the polynucleotide, the polypeptide, the multimeric protein or the dimeric protein described herein as a medicament.

### Treatment

The construct or pharmaceutical composition of the disclosure may be used to treat autoimmune diseases, allergic diseases or graft rejection, and treatment may either be for prophylactic or for therapeutic purpose.

The construct/pharmaceutical composition is administered such that it induces tolerance in the individual administered with such pharmaceutical composition. Tolerance is induced by either a single administration and preferably by multiple administrations adequately spaced in time.

In a further aspect, the disclosure provides a method for treating a subject having an immune disease selected from the group consisting of autoimmune disease, allergic disease and graft rejection, or being in need of prevention thereof, the method comprising administering to the subject a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii), such as a dimeric protein consisting of two polypeptides as defined in ii).

In a further aspect, the disclosure provides a method for treating a subject having an immune disease selected from the group consisting of autoimmune disease, allergic disease and graft rejection or being in need of prevention thereof, the method comprising administering to the subject a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii).

In a further aspect, the disclosure provides a method for treating a subject having an immune disease selected from the group consisting of autoimmune disease, allergic disease and graft rejection or being in need of prevention thereof, the method comprising administering to the subject a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides as defined in ii).

In yet a further aspect, the disclosure provides a pharmaceutical composition for use in the prophylactic or therapeutic treatment of an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i), such as a dimeric protein consisting of two polypeptides encoded by the nucleotide as defined in i).

In yet a further aspect, the disclosure provides a pharmaceutical composition for use in the prophylactic or therapeutic treatment of an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i).

In yet a further aspect, the disclosure provides a pharmaceutical composition for use in the prophylactic or therapeutic treatment of an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides encoded by the nucleotide as defined in i).

The first and second targeting units, the first and second joint regions, and the antigenic unit are described in detail herein above.

In yet a further aspect, the disclosure provides a pharmaceutical composition for use in the prophylactic or therapeutic treatment of a subject suffering or suspected of suffering from an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i), such as a dimeric protein consisting of two polypeptides encoded by the nucleotide as defined in i),
wherein the pharmaceutical composition is administered to said subject.

In yet a further aspect, the disclosure provides a pharmaceutical composition for use in the prophylactic or therapeutic treatment of a subject suffering or suspected of suffering from an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i),
wherein the pharmaceutical composition is administered to said subject.

In yet a further aspect, the disclosure provides a pharmaceutical composition for use in the prophylactic or therapeutic treatment of a subject suffering or suspected of suffering from an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides encoded by the nucleotide as defined in i),
wherein the pharmaceutical composition is administered to said subject.

The first and second targeting units, the first and second joint regions, and the antigenic unit are described in detail herein above.

In yet a further aspect, the disclosure provides the use of a pharmaceutical composition for the prophylactic or therapeutic treatment of an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i), such as a dimeric protein consisting of two polypeptides encoded by the nucleotide as defined in i).

In yet a further aspect, the disclosure provides the use of a pharmaceutical composition for the prophylactic or therapeutic treatment of an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i).

In yet a further aspect, the disclosure provides the use of a pharmaceutical composition for the prophylactic or therapeutic treatment of an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides encoded by the nucleotide as defined in i).

The first and second targeting units, the first and second joint regions, and the antigenic unit are described in detail herein above.

In yet a further aspect, the disclosure provides the use of a pharmaceutical composition for the manufacture of a medicament for the prophylactic or therapeutic treatment of a subject suffering or suspected of suffering from an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i), such as a dimeric protein consisting of two polypeptides encoded by the nucleotide as defined in i).

In yet a further aspect, the disclosure provides the use of a pharmaceutical composition for the manufacture of a medicament for the prophylactic or therapeutic treatment of a subject suffering or suspected of suffering from an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i).

In yet a further aspect, the disclosure provides the use of a pharmaceutical composition for the manufacture of a medicament for the prophylactic or therapeutic treatment of a subject suffering or suspected of suffering from an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides encoded by the nucleotide as defined in i).

The first and second targeting units, the first and second joint regions, and the antigenic unit are described in detail herein above.

In yet a further aspect, the disclosure provides the use of a pharmaceutical composition for prophylactically or therapeutically treating a subject having an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein, such as a dimeric protein, consisting of multiple polypeptides encoded by the nucleotide as defined in i).

In yet a further aspect, the disclosure provides the use of a pharmaceutical composition for prophylactically or therapeutically treating a subject having an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i).

In yet a further aspect, the disclosure provides the use of a pharmaceutical composition for prophylactically or therapeutically treating a subject having an immune disease selected from the group consisting of autoimmune disease, allergic disease and graft rejection, the pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides encoded by the nucleotide as defined in i).

The first and second targeting units, the first and second joint regions, and the antigenic unit are described in detail herein above.

Further, also disclosed herein is the:

Use of a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i), such as a dimeric protein consisting of two polypeptides encoded by the nucleotide as defined in i),
for the manufacture of a medicament for the prophylactic or therapeutic treatment of a subject having an immune disease selected from the group consisting of autoimmune disease, allergic disease and graft rejection, wherein the medicament is administered to said subject.

Further, also disclosed herein is the:

Use of a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i),
for the prophylactic or therapeutic treatment of subject having an immune disease selected from the group consisting of autoimmune disease, allergic disease and graft rejection, wherein the medicament is administered to said subject.

Further, also disclosed herein is the:

Use of a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides encoded by the nucleotide as defined in i),
for the prophylactic or therapeutic treatment of subject having an immune disease selected from the group consisting of autoimmune disease, allergic disease and graft rejection, wherein the medicament is administered to said subject.

The first and second targeting units, the first and second joint regions, and the antigenic unit are described in detail herein above.

Further, also disclosed herein is:
a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i), such as a dimeric protein consisting of two polypeptides encoded by the nucleotide as defined in i),
when used in the prophylactic or therapeutic treatment of an immune disease selected from the group consisting of autoimmune disease, allergic disease and graft rejection.

Further, also disclosed herein is:
a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i),
when used in the prophylactic or therapeutic treatment of an immune disease selected from the group consisting of autoimmune disease, allergic disease and graft rejection.

Further, also disclosed herein is:
a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of multiple polypeptides encoded by the nucleotide as defined in i),
when used in the prophylactic or therapeutic treatment of an immune disease selected from the group consisting of autoimmune disease, allergic disease and graft rejection.

The first and second targeting units, the first and second joint regions, and the antigenic unit are described in detail herein above.

Further, also disclosed herein is:

A medicament for the prophylactic or therapeutic treatment of subject having an immune disease selected from the group consisting of autoimmune disease, allergic disease and graft rejection by administering to the subject a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i), such as a dimeric protein consisting of two polypeptides encoded by the nucleotide as defined in i).

Also disclosed herein is a medicament for the prophylactic or therapeutic treatment of subject having an immune disease selected from the group consisting of autoimmune disease, allergic disease and graft rejection by administering to the subject a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides encoded by the nucleotide as defined in i).

Also disclosed herein is a medicament for the prophylactic or therapeutic treatment of subject having an immune disease selected from the group consisting of autoimmune disease, allergic disease and graft rejection by administering to the subject a pharmaceutical composition comprising a pharmaceutically acceptable carrier and
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of multiple polypeptides encoded by the nucleotide as defined in i).

The first and second targeting units, the first and second joint regions, and the antigenic unit are described in detail herein above.

Indicators of treatment success are known in the art, including increased levels of antigen-specific regulatory T cells, reduced levels of antigen-specific effector T cells, (and increased levels of regulatory T cells), reduced levels of effector T cells, reduced level of T cell activation in ELISPOT when stimulated with the antigenic unit/T-cell epitopes in the antigenic unit, reduced level of basophil activation in a basophil activation test (BAT). A radioallergosorbent test (RAST) may likewise be used to compare the allergen-specific IgE antibody level in a blood sample from a subject before and after administration of the tolerance-inducing construct, wherein a lower allergen-specific IgE antibody level indicates successful tolerance induction.

### EXAMPLES

### Example 1: Design, Production, and In Vitro Characterization of Tolerance-Inducing Constructs According to the Invention, for Use in the Treatment of Multiple Sclerosis

Myelin oligodendrocyte glycoprotein (MOG) is a protein expressed in the central nervous system. The immunodominant 35-55 epitope of MOG, MOG (35-55), is a primary target for both cellular and humoral immune responses during Multiple sclerosis. MOG (35-55)-induced experimental autoimmune encephalomyelitis (EAE) is the most commonly used animal model of multiple sclerosis (Hunterman, H. et al. 2022).

### Design of DNA Vectors

All gene sequences described were ordered from GenScript (Genscript Biotech B.V., Netherlands) cloned into the expression vector pALD-CV77. DNA vectors were designed, comprising nucleotide sequences encoding the following units/parts:
1. Signal peptide
2. 1^{st} targeting unit
3. 1^{st} joint region: Hinge-region 1 from human IgG3 (SEQ ID NO: 1 amino acids 1-12), Hinge-region 4 from human IgG3 (SEQ ID NO: 1 amino acids 13-27), Glycine-leucine linker (SEQ ID NO: 102)
4. Antigenic unit: MOG (27-63) (SEQ ID NO: 12)
5. 2^{nd} joint region
6. 2^{nd} targeting unit

The differences between the vectors, including the targeting units and the insertion of hinge-region 1 from human IgG3 in the second dimerization unit, are described in Table 1.

| | | 1^{st} Targeting | 2^{nd} Joint | 2^{nd} Targeting |
|---|---|---|---|---|
| Vector ID | Signal peptide | Unit | Region | Unit |
| VB5038* | Murine Ig VH | scFv with with | Hinge | Mature murine |
| (SEQ ID | signal peptide | specificity for | region from | IL10 |
| NO: 24) | (SEQ ID NO: 6) | murine | human | (SEQ ID NO: 9) |
| VB5050 | | CD205 (SEQ | lgG1 (SEQ | |
| (SEQ ID | | ID NO: 7) | ID NO: 8) | |
| NO: 23) | | | | |
| VB5066 | | | | Mature murine |
| (SEQ ID | | | | TGFβ1 |
| NO: 26) | | | | (SEQ ID NO: 10) |
| VB5067 | | | | Mature murine |
| (SEQ ID | | | | CTLA-4*** |
| NO: 28) | | | | (SEQ ID NO: 11) |
| VB5072 | Natural leader | Murine | | Mature murine |
| (SEQ ID | sequence murine | SCGB3A2 | | IL-10 |
| NO: 29) | SCGB3A2 (SEQ | (SEQ ID NO: | | (SEQ ID NO: 9) |
| | ID NO: 15) | 16) | | |
| VB5074 | Natural leader | Murine VSIG- | | |
| (SEQ ID | sequence murine | 3*** (SEQ ID | | |
| NO: 31) | VSIG-3 (SEQ ID | NO: 18) | | |
| | NO: 17) | | | |
| VB5041** | Murine Ig VH | scFv with with | Hinge | Mature murine |
| (SEQ ID | signal peptide | specificity for | region from | IL-10 (SEQ ID |
| NO: 41) | (SEQ ID NO: 6) | murine CD205 | human | NO: 9) |
| VB5042 | | (SEQ ID NO: 7) | IgG1 (SEQ | |
| (SEQ ID | | | ID NO: 8) | |
| NO: 25) | | | Hinge- | |
| VB5043 | | | region 1 | Mature murine |
| (SEQ ID | | | from human | TGFβ1 |
| NO: 27) | | | IgG3 | (SEQ ID NO: 10) |
| | | | (SEQ ID | Mature murine |
| VB5073 | Natural leader | Murine | NO: 19) | IL-10 |
| (SEQ ID | sequence murine | SCGB3A2 | | (SEQ ID NO: 9) |
| NO: 30) | SCGB3A2 (SEQ | (SEQ ID NO: 16) | | |
| | ID NO: 15) | | | |
| VB5075 | Natural leader | Murine VSIG- | | |
| (SEQ ID | sequence murine | 3*** (SEQ ID | | |
| NO: 32) | VSIG-3 (SEQ ID | NO: 18) | | |
| | NO: 17) | | | |

***Extracellular domain

The plasmid DNA vectors VB5038, VB5041, VB5042, VB5043, VB5050, VB5066, VB5067, VB5072, VB5073, VB5074 and VB5075, are vectors according to the disclosure, and encodes tolerance-inducing constructs comprising the targeting units, dimerization units and antigenic units as stated in Table 1.

Vectors used as controls are described in Table 2.

TABLE 2

| Construct | Signal | Targeting | Dimerization | |
|---|---|---|---|---|
| ID | peptide | Unit | unit | Antigenic Unit |
| VB5052 | Natural | Human | Hinge-region 1 | MOG (27- |
| (SEQ ID | leader | CCL3L1 | from human IgG3 | 63) (SEQ ID |
| NO: 33) | sequence | (SEQ ID NO: | (SEQ ID NO: 1 | NO: 12) |
| | human | 21) | amino acids 1-12) | |
| | CCL3L1 | | Hinge-region 4 | |
| | (SEQ ID NO: | | from human IgG3 | |
| | 20) | | (SEQ ID NO: 1 | |
| VB5002b | | | amino acids 13- | MOG (27-63)* |
| (SEQ ID | | | 27) | (SEQ ID NO: |
| NO: 34) | | | Glycine-serine- | 13) |
| | | | linker | |
| | | | (SEQ ID NO: 147) | |
| | | | Human IgG3 CH3 | |
| | | | domain | |
| | | | (SEQ ID NO: 22) | |
| | | | Unit linker: | |
| | | | Glycine-leucine | |
| | | | linker | |
| | | | (SEQ ID NO: 102) | |
| VB5051 | Murine Ig VH | NA | NA | MOG (27-63) |
| (SEQ ID | signal | | | (SEQ ID NO: |
| NO: 35) | peptide | | | 12) |
| | (SEQ ID NO: | | | |
| | 6) | | | |
| VB5001b | Murine Ig VH | NA | NA | MOG (27-63)* |
| (SEQ ID | signal | | | (SEQ ID NO: |
| NO: 36) | peptide | | | 13) |
| | (SEQ ID NO: | | | |
| | 6) | | | |

*The MOG (27-63) sequence was obtained from Krienke et al. 2021. Patent application US2020061166A1.

The DNA vectors VB5052 (SEQ ID NO: 33) and VB5002b (SEQ ID NO: 34) encode fusion proteins comprising a human CCL3L1 targeting unit, which is known to target APCs in an pro-inflammatory manner, i.e. antigen-specific constructs comprising such a targeting unit will induce an inflammatory immune response in subjects to which they are administered and it is expected that this compound induces IFN-γ production (see for instance WO2011161244 A1).

The DNA vectors VB5051 (SEQ ID NO: 35) and VB5001b (SEQ ID NO: 36) encodes the antigenic unit only, MOG (27-63), i.e. a single protein/peptide.

The murine MOG (27-63) antigenic unit comprises the T-cell epitope MOG (35-55).

### In Vitro Characterization of Protein Expression and Secretion of MOG-Containing Constructs

The purpose of this experiment was to characterize protein expression and secretion in the supernatant of mammalian cells transient transfected with MOG containing DNA vectors.

Expi293F cells were obtained from Thermo Fisher Sci. and transiently transfected with the MOG (27-63) containing DNA vectors (VB5042, VB5050, VB5067, VB5072, VB4073, VB5074, and VB5075). Briefly, Expi293F cells (1.7×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). The supernatant was harvested 72 hours post transfection.

HEK293 cells were obtained from ATCC and transiently transfected with the MOG (27-63) containing DNA vector VB5038. Briefly, 2×10⁵ cells/well were plated in 24-well tissue culture plates with 10% FBS growth medium and transfected with 1 μg of the respective DNA vector using Lipofectamine® 2000 reagent under the conditions suggested by the manufacturer (Thermo Fischer Scientific). The transfected cells were maintained at 37° C. with 5% CO₂ for 5 days, and the cell supernatant was collected.

The secreted proteins encoded by the MOG-containing vectors were characterized by sandwich ELISA of the supernatant from transiently transfected Expi293F cells or HEK293 cells, using antibodies against MOG and one of the targeting units. Results are shown in FIG. 4-6.

FIGS. 4A and 4B show that all IL-10-encoding tolerance-inducing constructs were expressed and secreted in vitro in ELISA using mouse anti-MOG antibody as capture antibody (0.25 μg/ml, 100 μl/well, sc-73330, Santa Cruz Biotechnology) and goat anti-murine IL-10 biotinylated antibody as detection antibody (0.8 μg/ml, 100 μl/well, BAF417, R&D Systems).

FIG. 5 shows that the CTLA-4-encoding tolerance-inducing constructs was expressed and secreted in vitro in ELISA using mouse anti-MOG antibody as capture antibody (0.25 μg/ml, 100 μl/well, sc-73330, Santa Cruz Biotechnology) and goat anti-murine CTLA-4 biotinylated antibody as detection antibody (0.8 μg/ml, 100 μl/well, BAF476, R&D Systems).

FIG. 6 shows that the SCGB3A2-encoding tolerance-inducing constructs were expressed and secreted in vitro in ELISA using mouse anti-MOG antibody as capture antibody (0.25 μg/ml, 100 μl/well, sc-73330, Santa Cruz Biotechnology) and goat anti-murine SCGB3A2 as detection antibody (3.3 μg/ml, 100 μl/well, BAF3465, R&D Systems).

The secretion of full-length tolerance-inducing constructs with SCGB3A2 and IL-10 as the first and second targeting units, respectively, was verified by sandwich ELISA of the supernatants using antibodies against murine IL-10 (capture antibody: rat anti-murine IL-10 antibody, 2 μg/ml, 100 μl/well, MAB417, R&D Systems) and murine SCGB3A2 (detection antibody: goat anti-murine SCGB3A2, 3, 3 μg/ml, 100 μl/well, BAF3465, R&D Systems). Results are shown in FIG. 7, and the figure shows that that the vaccines with IL-10 and SCGB3A2 as targeting units encoded by the DNA vectors VB5073 and VB5072, with and without an extra copy of hinge-region 1 from human IgG3 in the second dimerization unit, respectively, were expressed and secreted as full-length fusion proteins.

### In Vitro Characterization of the Binding of the Tolerance-Inducing Constructs to the DEC205 Receptor

The purpose of this experiment was to characterize functional binding of the scFv anti-DEC205 targeting unit to recombinant DEC205 receptor. Functional binding of the targeting unit was assessed in an ELISA on supernatant from HEK293 cells transiently transfected with the DNA vector VB5038 encoding the scFv anti-DEC205 as the first targeting unit, by coating an ELISA-plate with recombinant DEC205 receptor and using antibodies against the antigenic unit or the second targeting unit as detection antibodies.

HEK293 cells were obtained from ATCC and transiently transfected with VB5038. Briefly, 2×10⁵ cells/well were plated in 24-well tissue culture plates with 10% FBS growth medium and transfected with 1 μg of the respective DNA vector using Lipofectamine® 2000 reagent under the conditions suggested by the manufacturer (Invitrogen, Thermo Fischer Scientific). The transfected cells were maintained at 37° C. with 5% CO₂ for 5 days, and the cell supernatant was collected. The secreted protein encoded by VB5038 was assessed in supernatant from transiently transfected cells by direct ELISA. The ELISA plates were coated with 100 μl/well of 5 μg/ml recombinant DEC205 receptor (aa 216-503, OPCD05072, Aviva Systems Biology) and blocked before supernatant was added. Binding of the vaccine protein to the recombinant receptor was detected by antibodies against MOG (mouse anti-MOG antibody, 1 μg/ml, 100 μl/well, sc-73330, Santa Cruz Biotechnology) or murine IL-10 (goat anti-murine IL-10 biotinylated antibody, 1 μg/ml, 100 μl/well, BAF417, R&D Systems).

The results shown in FIG. 8 confirms the binding of the scFv anti-DEC205 containing VB5038 to the DEC205 receptor and the secretion of full-length fusion protein.

### In Vitro Characterization of the Binding of Tolerance-Inducing Constructs to the IL-10 Receptor

The purpose of this experiment was to characterize functional binding of the IL-10 targeting unit to recombinant IL-10 receptor. Functional binding of the targeting unit was assessed in an ELISA on supernatant from HEK293 cells transiently transfected with DNA vaccines encoding IL-10 as the second targeting unit, by coating an ELISA-plate with recombinant IL-10 receptor (IL-10R) and using an antibody against the antigenic unit of the detection antibody.

HEK293 cells were obtained from ATCC and transiently transfected with VB5038. Briefly, 2×10⁵ cells/well were plated in 24-well tissue culture plates with 10% FBS growth medium and transfected with 1 μg of the respective DNA vector using Lipofectamine® 2000 reagent under the conditions suggested by the manufacturer (Invitrogen, Thermo Fischer Scientific). The transfected cells were maintained at 37° C. with 5% CO₂ for 5 days, and the cell supernatant was collected. The secreted proteins encoded by VB5038 was assessed in supernatant from transiently transfected cells by direct ELISA. The ELISA plates were coated with 100 μl/well of 2.5 μg/ml recombinant IL-10 receptor and blocked before supernatant was added. Binding of the vaccine protein to the recombinant receptor was detected by an antibody against MOG (100 μl/well, 1 μg/ml mouse anti-MOG antibody, sc-73330, Santa Cruz Biotechnology).

The results shown in FIG. 9 demonstrate that tolerance-inducing constructs proteins with IL-10 as second targeting unit are able to bind to the IL-10 receptor.

### In Vitro Characterization of the Protein Expression and Secretion Post Transient Transfection of Mammalian Cells of the MOG (27-63) Peptide Encoded in the Vector VB5051

The purpose of this experiment was to evaluate the protein expression and secretion of the MOG (27-63) antigen alone control, encoded by the vector VB5051, in transiently transfected mammalian cells in vitro. Briefly, Expi293F cells (2×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). Supernatants were harvested 72 hours post transfection.

The secretion of the MOG (27-63) peptide was characterized by direct ELISA, coating with the supernatant and detection using an antibody against MOG (capture antibody, 100 μl/well, 3.3 μg/ml mouse anti-MOG antibody, sc-73330, Santa Cruz Biotechnology). FIG. 10 shows that the MOG (27-63) peptide is expressed from VB5051 and secreted from mammalian cells transfected with said vector.

In Vitro Characterization of the Protein Expression and Secretion Post Transient Transfection of Mammalian Cells with the Pro-Inflammatory Control Constructs Encoded by the DNA Vectors VB5052 and VB5002b

The DNA vectors VB5052 (SEQ ID NO: 33) and VB5002b (SEQ ID NO: 34) encode fusion proteins comprising a human CCL3L1 targeting unit known to target APCs in a pro-inflammatory manner, i.e. antigen-specific vaccines comprising such a targeting unit will induce an inflammatory immune response in subjects to which they are administered and it is expected that this compound induces IFN-γ production (see for instance WO2011161244 A1).

The purpose of these experiments was to characterize protein expression and secretion of the proteins encoded by the DNA vectors VB5052 and VB5002b in transiently transfected mammalian cells.

Expi293F cells were obtained from Thermo Fisher Sci. and transiently transfected with the DNA vector VB5052. Briefly, Expi293F cells (1.7×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). Supernatant was harvested 72 hours post transfection.

HEK293 cells were obtained from ATCC and transiently transfected with the DNA vector VB5002b. Briefly, 2×10⁵ cells/well were seeded in 24-well tissue culture plates with 10% FBS growth medium and transfected with 1 μg of the respective DNA vector using Lipofectamine® 2000 reagent under the conditions suggested by the manufacturer (Thermo Fischer Scientific). The transfected cells were incubated at 37° C. with 5% CO₂. Supernatant was harvested 5 days post transfection.

The secreted protein encoded by the DNA vector VB5052 was assessed in supernatant from transiently transfected cells by sandwich ELISA using antibodies against MOG (mouse anti-MOG, 0.25 μg/ml, 100 μl/well, sc-73330, Santa Cruz Biotechnology) and human CCL3L1 (goat anti-human CCL3 0.2 μg/ml, 100 μl/well, BAF270, R&D Systems). The results are shown in FIG. 11A and shows that the pro-inflammatory control vaccine encoded by the vector VB5052 was highly expressed and secreted as a full-length fusion protein.

The secreted protein encoded by the DNA vector VB5002b was assessed in supernatant from transiently transfected cells by sandwich ELISA using antibodies against human IgG3(CH3) (mouse anti-human IgG (CH3 domain), 1 μg/ml, 100 μl/well, 153272, Biorad) and human CCL3L1 (goat anti-human CCL3 0.2 μg/ml, 100 μl/well, BAF270, R&D Systems). The results are shown in FIG. 11B, and shows that the immunogenic control vaccine encoded by the vector VB5002b was expressed and secreted as protein.

Characterization of the Proteins Expressed from the DNA Vectors VB5038, VB5041, VB5042, VB5050, VB5074 and VB5075 by Western Blot

Western blot analysis was performed on supernatant from transfected Expi293F cells to further characterize the proteins encoded by the DNA vectors VB5038, VB5041, VB5042, VB5050, VB5074, VB5075, and VB5002b.

Expi293F cells were obtained from Thermo Fisher Sci. and transiently transfected with the DNA vectors VB5038 and VB5002b. Briefly, Expi293F cells (3×106 cells/ml, 1.6 ml) were seeded in a 6-well culture plate. The cells were transfected with 1 μg/ml plasmid DNA using ExpiFectamine 293 Reagent (Thermo Fisher Sci.), and the plates were incubated on an orbital shaker (19 mm diameter, 125 rpm) in a humidified CO2 cell incubator (8% CO2, 37° C.). After 18 h of incubation, ExpiFectamine 293 Transfection Enhancer (Thermo Fisher Sci.) was added to each well. The plates were incubated for another 78 h before the supernatant was harvested. The samples were prepared by mixing 105 μl supernatant from transfected Expi293F cells with 37.5 μl 4× Laemmli sample buffer (Bio-Rad) with 7.5 μl DTT (Thermo Fisher Sci.) or 7.5 μl ultrapure water for reducing and non-reducing conditions, respectively. The samples (reduced or non-reduced) were heated at 70° C. for 10 minutes before added (added sample volume stated in figure caption) to 4%-20% Criterion TGX Stain-Free precast gels (Bio-Rad). SDS-PAGE was performed in 1× Tris/Glycine/SDS running buffer (Bio-Rad) with a Precision Plus Protein All Blue Prestained protein standard (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with mouse anti-MOG (sc-73330, Santa Cruz Biotechnology) and rat anti murine IL-10 antibody (MAB417, R&D systems) to detect MOG and IL-10, respectively. The membranes were incubated with fluorochrome-conjugated secondary antibodies for 1 h at room temperature, and then washed and dried. Images were acquired by using a ChemiDoc™ MP Imaging System (setting Dylight 488 and 800, Auto Optimal). Results are shown in FIGS. 12A and 12B.

Expi293F cells were obtained from Thermo Fisher Sci. and transiently transfected with the DNA vectors VB5041, VB5042, VB5050, VB5074 and VB5075. Briefly, Expi293F cells (2 or 1.7×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). The plates were incubated for 72 h before the supernatant was harvested. The samples were prepared by mixing 14 μl supernatant from transfected Expi293F cells with 5 μl 4× Laemmli sample buffer (Bio-Rad) with 1 μl DTT (Cayman Chemical) or 1 μl ultrapure water for reducing and non-reducing conditions, respectively (scale-up of total sample volume with the given ratio). The samples (reduced or non-reduced) were heated at 70° C. for 10 minutes before added (added sample volume stated in figure caption) to 4%-20% Criterion TGX Stain-Free precast gels (Bio-Rad). SDS-PAGE was performed in 1× Tris/Glycine/SDS running buffer (Bio-Rad) with a Precision Plus Protein All Blue Prestained protein standard (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with mouse anti-MOG (sc-73330, Santa Cruz Biotechnology) or rat anti-murine IL-10 (MAB417, R&D Systems) to detect MOG or IL-10, respectively. The membranes were incubated with fluorochrome-conjugated species-specific secondary antibodies for 1 h at room temperature, and then washed and dried. For IL10 detection in the Dylight-488 channel, membranes were re-probed with Dylight-488 secondary antibody. Membranes were reactivated in ethanol and TBST. Membranes were blocked, incubated with Dylight 488-conjugated secondary antibodies for 1 h at room temperature, and then washed and dried. Images were acquired by using a ChemiDoc™ MP Imaging System. Results are shown in FIGS. 12C, 12D, 12E, 12F and 12G.

The western blot analysis with anti-MOG antibody of the vaccines (VB5038, VB5041, VB5042 and VB5050) encoding scFv anti-DEC205 as the first targeting unit, MOG (27-63) as the antigenic unit, and IL-10 as the second targeting unit (FIGS. 12A and C), show that theses vaccines were secreted as full-length fusion proteins. Detection with anti-murine IL-10 antibody (FIGS. 12B and D) shows a single band at the same molecular as for the previously described anti-murine MOG antibody, demonstrating that both antibodies detected the same protein band, and, thus, confirming that MOG and IL-10 are parts of the same fusion protein. The non-reduced samples in FIGS. 12B and E show dimerization of these proteins.

The western blot analysis with anti-murine MOG of the vaccines encoding VSIG-3 as the first targeting unit, MOG (27-63) as the antigenic unit, and IL-10 as the second targeting unit (VB5074 and VB5075) (FIG. 12F), shows that theses vaccines were secreted as full-length fusion proteins. The proteins migrated at a slower rate than expected based on their calculated molecular weight, which can be explained by known posttranslational glycosylation's (FIG. 12F). Detection with anti-murine IL-10 on supernatant from cells transfected with VB5074 and VB5075 (FIG. 12G) shows that both antibodies detected the same protein band, thus confirming that MOG and IL-10 are parts of the same fusion protein.

### Example 2: Assessment of Tolerance Inducing Ability of VB5067

The tolerance-inducing ability of VB5067 (described in Table 1) was assessed in spleens from mice vaccinated once with 50 μg of VB5067 and determined by calculating the IL-10/IFN-γ ratio induced. The IL-10 (an anti-inflammatory cytokine known to exert immunosuppressive functions) and IFN-γ (a marker for inducing an inflammatory immune response) signals were determined in a dual color FluoroSpot assay following restimulation of splenocytes harvested from vaccinated mice with MOG (35-55) peptide. The IL-10/IFN-γ ratio indicates to which extent the immune responses induced by the DNA vectors are skewed towards a tolerogenic response. A tolerogenic profile was further assessed by the frequencies of MOG (38-49)-specific Foxp3+ T cells induced in response to vaccination and detected ex vivo. Foxp3 acts as a master regulator of the immune suppressive pathway in the development and function of regulatory T cells (Tregs) and indicate Treg cells that act to suppress and control MOG-specific inflammatory immune responses, thereby maintaining self-tolerance. The results obtained were compared to the responses elicited by the pro-inflammatory control vaccine VB5052 or and the tolerance-inducing ability of VB5051 vaccination (both described in Table 2).

### Mouse Vaccination and Fluorospot

The following study design was applied:

Female, 6-week-old C57BL/6 mice were obtained from Janvier Labs (France). All animals were housed in the animal facility at the Radium Hospital (Oslo, Norway). All animal protocols were approved by the Norwegian Food Safety Authority (Oslo, Norway). 5 mice/group were used for the testing of VB5067 (described in Table 1), VB5052 and VB5051 (described in Table 2). VB5052 was included as a pro-inflammatory version of a MOG (27-63) encoding vaccine. VB5052 comprise a human CCL3L1 targeting unit known to target APCs in a pro-inflammatory manner, i.e. a vaccine comprising such a targeting unit will induce an inflammatory immune response in subjects to which they are administered and it is expected that this compound induces IFN-γ production. A DNA vector encoding the MOG (27-63) peptide alone, VB5051, was included as a comparison to VB5067.

One dose of 50 μg of the DNA vector VB5067 or the control vectors VB5051 or VB5052 dissolved in sterile PBS was administered by intramuscularly needle injection to each tibialis anterior (2×25 μl, 1000 μg/ml) followed by electroporation with AgilePulse in vivo electroporation system (BTX, USA). The spleens were harvested 7 days after vaccination and mashed in a cell strainer to obtain single cell suspensions. The red blood cells were lysed using ammonium-chloride-potassium (ACK) lysing buffer. After washing, the splenocytes were counted using the NucleoCounter NC-202 (ChemoMetec, Denmark), resuspended to a final concentration of 6×106 cells/ml and seeded as 6×105 cells/well in a 96-well IFN-γ/IL-10 dual color FluoroSpot plate. The splenocytes were then restimulated for 44 h with 16.67 μg/ml MOG (35-55) peptide before tested for IFN-γ and IL-10 cytokine production in a dual color FluoroSpot assay according to the manufacturer's protocol (Mabtech AB, Sweden). Spot-forming cells were measured in an IRIS Fluorospot and ELISpot plate reader (Mabtech AB) and analyzed using the Apex software (Mabtech AB). Results are shown as the mean number of triplicates of IL-10+ or IFN-γ+ spots/106 splenocytes.

As can be seen from FIG. 13A, production of IL-10 was detected in non-restimulated splenocytes harvested from mice vaccinated with all three constructs; VB5067, VB5051 and VB5052, while only low background levels of IFN-γ were observed. Upon MOG (35-55) restimulation of splenocytes, elevated levels of IFN-γ were detected after vaccination with VB5052 that was significantly increased above those elicited by VB5067 and VB5051, as shown in FIG. 13B. To avoid excess inflammation and assure eventual resolution of inflammation, it is important that the production of pro-inflammatory cytokines, such as IFN-γ, is regulated by negative feedback mechanisms including the production of anti-inflammatory cytokines such as IL-101. Therefore, the increased level of IL-10 observed in response to VB5052 may be explained by such a feedback mechanism to control the inflammatory response induced. As shown in FIG. 13C, a significantly higher IL-10/IFN-γ ratio was detected for VB5067 compared to VB5052, indicating a higher immunosuppressive potential of VB5067 compared to VB5052.

Flow Cytometry Analysis of MOG (38-49)-Specific T Cells in Spleens from Vaccinated Mice Using the Tetramer (H-2IAb/GWYRSPFSRVVH)

The generation of MOG-specific Foxp3+ cells, i.e. indicating T cells that act to suppress and control MOG-specific inflammatory immune responses, and thereby maintaining self-tolerance, was identified in mouse by MOG-specific tetramer staining and flow cytometry (CD4+MOG (38-49)-tet+Foxp3+ cells).

Briefly, 2×10⁶ splenocytes pooled from each group were transferred to 96 well V bottom plate. Tetramers and antibodies were diluted in PBS with 5% FBS before use and protected from light. All steps that required cell wash were performed with PBS with 5% FBS unless otherwise stated. First, the cells were stained with ProT2® MHC Class II Tetramers specific for (MOG 38-49) (1 μg/ml, H-2 IAb-GWYRSPFSRVVH-ProT2® Tetramer PE, 2958, Proimmune) and the plates were incubated in a humidified CO2 cell incubator (5% CO2, 37° C.) for 2 h. Without washing the cells, FC receptors were blocked on ice for 5 min to precvent non-specific binding of flowcytometry antibodies to the Fc receptor (0.25 μg/ml, TruStain FcX™ PLUS (anti-mouse CD16/32) Antibody, 156604, Biolegend). Without washing the cells, the cells were stained 30 min on ice with surface antibody cocktail containing anti-mouse CD8 PE-Cy7 (0.25 μg/ml, Clone: 53-6.7, 100721, BD Biosciences), anti-mouse CD4 eFluor450 (0.25 μg/ml, Clone: GK1.5, 48-0041-82, Thermofischer/eBioscience), anti-mouse CD25 PerCP-Cy5.5 (0.25 μg/ml, Clone: PC61, 102030, Biolegend). The cells were washed twice with PBS. Next, the cells were stained on ice for 10 min with fixable viability dye (150 μl per well, 1:8000 dilution in PBS, Fixable Viability Stain 780, 565388, BD biosciences). The cells were washed twice with only PBS and fixed and permeabilized using Foxp3/Transcription Factor Staining Buffer Set according to the manufacturer's instruction (200 μl per well, 00-5523-00, Thermofischer/eBioscience). The cells were washed and stained for 30 min on ice with intracellular antibody cocktail containing anti-mouse FOXP3 eFluor 660 (0.25 μg/ml, Clone: FJK-16s, 50-5773-82, Thermofischer/eBioscience), anti-mouse Ki-67 Alexa Fluor 488 (0.25 μg/ml, Clone: Clone: 11F6, 151204, Biolegend). The cells were washed and resuspended in 150 μl of PBS with 5% FBS and analyzed with BD FACSymphony™ A3 Cell Analyzer. The following controls were used as a guide for gating desired population using FlowJo™ v10.8 Software (BD Life Sciences), Unstained controls (=cells did not receive any antibody) and Fluorescence Minus One (FMO) controls (=samples stained with all the fluorophores labelled antibodies, minus one to accurately discriminating positive versus negative signals).

As shown in FIG. 14, a higher percentage of MOG (38-49)-specific Foxp3+ cells was detected in response to VB5067 compared to VB5051.

Example 2 thus shows that vaccination with VB5067, encoding a construct with scFv-anti DEC205 and CTLA-4 as targeting units and MOG (27-63) as antigen unit, results in a higher anti-inflammatory to inflammatory cytokine ratio (IL-10/IFN-γ) and a lack of inflammatory IFN-γ production compared to the pro-inflammatory vaccine VB5052. Moreover, the scFv anti-DEC205 and CTLA-4 targeted protein induces a higher percentage of MOG (38-49)-specific Foxp3+ cells compared to VB5051. All together, these results indicate that vaccination with the scFv anti-DEC205 and CTLA-4 bi-specific construct exhibits a lack of pro-inflammatory cytokine production (IFN-γ), as opposed to VB5052, and elicits a greater antigen-specific tolerogenic response compared to VB5051.
¹ Sugimoto M A, Sousa L P, Pinho V, Perretti M, Teixeira M M. Resolution of Inflammation: What Controls Its Onset? Front Immunol. 2016 Apr. 26; 7:160.doi: 10.3389/fimmu.2016.00160.

### Example 3: Assessment of Tolerance-Inducing Ability of VB5042

The tolerance-inducing ability of VB5042 (described in Table 1) was determined and compared to the responses induced by the pro-inflammatory control vaccine of VB5052 (described in Table 2) and the tolerance-inducing ability of VB5051 (described in Table 2), as described in Example 2.

As can be seen from FIG. 15A, production of IL-10 was detected in non-restimulated splenocytes harvested from mice vaccinated with all three constructs; VB5042, VB5051 and VB5052, while only low background levels of IFN-γ were observed. As shown in FIG. 15B, upon MOG (35-55) restimulation of splenocytes, elevated levels of IFN-γ were detected after vaccination with VB5052, which were significantly increased above those elicited by VB5042 and VB5051. The increased level of IL-10 observed in response to VB5052 may be explained by a potential feedback mechanism to control the inflammatory response, as described in Example 2. Splenocytes from mice vaccinated with either VB5042 and VB5051 showed similar levels of IL-10 and IFN-γ both with (FIG. 15A) and without (FIG. 15B) MOG (35-55) peptide re-stimulation. As shown in FIG. 15C, a significantly higher IL-10/IFN-γ ratio was detected for VB5042 compared to VB5052, indicating a higher immunosuppressive potential of VB5042 compared to VB5052.

As shown in FIG. 16, a higher percentage of MOG (38-49)-specific Foxp3+ cells was detected in response to VB5042 compared to VB5051.

Example 3 thus shows that vaccination with VB5042, encoding a construct with scFv anti-DEC205 and IL-10 as targeting units and MOG (27-63) as antigenic unit, results in a higher non-inflammatory to inflammatory cytokine ratio (IL-10/IFN-γ) and a lack of inflammatory IFN-γ, compared to the pro-inflammatory vaccine version VB5052. Moreover, the scFv anti-DEC205 and IL-10 targeted protein induced a higher frequency of MOG (38-49)-specific Foxp3+ cells compared to VB5051. All together, these results indicate that vaccination with the scFv anti-DEC205 and IL-10 bi-specific construct exhibits a lack of pro-inflammatory cytokine production (IFN-γ), as opposed to VB5052, and elicits a greater antigen-specific tolerogenic response compared to VB5051.

### Example 4: Assessment of Tolerance-Inducing Ability of VB5073

The tolerance-inducing ability of VB5073 (described in Table 1) was determined and compared to the responses induced by the pro-inflammatory control vaccine VB5052 (described in Table 2) and the tolerance-inducing ability of VB5051 (described in Table 2), as described in Example 2.

As can be seen from FIG. 17A, production of IL-10 was detected in non-restimulated splenocytes harvested from mice vaccinated with all the three constructs; VB5073, VB5051 and VB5052, while only low background levels of IFN-γ were observed. As shown in FIG. 17B, upon MOG (35-55) restimulation of the splenocytes, elevated levels of IFN-γ were detected after vaccination with VB5052, which were significantly increased above those elicited by VB5073 and VB5051. The increased level of IL-10 observed in response to VB5052 may be explained by a potential feedback mechanism to control the inflammatory response, as described in Example 2. The splenocytes from the mice vaccinated with either VB5073 or VB5051 showed similar levels of IL-10 and IFN-γ both with (FIG. 17B) and without (FIG. 17A) MOG (35-55) peptide re-stimulation. As shown in FIG. 17C, a significantly higher IL-10/IFN-γ ratio was detected for VB5073 compared to VB5052, indicating a higher immunosuppressive potential of VB5073 compared to VB5052.

As shown in FIG. 18, a higher percentage of MOG (38-49)-specific Foxp3+ cells was detected in response to VB5073 compared to VB5051.

Example 4 thus shows that vaccination with VB5073, encoding a construct with SCGB3A2 and IL-10 as targeting units and MOG (38-49) as antigenic unit, results in a higher non-inflammatory to inflammatory cytokine ratio (IL-10/IFN-γ), shows a lack of inflammatory IFN-γ production compared to the pro-inflammatory construct VB5052, and induces a higher frequency of MOG (38-49)-specific Foxp3+ cells compared to VB5051. All together, these results indicate that vaccination with the SCGB3A2 and IL-10 bi-specific construct exhibits a lack of pro-inflammatory cytokine production (IFN-γ), as opposed to VB5052, and elicits a greater antigen-specific tolerogenic response compared to VB5051.

### Example 5: Design, Production, and In Vitro Characterization of Tolerance-Inducing Constructs According to the Invention—with Six T-Cell Epitopes for Use in the Treatment of Shellfish Allergy

Tropomyosin is the major allergen in shellfish. Six major T-cell epitopes were identified for tropomyosin from the species Metapenaeus ensis (Met e 1) in a Balb/c mouse model of Met e 1 hypersensitivity. Oral immunotherapy with peptides of the six T-cell epitopes effectively reduced allergic responses towards shrimp tropomyosin (Wai, C. Y. Y et al. 2015).

### Design of DNA Vectors

The DNA vectors VB5077 and VB5078 were designed and produced, comprising nucleic acid sequences encoding the elements/units listed in Table 3 below. All gene sequences described were ordered from GenScript (Genscript Biotech B.V., Netherlands) cloned into the expression vector pALD-CV77.

TABLE 3

| | Vector ID | |
|---|---|---|
| | VB5077 | VB5078 |
| | (SEQ ID NO: 37) | (SEQ ID NO: 38) |
| Signal peptide | Murine Ig VH signal peptide (SEQ ID NO: 6) | |
| 1^{st} targeting unit | scFv with with specificity for murine CD205 | |
| | (SEQ ID NO: 7) | |
| 1^{st} dimerization | Hinge-region 1 from human IgG3, Hinge-region 4 | |
| unit: | from human IgG3 (SEQ ID NO: 1) | |
| Antigenic unit: | Met e 1 (241-260), (210-230), (136-155), (76-95), | |
| | (46-65), (16-35) (SEQ ID NO: 22) | |
| 2^{nd} dimerization | Hinge region | Hinge region |
| unit | form human IgG1 | form human IgG1 |
| | (SEQ ID NO: 8) | (SEQ ID NO: 8) |
| | | Hinge-region 1 |
| | | from human IgG3 |
| | | (SEQ ID NO: 19) |

| | |
|---|---|
| 2^{nd} targeting unit | Murine IL-10 (SEQ ID NO: 9) |

The DNA vectors VB5077 (SEQ ID NO: 37) and VB5078 (SEQ ID NO: 38), vectors according to the disclosure, encodes constructs comprising the targeting units, dimerization units and antigenic unit as stated in the table above.

The Met e 1 (241-260), (210-230), (136-155), (76-95), (46-65), (16-35) antigenic unit (SEQ ID NO: 22) contains GGGGSGGGGS (SEQ ID NO: 80) linker between the T cell epitopes.

### In Vitro Characterization of Protein Expression and Secretion of Met e 1-Containing Tolerance-Inducing Constructs

The purpose of this experiment was to characterize expression and secretion of proteins encoded by the Met e 1-containing DNA vectors VB5077 and VB5078 post transient transfection of mammalian cells.

Expi293F cells were obtained from Thermo Fisher Sci. and transiently transfected with the DNA vectors VB5077 and VB5078. Briefly, Expi293F cells (1.7×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). The plates were incubated for 72 h before the supernatant was harvested.

The secreted proteins encoded by the Met e 1 containing vectors were assessed in supernatant from transiently transfected cells by sandwich ELISA using antibodies against murine IL-10 (capture antibody: mouse anti-murine IL-10 antibody, 2 μg/ml, 100 μl/well, MAB417, R&D Systems, detection antibody: goat anti-murine IL-10 biotinylated antibody, 0.8 μg/ml, 100 μl/well, BAF417, R&D Systems). Results are shown in FIG. 19, and shows that both Met e 1-containing constructs were expressed and secreted at high levels

Characterization of the Intact Proteins Expressed from VB5077 and VB5078

Western blot analysis was performed on supernatant samples from transfected Expi293F cells to further characterize the proteins encoded by VB5077 and VB5078.

The samples were prepared by mixing 14 μl supernatant from transfected Expi293F cells with 5 μl 4× Laemmli sample buffer (Bio-Rad) with 1 μl DTT (Cayman Chemical) or 1 μl ultrapure water for reducing and non-reducing conditions, respectively (scale-up of total sample volume with the given ratio). The samples (reduced or non-reduced) were heated at 70° C. for 10 minutes before added (added sample volume stated in figure caption) to 4%-20% Criterion TGX Stain-Free precast gels (Bio-Rad). SDS-PAGE was performed in 1× Tris/Glycine/SDS running buffer (Bio-Rad) with a Precision Plus Protein All Blue Prestained protein standard (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with rat anti-murine IL-10 (MAB417, R&D Systems) to detect IL-10. The membranes were incubated with fluorochrome-conjugated species-specific secondary antibody for 1 h at room temperature, and then washed and dried. Images were acquired by using a ChemiDoc™ MP Imaging System.

Results are shown in FIG. 20.

The western blot analysis with anti-murine IL-10 antibody shows that the six Met e 1 T-cell epitope containing constructs were secreted as full-length fusion proteins.

Sequences

SEQ ID NO: 1
Amino acid sequence of hinge exon h1 from IgG3 (amino acids 1-12) and hinge exon
h4 (amino acids 13-27) from human IgG3
E¹LKTPLGDTTHT¹²E¹³PKSCDTPPPCPRCP²⁷
SEQ ID NO: 2
Amino acid sequence of hinge regions of human IgG1: upper hinge region (amino
acids 1-4), middle hinge region (amino acids 5-15) and lower hinge region (amino acids
16-23).
E¹PKS⁴C⁵DKTHTCPPCP¹⁵A¹⁶PELLGGP²³
SEQ ID NO: 3
Amino acid sequence of the CH3 domain of human IgG3
GQPREPQVYTLPPSREEMTKNQVSLTCLVKGFYPSDIAVEWESSGQPENNYNTTPP
MLDSDGSFFLYSKLTVDKSRWQQGNIFSCSVMHEALHNRFTQKSLSLSPGK
SEQ ID NO: 4
Amino acid sequence of the CH3 domain of human IgG1
GQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 5
Amino acid sequence of the CREB bZIP motif
VKCLENRVAVLENQNKTLIEELKALKDLY
SEQ ID NO: 6
Mouse immunoglobulin heavy chain signal sequence (Ig VH signal seq)
MNFGLRLIFLVLTLKGVQC
SEQ ID NO: 7
Mouse single chain variable fragment (scFv) anti-DEC205
DIQMTQSPSFLSTSLGNSITITCHASQNIKGWLAWYQQKSGNAPQLLIYKASSLQSGV
PSRFSGSGSGTDYIFTISNLQPEDIATYYCQHYQSFPWTFGGGTKLELKGGGGSGGG
GSGGGGSEVKLLESGGGLVQPGGSLRLSCAASGFTFNDFYMNWIRQPPGQAPEWL
GVIRNKGNGYTTEVNTSVKGRFTISRDNTQNILYLQMNSLRAEDTAIYYCARGGPYYY
SGDDAPYWGQGVMVTVSS
SEQ ID NO: 8
Hinge region form human IgG1. Upper hinge region hIgG1 (1-5), Middle hinge region
hIgG1(6-20)
GLQGLEPKSCDKTHTCPPCP
SEQ ID NO: 9
Murine IL-10
SRGQYSREDNNCTHFPVGQSHMLLELRTAFSQVKTFFQTKDQLDNILLTDSLMQDFK
GYLGCQALSEMIQFYLVEVMPQAEKHGPEIKEHLNSLGEKLKTLRMRLRRCHRFLPC
ENKSKAVEQVKSDFNKLQDQGVYKAMNEFDIFINCIEAYMMIKMKS
SEQ ID NO: 10
Mature murine TGFβ1
ALDTNYCFSSTEKNCCVRQLYIDFRKDLGWKWIHEPKGYHANFCLGPCPYIWSLDTQ
YSKVLALYNQHNPGASASPCCVPQALEPLPIVYYVGRKPKVEQLSNMIVRSCKCS
SEQ ID NO: 11
Murine CTLA-4 extracellular domain
EAIQVTQPSVVLASSHGVASFPCEYSPSHNTDEVRVTVLRQTNDQMTEVCATTFTEK
NTVGFLDYPFCSGTFNESRVNLTIQGLRAVDTGLYLCKVELMYPPPYFVGMGNGTQIY
VIDPEPCPDSD
SEQ ID NO: 15
Murine MARCO ligand SCGB3A2 signal sequence
MKLVSIFLLVTIGICGYSATA
SEQ ID NO: 16
Murine MARCO ligand SCGB3A2
LLINRLPVVDKLPVPLDDIIPSFDPLKMLLKTLGISVEHLVTGLKKCVDELGPEASEAVKK
LLEALSHLV
SEQ ID NO: 17
Murine VISTA ligand VSIG-3 signal sequence
MTRRRSAPASWLLVSLLGVATS
SEQ ID NO: 18
Murine VISTA ligand VSIG-3 extracellular domain
LEVSESPGSVQVARGQTAVLPCAFSTSAALLNLNVIWMVIPLSNANQPEQVILYQGGQ
MFDGALRFHGRVGFTGTMPATNVSIFINNTQLSDTGTYQCLVNNLPDRGGRNIGVTG
LTVLVPPSAPQCQIQGSQDLGSDVILLCSSEEGIPRPTYLWEKLDNTLKLPPTATQDQV
QGTVTIRNISALSSGLYQCVASNAIGTSTCLLDLQVISPQPRSV
SEQ ID NO: 19
Hinge h1 hIgG3
ELKTPLGDTTHT
SEQ ID NO: 20
Human CCL3L1 signal sequence
MQVSTAALAVLLCTMALCNQVLS
SEQ ID NO: 21
Human CCL3L1
APLAADTPTACCFSYTSRQIPQNFIADYFETSSQCSKPSVIFLTKRGRQVCADPSEEW
VQKYVSDLELSA
SEQ ID NO: 23
Amino acid sequence of VB5050. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), MOG
amino acids 27-63 (298-334), Upper hinge region hIgG1 (335-339), Middle hinge
region hIgG1 (340-354), Murine IL-10 (355-514).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLSPGKNATGMEVGWYRSPFSRVVHLYRNGKDQDAEQAPGLQGLE
PKSCDKTHTCPPCPSRGQYSREDNNCTHFPVGQSHMLLELRTAFSQVKTFFQTKDQ
LDNILLTDSLMQDFKGYLGCQALSEMIQFYLVEVMPQAEKHGPEIKEHLNSLGEKLKTL
RMRLRRCHRFLPCENKSKAVEQVKSDFNKLQDQGVYKAMNEFDIFINCIEAYMMIKM
KS
SEQ ID NO: 24
Amino acid sequence of VB5038. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), MOG
amino acids 27-63 (298-334), Upper hinge region hIgG1 (335-339), Middle hinge
region hIgG1 (340-354), Murine IL-10 (355-514). Parts of MOG(27-63) sequence was
obtained from the article Krienke et al. 2021.
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLSPGKNATGMEVGWYRSPFSRVVHLYRNGKDQDAEAQPGLQGLE
PKSCDKTHTCPPCPSRGQYSREDNNCTHFPVGQSHMLLELRTAFSQVKTFFQTKDQ
LDNILLTDSLMQDFKGYLGCQALSEMIQFYLVEVMPQAEKHGPEIKEHLNSLGEKLKTL
RMRLRRCHRFLPCENKSKAVEQVKSDFNKLQDQGVYKAMNEFDIFINCIEAYMMIKM
KS
SEQ ID NO: 25
Amino acid sequence of VB5042. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), MOG
amino acids 27-63 (298-334), Upper hinge region hIgG1 (335-339), Middle hinge
region hIgG1 (340-354), Hinge h1 hIgG3 (355-366), Murine IL-10 (367-526).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLSPGKNATGMEVGWYRSPFSRVVHLYRNGKDQDAEQAPGLQGLE
PKSCDKTHTCPPCPELKTPLGDTTHTSRGQYSREDNNCTHFPVGQSHMLLELRTAFS
QVKTFFQTKDQLDNILLTDSLMQDFKGYLGCQALSEMIQFYLVEVMPQAEKHGPEIKE
HLNSLGEKLKTLRMRLRRCHRFLPCENKSKAVEQVKSDFNKLQDQGVYKAMNEFDIFI
NCIEAYMMIKMKS
SEQ ID NO: 26
Amino acid sequence of VB5066. Mouse immunoglobulin heavy chain signal sequence
“lg VH signal seq” (1-19), Mouse single chain variable fragment “scFv” anti-DEC205
(20-265), Hinge h1 hIgG3 (266-277), Hinge h4 hIgG3 (278-292), linker (293-297), MOG
amino acids 27-63 (298-334), Upper hinge region hIgG1 (335-339), Middle hinge
region hIgG1 (340-354), Murine TGFβ1 mature sequence (355-466).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLSPGKNATGMEVGWYRSPFSRVVHLYRNGKDQDAEQAPGLQGLE
PKSCDKTHTCPPCPALDTNYCFSSTEKNCCVRQLYIDFRKDLGWKWIHEPKGYHANF
CLGPCPYIWSLDTQYSKVLALYNQHNPGASASPCCVPQALEPLPIVYYVGRKPKVEQL
SNMIVRSCKCS
SEQ ID NO: 27
Amino acid sequence of VB5043. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), MOG
amino acids 27-63 (298-334), Upper hinge region hIgG1 (335-339), Middle hinge
region hIgG1 (340-354), Hinge h1 hIgG3 (355-366), Murine TGFβ1 mature sequence
(355-478).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLSPGKNATGMEVGWYRSPFSRVVHLYRNGKDQDAEQAPGLQGLE
PKSCDKTHTCPPCPELKTPLGDTTHTALDTNYCFSSTEKNCCVRQLYIDFRKDLGWK
WIHEPKGYHANFCLGPCPYIWSLDTQYSKVLALYNQHNPGASASPCCVPQALEPLPIV
YYVGRKPKVEQLSNMIVRSCKCS
SEQ ID NO: 28
Amino acid sequence of VB5067. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), MOG
amino acids 27-63 (298-334), Upper hinge region hIgG1 (335-339), Middle hinge
region hIgG1 (340-354), Murine CTLA-4 (355-480).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLSPGKNATGMEVGWYRSPFSRVVHLYRNGKDQDAEQAPGLQGLE
PKSCDKTHTCPPCPEAIQVTQPSVVLASSHGVASFPCEYSPSHNTDEVRVTVLRQTN
DQMTEVCATTFTEKNTVGFLDYPFCSGTFNESRVNLTIQGLRAVDTGLYLCKVELMYP
PPYFVGMGNGTQIYVIDPEPCPDSD
SEQ ID NO: 29
Amino acid sequence of VB5072. Murine MARCO ligand SCGB3A2signal sequence
(1-21), Murine MARCO ligand SCGB3A2 (22-91), Hinge h1 hIgG3 (92-103), Hinge h4
hIgG3 (104-118), linker (119-123), MOG amino acids 27-63 (124-160), Upper hinge
region hIgG1 (161-165), Middle hinge region hIgG1 166-180), Murine IL-10 (181-340)
MKLVSIFLLVTIGICGYSATALLINRLPVVDKLPVPLDDIIPSFDPLKMLLKTLGISVEHLVT
GLKKCVDELGPEASEAVKKLLEALSHLVELKTPLGDTTHTEPKSCDTPPPCPRCPGLG
GLSPGKNATGMEVGWYRSPFSRVVHLYRNGKDQDAEQAPGLQGLEPKSCDKTHTC
PPCPSRGQYSREDNNCTHFPVGQSHMLLELRTAFSQVKTFFQTKDQLDNILLTDSLM
QDFKGYLGCQALSEMIQFYLVEVMPQAEKHGPEIKEHLNSLGEKLKTLRMRLRRCHR
FLPCENKSKAVEQVKSDFNKLQDQGVYKAMNEFDIFINCIEAYMMIKMKS
SEQ ID NO: 30
Amino acid sequence of VB5073. Murine MARCO ligand SCGB3A2signal sequence
(1-21), Murine MARCO ligand SCGB3A2 (22-91), Hinge h1 hIgG3 (92-103), Hinge h4
hIgG3 (104-118), linker (119-123), MOG amino acids 27-63 (124-160), Upper hinge
region hIgG1 (161-165), Middle hinge region hIgG1 166-180), Hinge h1 hIgG3 (181-
192), Murine IL-10 (193-352)
MKLVSIFLLVTIGICGYSATALLINRLPVVDKLPVPLDDIIPSFDPLKMLLKTLGISVEHLVT
GLKKCVDELGPEASEAVKKLLEALSHLVELKTPLGDTTHTEPKSCDTPPPCPRCPGLG
GLSPGKNATGMEVGWYRSPFSRVVHLYRNGKDQDAEQAPGLQGLEPKSCDKTHTC
PPCPELKTPLGDTTHTSRGQYSREDNNCTHFPVGQSHMLLELRTAFSQVKTFFQTKD
QLDNILLTDSLMQDFKGYLGCQALSEMIQFYLVEVMPQAEKHGPEIKEHLNSLGEKLK
TLRMRLRRCHRFLPCENKSKAVEQVKSDFNKLQDQGVYKAMNEFDIFINCIEAYMMIK
MKS
SEQ ID NO: 31
Amino acid sequence of VB5074. Murine VISTA ligand VSIG-3 signal sequence (1-
22), Murine VISTA ligand VSIG-3 extracellular domain (23-240), Hinge h1 hIgG3 (241-
252), Hinge h4 hIgG3 (253-267), linker (268-272), MOG amino acids 27-63 (273-309),
Upper hinge region hIgG1 (310-314), Middle hinge region hIgG1 (315-329), Murine IL-
10 (330-489).
MTRRRSAPASWLLVSLLGVATSLEVSESPGSVQVARGQTAVLPCAFSTSAALLNLNVI
WMVIPLSNANQPEQVILYQGGQMFDGALRFHGRVGFTGTMPATNVSIFINNTQLSDT
GTYQCLVNNLPDRGGRNIGVTGLTVLVPPSAPQCQIQGSQDLGSDVILLCSSEEGIPR
PTYLWEKLDNTLKLPPTATQDQVQGTVTIRNISALSSGLYQCVASNAIGTSTCLLDLQV
ISPQPRSVELKTPLGDTTHTEPKSCDTPPPCPRCPGLGGLSPGKNATGMEVGWYRS
PFSRVVHLYRNGKDQDAEQAPGLQGLEPKSCDKTHTCPPCPSRGQYSREDNNCTHF
PVGQSHMLLELRTAFSQVKTFFQTKDQLDNILLTDSLMQDFKGYLGCQALSEMIQFYL
VEVMPQAEKHGPEIKEHLNSLGEKLKTLRMRLRRCHRFLPCENKSKAVEQVKSDFNK
LQDQGVYKAMNEFDIFINCIEAYMMIKMKS
SEQ ID NO: 32
Amino acid sequence of VB5075. Murine VISTA ligand VSIG-3 signal sequence (1-
22), Murine VISTA ligand VSIG-3 extracellular domain (23-240), Hinge h1 hIgG3 (241-
252), Hinge h4 hIgG3 (253-267), linker (268-272), MOG amino acids 27-63 (273-309),
Upper hinge region hIgG1 (310-314), Middle hinge region hIgG1 (315-329), Hinge h1
hIgG3 (330-341), Murine IL-10 (342-501).
MTRRRSAPASWLLVSLLGVATSLEVSESPGSVQVARGQTAVLPCAFSTSAALLNLNVI
WMVIPLSNANQPEQVILYQGGQMFDGALRFHGRVGFTGTMPATNVSIFINNTQLSDT
GTYQCLVNNLPDRGGRNIGVTGLTVLVPPSAPQCQIQGSQDLGSDVILLCSSEEGIPR
PTYLWEKLDNTLKLPPTATQDQVQGTVTIRNISALSSGLYQCVASNAIGTSTCLLDLQV
ISPQPRSVELKTPLGDTTHTEPKSCDTPPPCPRCPGLGGLSPGKNATGMEVGWYRS
PFSRVVHLYRNGKDQDAEQAPGLQGLEPKSCDKTHTCPPCPELKTPLGDTTHTSRG
QYSREDNNCTHFPVGQSHMLLELRTAFSQVKTFFQTKDQLDNILLTDSLMQDFKGYL
GCQALSEMIQFYLVEVMPQAEKHGPEIKEHLNSLGEKLKTLRMRLRRCHRFLPCENK
SKAVEQVKSDFNKLQDQGVYKAMNEFDIFINCIEAYMMIKMKS
SEQ ID NO: 33
Amino acid sequence of VB5052. Human CCL3L1 signal sequence “Mip1a” (1-23),
Human CCL3L1 “hMip1a” (24-93), Hinge h1 hIgG3 (94-105), Hinge h4 hIgG3 (106-
120), linker (121-130), hCH3 IgG3 (131-237), linker (238-242), MOG amino acids 27-
63 (243-279).
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLSPGKNATGMEVGWYRSPFSRVVHLYRNGKDQDAEQAP
SEQ ID NO: 34
Amino acid sequence of VB5002b. Human CCL3L1 signal sequence “Mip1a” (1-23),
Human CCL3L1 “hMip1a” (24-93), Hinge h1 hIgG3 (94-105), Hinge h4 hIgG3 (106-
120), linker (121-130), hCH3 lgG3 (131-237), linker (238-242), MOG amino acids 27-
63 (243-279). Parts of MOG (27-63) sequence was obtained from the article Krienke et
al. 2021.
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLSPGKNATGMEVGWYRSPFSRVVHLYRNGKDQDAEQAP
SEQ ID NO: 35
Amino acid sequence of VB5051. Mouse immunoglobulin heavy chain signal sequence
“Ig VH signal seq” (1-19), MOG amino acids 27-63 (20-56).
MNFGLRLIFLVLTLKGVQCSPGKNATGMEVGWYRSPFSRVVHLYRNGKDQDAEQAP
SEQ ID NO: 36
Amino acid sequence of VB5001b. Mouse immunoglobulin heavy chain signal
sequence “Ig VH signal seq” (1-19), MOG amino acids 27-63 (20-56). Parts of MOG
(27-63) sequence were obtained from the article Krienke et al. 2021.
MNFGLRLIFLVLTLKGVQCSPGKNATGMEVGWYRSPFSRVVHLYRNGKDQDAEAQP
SEQ ID NO: 37
Amino acid sequence of VB5077. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), Met
e 1”241-260”, “210-230”, “136-155”, “76-95”, ”46-65”, “16-35” (293-468), Upper hinge
region hIgG1 (469-473), Middle hinge region hIgG1 (474-488), Murine IL-10 (489-648).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLKEVDRLEDELVNEKEKYKSIGGGGSGGGGSAYKEQIKTLTNKLKA
AEARAEGGGGSGGGGSNQLKEARFLAEEADRKYDEVGGGGSGGGGSAALNRRIQL
LEEDLERSEERGGGGSGGGGSDLDQVQESLLKANNQLVEKDGGGGSGGGGSEQQ
NKEANNRAEKSEEEVHNGLQGLEPKSCDKTHTCPPCPSRGQYSREDNNCTHFPVG
QSHMLLELRTAFSQVKTFFQTKDQLDNILLTDSLMQDFKGYLGCQALSEMIQFYLVEV
MPQAEKHGPEIKEHLNSLGEKLKTLRMRLRRCHRFLPCENKSKAVEQVKSDFNKLQD
QGVYKAMNEFDIFINCIEAYMMIKMKS
SEQ ID NO: 38
Amino acid sequence of VB5078. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), Met
e 1”241-260”, “210-230”, “136-155”, “76-95”, ”46-65”, “16-35” (293-468), Upper hinge
region hIgG1 (469-473), Middle hinge region hIgG1 (474-488), Hinge h1 hIgG3 (489-
500), Murine IL-10 (501-660).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLKEVDRLEDELVNEKEKYKSIGGGGSGGGGSAYKEQIKTLTNKLKA
AEARAEGGGGSGGGGSNQLKEARFLAEEADRKYDEVGGGGSGGGGSAALNRRIQL
LEEDLERSEERGGGGSGGGGSDLDQVQESLLKANNQLVEKDGGGGGGGGSEQQ
NKEANNRAEKSEEEVHNGLQGLEPKSCDKTHTCPPCPELKTPLGDTTHTSRGQYSR
EDNNCTHFPVGQSHMLLELRTAFSQVKTFFQTKDQLDNILLTDSLMQDFKGYLGCQA
LSEMIQFYLVEVMPQAEKHGPEIKEHLNSLGEKLKTLRMRLRRCHRFLPCENKSKAVE
QVKSDFNKLQDQGVYKAMNEFDIFINCIEAYMMIKMKS
SEQ ID NO: 41
Amino acid sequence of VB5041. Mouse immunoglobulin heavy chain signal sequence
“Ig VH signal seq” (1-19), Mouse single chain variable fragment “scFv” anti-DEC205
(20-265), Hinge h1 hIgG3 (266-277), Hinge h4 hIgG3 (278-292), linker (293-297), MOG
amino acids 35-55 (298-318), Upper hinge region hIgG1 (319-323), Middle hinge
region hIgG1 (323-338), Hinge h1 hIgG3 (339-350), Murine IL-10 (315-510).
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGLGGLMEVGWYRSPFSRVVHLYRNGKGLQGLEPKSCDKTHTCPPCPELK
TPLGDTTHTSRGQYSREDNNCTHFPVGQSHMLLELRTAFSQVKTFFQTKDQLDNILLT
DSLMQDFKGYLGCQALSEMIQFYLVEVMPQAEKHGPEIKEHLNSLGEKLKTLRMRLR
RCHRFLPCENKSKAVEQVKSDFNKLQDQGVYKAMNEFDIFINCIEAYMMIKMKS

### EMBODIMENTS

1. A tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii), such as a dimeric protein consisting of two polypeptides as defined in ii).

2. A tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein consisting of multiple polypeptides as defined in ii).

3. A tolerance-inducing construct comprising:
i) a polynucleotide comprising a nucleotide sequence encoding a polypeptide, the polypeptide comprising, in the specified order:
   a. a first targeting unit, a first joint region;
   b. an antigenic unit;
   c. a second joint region; and
   d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a dimeric protein consisting of two polypeptides as defined in ii) . . .

4. The tolerance-inducing construct of any one of the preceding embodiments, wherein the multimeric protein, such as the dimeric protein, consists of multiple polypeptides, such as two polypeptides, that are linked to each other via their joint regions, preferably via their respective first joint regions and via their respective second joint regions.

5. The tolerance-inducing construct of any one of the preceding embodiments, wherein the multimeric protein consists of multiple polypeptides that are linked to each other via their joint regions, preferably via their respective first joint regions and via their respective second joint regions.

6. The tolerance-inducing construct of any one of the preceding embodiments, wherein the dimeric protein consists of two polypeptides that are linked to each other via their joint regions, preferably via their respective first joint regions and via their respective second joint regions.

7. The tolerance-inducing construct of any one of the preceding embodiments, wherein the first- and second joint regions comprise a flexible unit and a binding unit.

8. The tolerance-inducing construct of any of the preceding embodiments, wherein the first- and/or second joint regions comprise a binding unit which is a non-covalent binding unit.

9. The tolerance-inducing construct of any of the preceding embodiments, wherein the non-covalent binding unit is a trimerization unit.

10. The tolerance-inducing construct of any of embodiments 1-9, wherein the trimerization unit is a collagen-derived trimerization unit.

11. The tolerance-inducing construct of embodiment 10, wherein the collagen-derived trimerization unit is a human collagen derived XVIII trimerization domain.

12. The tolerance-inducing construct of embodiment 10, wherein the collagen-derived trimerization unit is a human collagen XV trimerization domain.

13. The tolerance-inducing construct of any of embodiments 1-8, wherein the non-covalent binding unit is a tetramerization unit.

14. The tolerance-inducing construct of embodiment 13, wherein the tetramerization domain is a domain derived from p53.

15. The tolerance-inducing construct of any of embodiments 1-8, wherein the non-covalent binding unit is a dimerization unit.

16. The tolerance-inducing construct of embodiment 15, wherein the dimerization unit comprises a hinge region and an immunoglobulin domain.

17. The tolerance-inducing construct of embodiment 16, wherein the dimerization unit is an immunoglobulin constant domain.

18. The tolerance-inducing construct of embodiment 15, wherein the dimerization unit comprises the dHLX protein.

19. The tolerance-inducing construct of any of the preceding embodiments, wherein the first- and/or second joint regions comprise a binding unit which is a covalent binding unit.

20. The tolerance-inducing construct of any of the preceding embodiments, wherein the first- and/or second joint regions comprise or consist of a naturally occurring sequence.

21. The tolerance-inducing construct of any of embodiments 1-19, wherein the first- and/or second joint regions comprise or consist of an artificial sequence.

22. The tolerance-inducing construct of any of embodiments 19-21, wherein the first and second joint regions comprise a covalent binding unit which comprises one or more cysteine residues.

23. The tolerance-inducing construct of embodiment 22, wherein the covalent binding unit comprises at least 2 cysteine residues.

24. The tolerance-inducing construct of embodiment 22-23, wherein the covalent binding unit comprises at least 2 cysteine residues, such as at least 3, 4, 5, 6, 7, 8, 9, 10, 11, 12 or 13 cysteine residues.

25. The tolerance-inducing construct of embodiments 19 to 22, wherein the covalent binding unit comprises a cysteine rich sequence.

26. The tolerance-inducing construct of any of embodiments 19-25, wherein the covalent binding unit of the first joint region comprises a different number of cysteine residues than the covalent binding unit of the second joint region.

27. The tolerance-inducing construct of any of embodiments 22-26, wherein the cysteine residues comprised in the covalent binding unit of the first joint region are positioned differently than the cysteine residues comprised in the covalent binding unit of the second joint region.

28. The tolerance-inducing construct of embodiment 22-27, wherein the number of amino acid residues between the cysteine residues of the covalent binding unit of the first joint region is different than that of the second joint region.

29. The tolerance-inducing construct of any of embodiments 22-28, wherein the number of cysteine residues is based on the length of the antigenic unit.

30. The tolerance-inducing construct of any of embodiments 19-29, wherein at least one of the covalent binding units is derived from an immunoglobulin.

31. The tolerance-inducing construct of embodiment 30, wherein the covalent binding unit is a hinge region derived from an immunoglobulin, such as exon h4 of IgG3 or the middle hinge of IgG1.

32. The tolerance-inducing construct of embodiment 30, wherein the hinge region is Ig derived, such as derived from IgG, e.g. IgG1, IgG2 or IgG3.

33. The tolerance-inducing construct of embodiment 30, wherein the hinge region is derived from IgM.

34. The tolerance-inducing construct of embodiment 30, wherein the hinge region comprises or consists of the nucleotide sequence with SEQ ID NO: 157 or an amino acid sequence encoded by said nucleotide sequence.

35. The tolerance-inducing construct of embodiment 30, wherein the covalent binding unit comprises or consists of an amino acid sequence having at least 40% sequence identity to the amino acid sequence 13-27 of SEQ ID NO: 1, such as at least 50%, at least 60%, at least 70%, at least 80% or at least 90% sequence identity.

36. The tolerance-inducing construct of embodiment 30, wherein covalent binding unit comprises or consists of the amino acid sequence 13-27 of SEQ ID NO: 1, wherein any one of the amino acids of the flexible unit has been substituted, deleted, or inserted for another amino acid, with the proviso that no more than 6 amino acids have been so substituted, deleted, or inserted, such as no more than 5 amino acids, such as no more than 4 amino acids, such as no more than 3 amino acids, such as no more than 2 amino acids or no more than 1 amino acid.

37. The tolerance-inducing construct of embodiment 30, wherein the covalent binding unit consists of amino acid sequence 13-23 27 of SEQ ID NO: 1.

38. The tolerance-inducing construct of embodiment 31, wherein the covalent binding unit is hinge exon h4 of IgG3.

39. The tolerance-inducing construct of embodiments 30, wherein the covalent binding unit comprises the sequence EPKSCDTPPPCPRCP (SEQ ID NO: 156; corresponding to amino acids 13-27 of SEQ ID NO: 1).

40. The tolerance-inducing construct of any of embodiments 1-30, wherein the covalent binding unit comprises or consists of an amino acid sequence having at least 40% sequence identity to the amino acid sequence 5-15 of SEQ ID NO: 2, provided that the cysteine residues are retained in their number and position, such as at least 50%, at least 60%, at least 70%, at least 80% or at least 90% sequence identity.

41. The tolerance-inducing construct of embodiments 1-30, wherein the covalent binding unit comprises or consists of the amino acid sequence 5-15 of SEQ ID NO: 2, wherein any one of the amino acids of the flexible unit has been substituted, deleted, or inserted for another amino acid, with the proviso that no more than 5 amino acids have been so substituted, deleted, or inserted, such as no more than 4 amino acids, such as no more than 3 amino acids, such as no more than 2 amino acids or no more than 1 amino acid.

42. The tolerance-inducing construct of embodiments 41, wherein the covalent binding unit consists of or comprises the amino acid sequence 5-15 of SEQ ID NO: 2.

43. The tolerance-inducing construct of embodiments 30, wherein the covalent binding unit is the middle hinge region of IgG1.

44. The tolerance-inducing construct of any of embodiments 19-43, wherein the covalent binding unit is a non-immunogenic sequence.

45. The tolerance-inducing construct of any of embodiments 19-44, wherein the covalent binding unit is a naturally occurring peptide sequence.

46. The tolerance-inducing construct of any one of embodiments 19-45, wherein the covalent binding unit consists of from 2 to 100 amino acids, such as 3 to 70 amino acids, such as 4 to 50 amino acids or 5 to 30 amino acids.

47. The tolerance-inducing construct of any one of embodiments 19-46, wherein the covalent binding unit consists of 10, 11, 12, 13, 14, 15, 16, 17, 18, 19 or 20 amino acids.

48. The tolerance-inducing construct of any of embodiments 19-47, wherein at least one of the covalent binding units is an artificial sequence.

49. The tolerance-inducing construct of any of embodiments 8-48, wherein the first and second joint regions comprise a binding unit which is a non-covalent binding unit.

50. The tolerance-inducing construct of any of embodiments 8-49, wherein the non-covalent binding unit contributes to multimerization through non-covalent interactions, such as hydrophobic interactions.

51. The tolerance-inducing construct of any of embodiments 8-50, wherein the non-covalent binding unit contributes to dimerization through non-covalent interactions, such as hydrophobic interactions.

52. The tolerance-inducing construct of any of embodiments 8-51, wherein the non-covalent binding unit has the ability to form multimeric proteins via non-covalent interactions.

53. The tolerance-inducing construct of any of embodiments 8-52, wherein the non-covalent binding unit has the ability to form dimers via non-covalent interactions.

54. The tolerance-inducing construct of any of embodiments 8-53, wherein at least one of the non-covalent binding units is a naturally occurring sequence.

55. The tolerance-inducing construct of any of embodiments 8-54, wherein at least one of the non-covalent binding units is an artificial sequence.

56. The tolerance-inducing construct of any of embodiments 8-55, wherein the non-covalent binding unit is or comprises an immunoglobulin.

57. The tolerance-inducing construct of any of embodiments 8-56, wherein the non-covalent binding unit consists of or comprises an immunoglobulin domain, such as an immunoglobulin constant domain (C domain), such as a carboxyterminal constant domain (i.e. a CH3 domain), a CH1 domain or a CH2 domain, or a sequence that is substantially identical to the C domain or a variant thereof.

58. The tolerance-inducing construct of any one of embodiments 8-57, wherein the non-covalent binding unit comprises or consists of a CH3 domain derived from IgG, such as derived from IgG3 or IgG1, preferably derived from IgG1.

59. The tolerance-inducing construct of any one of embodiment 8-58, wherein the non-covalent binding unit comprises or consists of a CH3 domain derived from IgG3.

60. The tolerance-inducing construct of any one of embodiment 8-59, wherein the non-covalent binding unit comprises or consists of a carboxyterminal C domain derived from IgG3 with an amino acid sequence having at least 80% sequence identity to the amino acid sequence of SEQ ID NO: 3.

61. The tolerance-inducing construct of any one of embodiment 8-60, wherein the non-covalent binding unit comprises or consists of a carboxyterminal C domain derived from IgG3 with an amino acid sequence having at least 85% sequence identity to the amino acid sequence of SEQ ID NO: 3, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

62. The tolerance-inducing construct of any one of embodiment 8-61, wherein the non-covalent binding unit comprises or consists of a carboxyterminal C domain derived from IgG3 with the amino acid sequence of SEQ ID NO: 3, wherein any one of the amino acids of the flexible unit has been substituted, deleted, or inserted for another amino acid, with the proviso that no more than 21 amino acids have been so substituted, deleted, or inserted, such as no more than 20 amino acids, such as no more than 19 amino acids, such as no more than 18 amino acids, such as no more than 17 amino acids, such as no more than 16 amino acids, such as no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids, such as no more than 6 amino acids, such as no more than 5 amino acids, such as no more than 4 amino acids, such as no more than 3 amino acids, such as no more than 2 amino acids, such as no more than 1 amino acid.

63. The tolerance-inducing construct of any one of 8-62, wherein the non-covalent binding unit comprises or consists of a CH3 domain from IgG1.

64. The tolerance-inducing construct of any one of 8-63, wherein the non-covalent binding unit comprises or consists of CH3 domain derived from IgG1 with an amino acid sequence having at least 80% sequence identity to the amino acid sequence of SEQ ID NO: 4.

65. The tolerance-inducing construct of any one of 8-64, wherein the non-covalent binding unit comprises or consists of a CH3 domain from IgG1 with an amino acid sequence having at least 85% sequence identity to the amino acid sequence according to SEQ ID NO: 4, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

66. The tolerance-inducing construct of any one of 8-65, wherein the non-covalent binding unit comprises or consists of a CH3 domain derived from IgG1 with the amino acid sequence of SEQ ID NO: 3, wherein any one of the amino acids of the flexible unit has been substituted, deleted, or inserted for another amino acid, with the proviso that no more than 21 amino acids have been so substituted, deleted, or inserted, such as no more than 20 amino acids, such as no more than 19 amino acids, such as no more than 18 amino acids, such as no more than 17 amino acids, such as no more than 16 amino acids, such as no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids, such as no more than 6 amino acids, such as no more than 5 amino acids, such as no more than 4 amino acids, such as no more than 3 amino acids, such as no more than 2 amino acids, such as no more than 1 amino acid.

67. The tolerance-inducing construct of any one of 8-66, wherein the non-covalent binding unit comprises or consists of a leucine zipper motif.

68. The tolerance-inducing construct of embodiment 67, wherein the leucine zipper motif is derived from the bZIP class of eukaryotic transcription factors.

69. The tolerance-inducing construct of any one of 8-67, wherein the non-covalent binding unit comprises or consists of a Jun/Fos-based leucine zipper.

70. The tolerance-inducing construct of any one of 8-67, wherein the leucine zipper motif comprises or consists of the amino acid sequence of SEQ ID NO: 5.

71. The tolerance-inducing construct of any one of 8-67, wherein the non-covalent binding unit comprises or consists of an amino acid sequence having at least 80% sequence identity to the amino acid sequence of SEQ ID NO: 5, such as at least 81% or at least 81%, 83%, 84%, 85%, 86%, 87%, 88%, 89%, 90%, 91%, 92%, 93%, 94%, 95%, 96%, 97%, 98% or at least 99% sequence identity.

72. The tolerance-inducing construct of any one of 8-67, wherein the non-covalent binding unit comprises or consists of the amino acid sequence of SEQ ID NO: 5, wherein any one of the amino acids of the flexible unit has been substituted, deleted, or inserted for another amino acid, with the proviso that no more than 12, such as no more than 11, such as no more than 10, such as no more than 9, such as no more than 8, such as no more than 7, such as no more than 6, such as no more than 5, such as no more than 4 amino acids, such as no more than 3 amino acids, such as no more than 2 amino acids, such as no more than 1 amino acid.

73. The tolerance-inducing construct of any one of 8-72, wherein the non-covalent binding unit joins multiple polypeptides, such as two, three, four or more polypeptides, into a multimeric protein, such as a dimeric protein, a trimeric protein or a tetrameric protein.

74. The tolerance-inducing construct of any one of 8-73, wherein the non-covalent binding unit is or comprises a trimerization unit, such as a collagen-derived trimerization unit, such as a human collagen-derived trimerization domain, such as human collagen derived XVIII trimerization domain or human collagen XV trimerization domain.

75. The tolerance-inducing construct of any one of 8-74, wherein the non-covalent binding unit is a trimerization unit that comprises or consists of the nucleotide sequence with SEQ ID NO: 158, or an amino acid sequence encoded by said nucleotide sequence.

76. The tolerance-inducing construct of any one of 8-75, wherein the trimerization unit comprises or consists of the C-terminal domain of T4 fibritin.

77. The tolerance-inducing construct of any one of 8-76, wherein the non-covalent binding unit is a trimerization unit that comprises or consists of the amino acid sequence with SEQ ID NO: 159, or a nucleotide sequence encoding said amino acid sequence.

78. The tolerance-inducing construct of any one of 8-77, wherein the non-covalent binding unit comprises or consists of a tetramerization unit, such as a domain derived from p53.

79. The tolerance-inducing construct of any one of 8-78, wherein the non-covalent binding unit is a tetramerization unit that comprises or consists of the nucleotide sequence with SEQ ID NO: 160, or an amino acid sequence encoded by said nucleotide sequence.

80. The tolerance-inducing construct of any of the preceding embodiments, wherein the first- and/or second joint regions comprise or consist of a naturally occurring sequence.

81. The tolerance-inducing construct of any of the preceding embodiments, wherein the first- and/or second joint regions comprise or consist of an artificial sequence.

82. The tolerance-inducing construct of any of the preceding embodiments, wherein the number of cysteine residues in the first- and/or second joint regions is based on the length of the antigenic unit.

83. The tolerance-inducing construct of any of the preceding embodiments, wherein the joint regions comprise a binding unit which comprises a covalent binding unit and a non-covalent binding unit.

84. The tolerance-inducing construct of any of the preceding embodiments, wherein the joint regions are non-immunogenic 85. The tolerance-inducing construct of any of the preceding embodiments 7 to 84, wherein the flexible unit is between the targeting unit and the binding unit.

86. The tolerance-inducing construct of any of the preceding embodiments 7 to 85, wherein the flexible unit is a non-immunogenic sequence.

87. The tolerance-inducing construct of any of the preceding embodiments 7 to 86, wherein at least one of the flexible units is a naturally occurring peptide sequence.

88. The tolerance-inducing construct of any of the preceding embodiments 7 to 87, wherein the flexible unit is derived from an immunoglobulin.

89. The tolerance-inducing construct of any of the preceding embodiments 7 to 88, wherein the flexible unit is a hinge region derived from an immunoglobulin, such as exon h1 of IgG3 or the lower hinge of IgG1.

90. The tolerance-inducing construct of any of the preceding embodiments 7 to 89, wherein the flexible unit comprises or consists of hinge exon h1 of IgG3.

91. The tolerance-inducing construct of any of the preceding embodiments 7 to 90, wherein the flexible unit comprises or consists of the lower hinge region of IgG1.

92. The tolerance-inducing construct of any of the preceding embodiments 7 to 91, wherein the flexible unit comprises or consists of the amino acid sequence 1-12 of SEQ ID NO: 1.

93. The tolerance-inducing construct of any of the preceding embodiments 7 to 92, wherein the flexible unit comprises or consists of an amino acid sequence having at least 50% sequence identity to the an amino acid sequence 16-23 of SEQ ID NO: 2, such as 60% or such as 70% or such as 80% or such as 90% sequence identity.

94. The tolerance-inducing construct of any of the preceding embodiments 7 to 93, wherein any one of the amino acids of the flexible unit has been substituted, deleted, or inserted for another amino acid, with the proviso that no more than 5 amino acids have been so substituted, deleted, or inserted, such as no more than 4 amino acids, such as no more than 3 amino acids, such as no more than 2 amino acids or no more than 1 amino acid.

95. The tolerance-inducing construct of any of the preceding embodiments 7 to 94, wherein the flexible unit is derived from an immunoglobulin, such as a hinge region of an immunoglobulin, such as hinge region of an immunoglobulin not comprising cysteine residues.

96. The tolerance-inducing construct of any of the preceding embodiments 7 to 95, wherein at least one of the flexible units is an artificial sequence.

97. The tolerance-inducing construct of any of embodiments 7-83 and 96, wherein the flexible unit is a serine and/or glycine rich linker.

98. The tolerance-inducing construct of any of embodiments 7-84 and 97-98, wherein the flexible unit is a glycine-serine linker, such as GGGGSGGGGS (SEQ ID NO: 80).

99. The tolerance-inducing construct of any of the preceding embodiments 7 to 99, wherein the flexible unit is not a target of proteases.

100. The tolerance-inducing construct of any of embodiments 7 to 99, wherein the flexible unit consists of up to 20 amino acids, such as at up to 15 amino acids, such as 12 amino acids or 10 amino acids.

101. The tolerance-inducing construct of any of embodiments 7 to 100, wherein the flexible unit comprised in the second joint region consists of from 5 to 60 amino acids, such as from 7 to 55 amino acids or 8 to 50 amino acids or 9 to 45 amino acids or 10 to 40 amino acids or 11 to 35 amino acids or 12 to 30 amino acids or 13 to 20 amino acids.

102. The tolerance-inducing construct of any of embodiments 7 to 101, wherein the flexible unit comprises small, non-polar amino acids, such as glycine, alanine or leucine, or polar amino acids, such as serine or threonine.

103. The tolerance-inducing construct of any of preceding embodiments, wherein the joint regions are non-immunogenic.

104. The tolerance-inducing construct of any of the preceding embodiments, wherein at least one of the first- or the second targeting units comprises a moiety that interacts with surface molecules on the antigen-presenting cells, preferably wherein both the first and the second targeting units comprises a moiety that interacts with surface molecules on the antigen-presenting cells.

105. The tolerance-inducing construct of embodiment 104, wherein the surface molecule is selected from the group consisting of TGFβ receptor (TGFβR1, TGFβR2, or TGFβR3), IL10R, such as IL-10RA and IL10-RB, IL2R, IL4R, IL6R, IL11R and IL13R, IL27R, IL35R, IL37R, CCR7, CD11b, CD11c, CD103, CD14, CD36, CD205, CD109, VISTA, MARCO, MHCII, MHCII, CD83, SIGLEC, MGL, CD80, CD86, Clec9A, Clec12A, Clec12B, DCIR2, Langerin, MR, DC-Sign, Treml4, Dectin-1, PDL1, PDL2, HVEM, arylhydrocarbon receptor and vitamin D receptor.

106. The tolerance-inducing construct of embodiment 105, wherein the targeting unit comprises a moiety which is a natural ligand, an antibody or part thereof, e.g. a scFv, or a synthetic ligand.

107. The tolerance-inducing construct of embodiment 106, wherein the natural ligand is selected from the group consisting of TGFβ, IL-10, IL1RA, IL2, IL4, IL6, IL11, IL13, IL27, IL35, IL37, CCL19, CCL21, ICAM-1 (Intercellular Adhesion Molecule 1 also known as CD54), keratin, VSIG-3, SCGB3A2, CTLA-4, preferably the extracellular domain of CTLA-4, PD-1, preferably the extracellular domain of PD-1 and BTLA, preferably the extracellular domain of BTLA.

108. The tolerance-inducing construct of any one of the previous embodiments, wherein the targeting unit comprises or consists of IL-10 or TGFβ, preferably human IL-10 or human TGFβ.

109. The tolerance-inducing construct of any one of the previous embodiments, wherein the targeting unit comprises or consists of an amino acid sequence having at least 80% sequence identity to that of human TGFβ.

110. The tolerance-inducing construct of any one of the previous embodiments, wherein the targeting unit comprises or consists of an amino acid sequence having at least 85% sequence identity to the amino acid sequence of human TGFβ, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

111. The tolerance-inducing construct of any one of the previous embodiments, wherein the targeting unit comprises or consists of an amino acid sequence of human TGFβ, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

112. The tolerance-inducing construct of any one of the previous embodiments, wherein the targeting unit comprises or consists of an amino acid sequence having at least 80% sequence identity to that of human IL-10.

113. The tolerance-inducing construct of any one of the previous embodiments, wherein the targeting unit comprises or consists of an amino acid sequence having at least 85% sequence identity to the amino acid sequence of human IL-10, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

114. The tolerance-inducing construct of any one of the previous embodiments, wherein the targeting unit comprises or consists of an amino acid sequence of human IL-10, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

115. The tolerance-inducing construct of any one of the previous embodiments, wherein the targeting unit comprises or consists of an amino acid sequence of human IL-10, or a nucleotide sequence encoding human IL-10.

116. The tolerance-inducing construct of any one of the previous embodiments, wherein the targeting unit is or comprises SCGB3A2or VSIG-3, preferably human VSIG-3 or human SCGB3A2.

117. The tolerance-inducing construct of any one of the previous embodiments, wherein the targeting unit comprises or consists of an amino acid sequence having at least 80% sequence identity to that of human SCGB3A2.

118. The tolerance-inducing construct of any one of the previous embodiments, wherein the targeting unit comprises or consists of an amino acid sequence having at least 85% sequence identity to the amino acid sequence of human SCGB3A2, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

119. The tolerance-inducing construct of any one of the previous embodiments, wherein the targeting unit comprises or consists of an amino acid sequence of human SCGB3A2, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

120. The tolerance-inducing construct of any one of the previous embodiments, wherein the targeting unit comprises or consists of an amino acid sequence of human SCGB3A2, or a nucleotide sequence encoding human SCGB3A2.

121. The tolerance-inducing construct of any one of the previous embodiments, wherein the targeting unit comprises or consists of an amino acid sequence having at least 80% sequence identity to that of human VSIG-3.

122. The tolerance-inducing construct of any one of the previous embodiments, wherein the targeting unit comprises or consists of an amino acid sequence having at least 85% sequence identity to the amino acid sequence of human VSIG-3, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

123. The tolerance-inducing construct of any one of the previous embodiments, wherein the targeting unit comprises or consists of an amino acid sequence of human VSIG-3, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

124. The tolerance-inducing construct of any one of the previous embodiments, wherein the targeting unit comprises or consists of an amino acid sequence of human VSIG-3, or a nucleotide sequence encoding human VSIG-3.

125. The tolerance-inducing construct of any one of the previous embodiments, wherein the targeting unit comprises or consists of an antibody or part thereof, e.g. a scFv, with specificity for CD205.

126. The tolerance-inducing construct of any of the preceding embodiments, wherein the first- and the second targeting units are identical.

127. The tolerance-inducing construct of any of embodiments 1 to 125, wherein the first- and the second targeting units are different.

128. The tolerance-inducing construct of any of embodiments 104 to 127, wherein the surface molecules are present on the same cell.

129. The tolerance-inducing construct of any of embodiments 104 to 128, wherein binding of the first- or the second targeting unit causes internalization of the construct.

130. The tolerance-inducing construct of any of the preceding embodiments, wherein the antigenic unit is located between the first and the second joint region.

131. The tolerance-inducing construct of any of the preceding embodiments, wherein the antigenic unit comprises one or more T cell epitopes of a self-antigen, such as one T cell epitope of a self-antigen or more than one T cell epitope of a self-antigen, such as multiple T cell epitopes of a self-antigen.

132. The tolerance-inducing construct of embodiment 131, wherein the multiple T cell epitopes are of the same self-antigen, such as comprised in the same self-antigen.

133. The tolerance-inducing construct of any one of embodiments 131-132, wherein the multiple T cell epitopes are of multiple different self-antigens, such as comprised in different self-antigens.

134. The tolerance-inducing construct of any of the preceding embodiments, wherein the antigenic unit comprises more than one T cell epitope, the antigenic unit comprises one or more linkers separating the T cell epitopes.

135. The tolerance-inducing construct of any of the preceding embodiments, wherein the antigenic unit comprises multiple antigens, such as multiple T cell epitopes of a self-antigen, an allergen, an alloantigen or a xenoantigen, wherein the T cell epitopes are preferably separated by linkers.

136. The tolerance-inducing construct of any of the preceding embodiments, wherein the antigenic unit comprises multiple T cell epitopes of a self-antigen, an allergen, an alloantigen or a xenoantigen, wherein each T cell epitope is separated from other T cell epitope by linkers.

137. The tolerance-inducing construct of any of the preceding embodiments, wherein an antigenic unit comprising n antigens comprises n-1 subunits, wherein each subunit comprises a T cell epitope of a self-antigen, an allergen, an alloantigen or a xenoantigen, and a linker, and further comprises a terminal T cell epitope.

138. The tolerance-inducing construct of embodiment 137, wherein n is an integer of from 1 to 50, e.g. 3 to 50 or 15 to 40 or 10 to 30 or 10 to 25 or 10 to 20 or 15 to 30 or 15 to 25 or 15 to 20.

139. The tolerance-inducing construct of any of the preceding embodiments, wherein the antigenic unit comprises a linker designed to be non-immunogenic.

140. The tolerance-inducing construct of any of embodiments 131-139, wherein the antigenic unit comprises one or more T cell epitopes of an allergen, such as one T cell epitope of an allergen or more than one T cell epitope of an allergen, such as multiple T cell epitopes of an allergen.

141. The tolerance-inducing construct of any of embodiments 131-140, wherein the multiple T cell epitopes are of the same allergen, such as comprised in the same allergen.

142. The tolerance-inducing construct of any of embodiments 131-141, wherein the multiple T cell epitopes are of multiple different allergens, i.e. comprised in different allergens.

143. The tolerance-inducing construct of any of the preceding embodiments, wherein the antigenic unit comprises one or more T cell epitopes of an alloantigen/xenoantigen, such as one T cell epitope of an alloantigen/xenoantigen or more than one T cell epitope of an alloantigen/xenoantigen, such as multiple T cell epitopes of an alloantigen/xenoantigen.

144. The tolerance-inducing construct of any of embodiments 131-143, wherein the multiple T cell epitopes are of the same alloantigen/xenoantigen, i.e. comprised in the same alloantigen/xenoantigen.

145. The tolerance-inducing construct of any of embodiments 131-144, wherein the multiple T cell epitopes are of multiple different alloantigen/xenoantigens, such as comprised in different alloantigens/xenoantigens.

146. The tolerance-inducing construct of any of the preceding embodiments, wherein the antigenic unit includes one T cell epitope.

147. The tolerance-inducing construct of any of the preceding embodiments, wherein the antigenic unit includes more than one T cell epitopes, such as multiple T cell epitopes.

148. The tolerance-inducing construct of any of the preceding embodiments, wherein the antigenic unit comprises T cell epitopes with a length of from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids, such as 25, 26, 27, 28, 29, 30, 31, 32, 33, 34, 35, 36, 37, 38, 39, 40, 41, 42, 43, 44, 45, 46, 47, 48, 49, or 50 amino acids.

149. The tolerance-inducing construct of any of embodiments 131-148, wherein the length of one T cell epitope is such that the protein does not fold correctly.

150. The tolerance-inducing construct of any of embodiments 131-149, wherein the T cell epitope has a length suitable for presentation by MHC (major histocompatibility complex).

151. The tolerance-inducing construct of any of the preceding embodiments, wherein the antigenic unit comprises T cell epitopes having a length suitable for specific presentation on MHC class I or MHC class II.

152. The tolerance-inducing construct of any of embodiments 131-151, wherein the T cell epitope has a length of from 7 to 11 amino acids for MHC class I presentation. In another embodiments, the T cell epitope sequence has a length of from 9- to 60 amino acids, such as from 9 to 30 amino acids, such as 15- to 60 amino acids, such as 15- to 30 amino acids for MHC class II presentation.

153. The tolerance-inducing construct of any of embodiments 131-152, wherein the T cell epitope has a length of 15 amino acids for MHC class II presentation.

154. The tolerance-inducing construct of any of the preceding embodiments, wherein the antigenic unit comprises up to 3500 amino acids, such as from 60 to 3500 amino acids, e.g. from about 80 or about 100 or about 150 amino acids to about a 3000 amino acids, such as from about 200 to about 2500 amino acids, such as from about 300 to about 2000 amino acids or from about 400 to about 1500 amino acids or from about 500 to about 1000 amino acids.

155. The tolerance-inducing construct of any of the preceding embodiments, wherein the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes.

156. The tolerance-inducing construct of any of embodiments 137-155, wherein the subunit antigenic unit comprises 1 to 3 T cell epitopes, such as 1, 2, 3, or 1 to 5 T cell epitopes, such as 1, 2, 3, 4, 5, or 3 to 6 T cell epitopes, such as 3, 4, 5, 6, or 5 to 15 T cell epitopes, such as 5, 6, 7, 8, 9, 10, 11, 12, 13, 14, or 15 T cell epitopes, or 7 to 17 T cell epitopes, such as 7, 8, 9, 10, 11, 12, 13, 14, 15, 16, or 17 T cell epitopes, or 9 to 19 T cell epitopes, such as 9, 10, 11, 12, 13, 14, 15, 16, 17, 18, or 19 T cell epitopes.

157. The tolerance-inducing construct of any of embodiments 131-156, wherein the T cell epitopes are randomly arranged in the antigenic unit.

158. The tolerance-inducing construct of any of embodiments 131-157, wherein the T cell epitopes are arranged in the order of more antigenic to less antigenic in the direction from multimerization/dimerization unit to the end of the antigenic unit.

159. The tolerance-inducing construct of any of embodiments 131-158, wherein the T cell epitopes are arranged in the order of more antigenic to less antigenic in the direction from the first joint region to the second joint region.

160. The tolerance-inducing construct of any of embodiments 131-158, wherein the most hydrophobic T cell epitopes are positioned substantially in the middle of the first antigenic unit and the most hydrophilic T cell epitopes are positioned towards the joint regions.

161. The tolerance-inducing construct of any of embodiments 131-158, wherein the T cell epitopes are arranged by alternating between a hydrophilic and a hydrophobic T cell epitope.

162. T The tolerance-inducing construct of any of embodiments 131-158, wherein GC rich sequences encoding T cell epitopes are arranged in such a way, that GC clusters are avoided.

163. The tolerance-inducing construct of embodiment 162, wherein GC rich T cell sequences are arranged such that there is at least one non-GC rich T cell sequence between them.

164. The tolerance-inducing construct of any of the preceding embodiments, wherein the construct is the polynucleotide, which further comprises a nucleotide sequence encoding a signal peptide.

165. The tolerance-inducing construct of any of the preceding embodiments, wherein the polynucleotide comprises a nucleotide sequence encoding a signal peptide that comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%, sequence identity to the amino acid sequence of SEQ ID NO: 6.

166. The tolerance-inducing construct of any of the preceding embodiments, wherein the polynucleotide comprises a nucleotide sequence encoding a signal peptide that comprises the amino acid sequence of SEQ ID NO: 6.

167. The tolerance-inducing construct of any of the preceding embodiments, wherein the polynucleotide comprises a nucleotide sequence encoding a signal peptide that consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% to the amino acid sequence of SEQ ID NO: 6.

168. The tolerance-inducing construct of any of the preceding embodiments, wherein the polynucleotide which comprises a nucleotide sequence encoding a signal peptide with the amino acid sequence of SEQ ID NO: 6.

169. The tolerance-inducing construct of any of the preceding embodiments, wherein the signal peptide comprises or consists of the amino acid sequence of SEQ ID NO: 6, wherein any one of the amino acids of the signal peptide has been substituted, deleted, or inserted for another amino acid, with the proviso that no more than 5 amino acids have been so substituted, deleted, or inserted, such as no more than 4 amino acids, such as no more than 3 amino acids, such as no more than 2 amino acids or no more than 1 amino acid.

170. A polynucleotide as defined in any of the preceding embodiments.

171. A vector comprising the polynucleotide according to embodiment 170.

172. A host cell comprising the polynucleotide according to embodiment and/or the vector according to any of embodiments 169-170.

173. A polypeptide encoded by the nucleotide sequence nucleic acid as defined in any of embodiments 1 to 169.

174. A multimeric protein, such as a dimeric protein, as defined in any of embodiments 1 to 169, wherein the multiple polypeptides, such as the two polypeptides, are linked to each other via their respective first joint regions and via their respective second joint regions.

175. A multimeric protein as defined in any of the embodiments 1 to 169, wherein the multiple polypeptides are linked to each other via their respective first joint regions and via their respective second joint regions.

176. A dimeric protein as defined in any of the preceding embodiments, wherein the two polypeptides are linked to each other via their respective first joint regions and via their respective second joint regions

177. A method of preparing a pharmaceutical composition, said method comprising:
a) providing the polynucleotide, the polypeptide or multimeric protein, such as the dimeric protein, according to any of embodiments 1 to 176; and
b) combining the polynucleotide, the polypeptide or the multimeric protein, such as the dimeric protein, with a pharmaceutically acceptable carrier.

178. A method of preparing a pharmaceutical composition, said method comprising:
a) providing the polynucleotide, the polypeptide or multimeric protein, such as dimeric protein, according to any of embodiments 1 to 176; and
b) combining the polynucleotide, the polypeptide or the multimeric protein, such as dimeric protein, with a pharmaceutically acceptable carrier.

179. A method of preparing a pharmaceutical composition, said method comprising:
a) providing the polynucleotide, the polypeptide or the dimeric protein according to any of embodiments 1 to 176; and
b) combining the polynucleotide, the polypeptide or the dimeric protein with a pharmaceutically acceptable carrier.

180. A pharmaceutical composition comprising the polynucleotide, the polypeptide or the multimeric protein, such as the dimeric protein, as defined in any of embodiments 1 to 176 and a pharmaceutically acceptable carrier.

181. A pharmaceutical composition comprising the polynucleotide, the polypeptide or the multimeric protein as defined in any of embodiments 1 to 175; and a pharmaceutically acceptable carrier.

182. A pharmaceutical composition comprising the polynucleotide, the polypeptide or the dimeric protein as defined in any of embodiments 1 to 176; and a pharmaceutically acceptable carrier.

183. A pharmaceutical composition comprising the polynucleotide of any one of embodiment 1 to 176, further comprising one or more pharmaceutically acceptable excipients and/or diluents.

184. A pharmaceutical composition comprising the polynucleotide according to any one of embodiments 1 to 176, wherein the pharmaceutically acceptable carrier is selected from the group consisting of saline, buffered saline, PBS, dextrose, water, glycerol, ethanol, sterile isotonic aqueous buffers, and combin157ations thereof.

185. The pharmaceutical composition of embodiments 180-185 for use as a medicament.

186. The pharmaceutical composition of embodiments 180-185 for use in in the treatment of conditions involving undesired immune reactions, such as in the prophylactic or therapeutic treatment of autoimmune diseases, allergic disease and graft rejection.

187. The pharmaceutical composition of embodiments 180-186 for use in the treatment of an autoimmune disease.

188. The pharmaceutical composition of embodiments 180-187 for use in the treatment of an allergy.

189. The pharmaceutical composition of embodiments 180-188 for use in the treatment of graft rejection.

190. A method for treating a subject having or suspected of having an immune disease selected from the group consisting of autoimmune disease, allergic disease and graft rejection, or being in need of prevention thereof, the method comprising administering to the subject a pharmaceutical composition of any one of embodiments 180-189 comprising a pharmaceutically acceptable carrier.

191. A pharmaceutical composition for use in the prophylactic or therapeutic treatment of an immune disease selected from the group consisting of autoimmune disease, allergic disease and graft rejection, wherein the pharmaceutical composition is the pharmaceutical composition of any one embodiments 180-189.

192. A pharmaceutical composition for use in the prophylactic or therapeutic treatment of a subject suffering or suspected of suffering from an immune disease selected from the group consisting of autoimmune disease, allergic disease and graft rejection, wherein the pharmaceutical composition is the pharmaceutical composition of any one embodiments 180-189.

193. Use of a pharmaceutical composition for the prophylactic or therapeutic treatment of an immune disease selected from the group consisting of autoimmune disease, allergic disease and graft rejection, wherein the pharmaceutical composition is the pharmaceutical composition of any one embodiments 180-189.

194. Use of a pharmaceutical composition for the manufacture of a medicament for the prophylactic or therapeutic treatment of a subject suffering or suspected of suffering from an immune disease selected from the group consisting of autoimmune disease, allergic disease and graft rejection, wherein the pharmaceutical composition is the pharmaceutical composition of any one embodiments 180-189.

195. Use of a pharmaceutical composition for prophylactically or therapeutically treating a subject having an immune disease selected from the group consisting of autoimmune disease, allergic disease and graft rejection, wherein the pharmaceutical composition is the pharmaceutical composition of any one embodiments 180-189.

196. Use of a pharmaceutical composition of any one embodiments 180-189 for the manufacture of a medicament for the prophylactic or therapeutic treatment of a subject having or suspected of having an immune disease selected from the group consisting of autoimmune disease, allergic disease and graft rejection, wherein the medicament is administered to said subject.

197. Use of a pharmaceutical composition of any one embodiments 180-189 for the prophylactic or therapeutic treatment of subject having or suspected of having an immune disease selected from the group consisting of autoimmune disease, allergic disease and graft rejection, wherein the medicament is administered to said subject.

198. A pharmaceutical composition of any one embodiments 180-189 when used in the prophylactic or therapeutic treatment of an immune disease selected from the group consisting of autoimmune disease, allergic disease and graft rejection.

199. A medicament for the prophylactic or therapeutic treatment of a subject having or suspected of having an immune disease selected from the group consisting of autoimmune disease, allergic disease and graft rejection by administering to the subject a pharmaceutical composition of any one embodiments 180-189.

200. A method of preparing a polypeptide or a multimeric protein, such as a dimeric protein, said method comprising:
a) transfecting a cell with the vector as defined in embodiment 171 or the polynucleotide according to any of embodiments 1 to 170;
b) culturing the cell, whereby the cell expresses a polypeptide encoded by said polynucleotide; and
c) obtaining and purifying the multimeric protein, such as the dimeric protein, and/or the polypeptide expressed by the cell.

201. A method of preparing a polypeptide or a multimeric protein, said method comprising:
a) transfecting a cell with the vector as defined in embodiments 171 or the polynucleotide according to any of embodiments 1 to 170 and 173;
b) culturing the cell, whereby the cell expresses a polypeptide encoded by said polynucleotide; and
c) obtaining and purifying the multimeric protein and/or the polypeptide expressed by the cell.

202. A method of preparing a polypeptide or a dimeric protein, said method comprising:
a) transfecting a cell with the vector as defined in embodiments 171 or the polynucleotide according to any of embodiments 1 to 170 and 173;
b) culturing the cell, whereby the cell expresses a polypeptide encoded by said polynucleotide; and
c) obtaining and purifying the dimeric protein, and/or the polypeptide expressed by the cell.

203. The method according to any of embodiments 200-202, wherein step c) comprises the step of purifying the fraction containing the multiple protein, such as the dimeric protein, wherein multiple polypeptides, such as two polypeptides, are linked to each other via their respective first joint regions and via their respective second joint regions.

204. A method for treating conditions involving undesired immune reactions, such as in the prophylactic or therapeutic treatment of autoimmune diseases, allergic disease and graft rejection, said method comprising administering the polynucleotide, the polypeptide, the multimeric protein or the dimeric protein according to any of embodiments 1 to 170 and 173, the vector according to embodiments 171 or the pharmaceutical composition according to any one of embodiments 180 to 189, to a subject in need thereof.

205. The method according to embodiments 204, wherein the subject is a mammal.

206. The method according to embodiments 205, wherein the mammal is a human.

1.-50. (canceled)

51. A tolerance-inducing construct comprising:
i) a polynucleotide comprising a nucleotide sequence encoding a polypeptide, the polypeptide comprising, in the specified order:
a. a first targeting unit, a first joint region;
b. an antigenic unit;
c. a second joint region; and
d. a second targeting unit;
wherein the antigenic unit comprises one or more T cell epitopes of a self-antigen, an allergen, an alloantigen or a xenoantigen; or
ii) a polypeptide encoded by the nucleotide sequence as defined in i); or
iii) a multimeric protein, such as a dimeric protein consisting of multiple polypeptides as defined in ii), such as two polypeptides.

52. The tolerance-inducing construct of claim 51, wherein the multimeric protein, such as the dimeric protein, consists of multiple polypeptides, such as two polypeptides, that are linked to each other via their joint regions, for example via their respective first joint regions and via their respective second joint regions.

53. The tolerance-inducing construct of claim 51, wherein the first- and second joint regions comprise a flexible unit and a binding unit, wherein the binding unit is either a non-covalent binding unit, for example wherein the non-covalent binding unit comprises or consists of a trimerization unit, such as the C-terminal domain of T4 fibritin or such as a collagen-derived trimerization unit, such as human collagen derived XVIII trimerization domain or human collagen XV trimerization domain, or a tetramerization unit, such as a domain derived from p53, or comprises or consists of a dimerization unit, such as a dimerization unit comprising a hinge region and an immunoglobulin domain, e.g. an immunoglobulin constant domain or a dimerization unit comprising the dHLX protein, or a covalent binding unit.

54. The tolerance-inducing construct of claim 51, wherein the first and second joint regions comprise a covalent binding unit which comprises cysteine residues, such as at least 2 cysteine residues or a cysteine rich sequence,
for example wherein the covalent binding unit of the first joint region comprises a different number of cysteine residues than the covalent binding unit of the second joint region,
and/or wherein the cysteine residues comprised in the covalent binding unit of the first joint region are positioned differently than the cysteine residues comprised in the covalent binding unit of the second joint region, such as wherein the number of amino acid residues between the cysteine residues of the covalent binding unit of the first joint region is different than that of the second joint region.

55. The tolerance-inducing construct of claim 53, wherein at least one of the covalent binding units is derived from an immunoglobulin, such as a hinge region derived from an immunoglobulin, such as exon h4 of IgG3 or the middle hinge of IgG1,
and/or wherein at least one of the covalent binding units is an artificial sequence.

56. The tolerance-inducing construct of claim 51, wherein the first and second joint regions comprise a non-covalent binding unit,
for example wherein the non-covalent binding unit is or comprises (a) an immunoglobulin, or is derived from an immunoglobulin such as a CH3 domain derived from IgG3 or from IgG1,
or (b) comprises a leucine zipper motif, a Jun/Fos-based leucine zipper, or is derived from the bZIP class of eukaryotic transcription factors, such as the amino acid sequence of SEQ ID NO: 5.

57. The tolerance-inducing construct of claim 51, wherein the joint regions comprise a binding unit which comprises a covalent binding unit and a non-covalent binding unit.

58. The tolerance-inducing construct of claim 53, wherein at least one of the flexible units is a naturally occurring peptide sequence,
such as derived from an immunoglobulin, such as a hinge region derived from an immunoglobulin, such as exon h1 of IgG3 or the lower hinge of IgG1,
and/or wherein at least one of the flexible units is an artificial sequence,
such as a glycine-serine linker, such as GGGGSGGGGS (SEQ ID NO: 80).

59. The tolerance-inducing construct of claim 51, wherein at least one of the first- or the second targeting units comprises a moiety that interacts with surface molecules on antigen-presenting cells, for example wherein both the first and the second targeting unit comprises a moiety that interacts with surface molecules on antigen-presenting cells.

60. The tolerance-inducing construct of claim 59, wherein the surface molecules are present on the same cell and/or wherein said interaction is binding and wherein binding of the first- or the second targeting unit causes internalization of the construct.

61. The tolerance-inducing construct of claim 59, wherein the surface molecule is selected from the group consisting of TGFβ receptor (TGFβR1, TGFβR2, or TGFβR3), IL10R, such as IL-10RA and IL10-RB, IL2R, IL4R, IL6R, IL11R and IL13R, IL27R, IL35R, IL37R, CCR7, CD11b, CD11c, CD103, CD14, CD36, CD205, CD109, VISTA, MARCO, MHCII, MHCII, CD83, SIGLEC, MGL, CD80, CD86, Clec9A, Clec12A, Clec12B, DCIR2, Langerin, MR, DC-Sign, Treml4, Dectin-1, PDL1, PDL2, HVEM, arylhydrocarbon receptor and vitamin D receptor.

62. The tolerance-inducing construct of claim 61, wherein the targeting unit comprises a moiety which is a natural ligand, an antibody or part thereof, e.g. a scFv, or a synthetic ligand,
for example wherein the targeting unit comprises a natural ligand which is selected from the group consisting of TGFβ, IL-10, ILIRA, IL2, IL4, IL6, IL11, IL13, IL27, IL35, IL37, CCL19, CCL21, ICAM-1 (Intercellular Adhesion Molecule 1 also known as CD54), keratin, VSIG-3, SCGB3A2, CTLA-4, for example the extracellular domain of CTLA-4, PD-1, for example the extracellular domain of PD-1 and BTLA, for example the extracellular domain of BTLA.

63. The tolerance-inducing construct of claim 51, wherein the first- and the second targeting units are identical, or wherein the first- and the second targeting units are different.

64. The tolerance-inducing construct of claim 51, wherein the construct is the polynucleotide, which further comprises a nucleotide sequence encoding a signal peptide.

65. A multimeric protein, such as a dimeric protein, as defined in claim 51, wherein the multiple polypeptides, such as the two polypeptides, are linked to each other via their respective first joint regions and via their respective second joint regions.

66. A method of preparing a pharmaceutical composition, said method comprising:
a) providing the polynucleotide, the polypeptide or the multimeric protein,
such as the dimeric protein, as defined in claim 51; and
b) combining the polynucleotide, the polypeptide or the multimeric protein,
such as the dimeric protein, with a pharmaceutically acceptable carrier.

67. A pharmaceutical composition comprising the polynucleotide, the polypeptide or the multimeric protein, such as the dimeric protein, as defined in claim 51 and a pharmaceutically acceptable carrier.

68. A vector comprising the polynucleotide as defined in claim 51.

69. A host cell comprising the vector of claim 68.

70. A method for treating conditions involving undesired immune reactions, such as in the prophylactic or therapeutic treatment of autoimmune diseases, allergic disease and graft rejection, said method comprising administering the pharmaceutical composition as defined in claim 67 to a subject in need thereof.


SEQUENCE LISTING (171 sequences)

SEQ ID NO: 1 - protein, 27 aa, Artificial Sequence
  Synthetic sequence
  MISC_FEATURE (1)..(12): Amino acid sequence of hinge exon h1 from IgG3
  MISC_FEATURE (13)..(27): Amino acid sequence of and hinge exon h4 from IgG3
ELKTPLGDTTHTEPKSCDTPPPCPRCP

SEQ ID NO: 2 - protein, 23 aa, Artificial Sequence
  Synthetic sequence
  MISC_FEATURE (1)..(23): Amino acid sequence of hinge regions of IgG1
EPKSCDKTHTCPPCPAPELLGGP

SEQ ID NO: 3 - protein, 107 aa, Artificial Sequence
  Synthetic sequence
  MISC_FEATURE (1)..(107): Amino acid sequence of the CH3 domain of human IgG3
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 107 aa, Artificial Sequence
  Synthetic sequence
  MISC_FEATURE (1)..(107): Amino acid sequence of the CH3 domain of human IgG1
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 29 aa, Artificial Sequence
  Synthetic sequence
  MISC_FEATURE (1)..(29): Amino acid sequence of the CREB bZIP motif
VKCLENRVAVLENQNKTLIEELKALKDLY

SEQ ID NO: 6 - protein, 19 aa, Artificial Sequence
  Synthetic sequence
  MISC_FEATURE (1)..(19): Mouse immunoglobulin heavy chain signal sequence (Ig VH signal seq)
MNFGLRLIFLVLTLKGVQC

SEQ ID NO: 7 - protein, 246 aa, Artificial Sequence
  Synthetic sequence
  MISC_FEATURE (1)..(246): Mouse single chain variable fragment (scFv) anti-DEC205
DIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSS

SEQ ID NO: 8 - protein, 20 aa, Artificial Sequence
  Synthetic sequence
  MISC_FEATURE (1)..(20): Hinge region form human IgG1
GLQGLEPKSCDKTHTCPPCP

SEQ ID NO: 9 - protein, 160 aa, Artificial Sequence
  Synthetic sequence
  MISC_FEATURE (1)..(160): Murine IL-10
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 10 - protein, 112 aa, Artificial Sequence
  Synthetic sequence
  MISC_FEATURE (1)..(112): Mature murine TGFbeta1
ALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 11 - protein, 126 aa, Artificial Sequence
  Synthetic sequence
  MISC_FEATURE (1)..(126): Mature murine CTLA-4
EAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSD

SEQ ID NO: 12 - protein, 37 aa, Artificial Sequence
  Synthetic sequence
  MISC_FEATURE (1)..(37): MOG(27-63)
SPGKNATGMEVGWYRSPFSRVVHLYRNGKDQDAEQAP

SEQ ID NO: 13 - protein, 37 aa, Artificial Sequence
  Synthetic sequence
  MISC_FEATURE (1)..(37): MOG(27-63) sequence obtained from Krienke et al. 2021. Patent US2020061166A1
SPGKNATGMEVGWYRSPFSRVVHLYRNGKDQDAEAQP

SEQ ID NO: 14 - protein, 21 aa, Artificial Sequence
  Synthetic sequence
  MISC_FEATURE (1)..(21): MOG(35-55)
MEVGWYRSPFSRVVHLYRNGK

SEQ ID NO: 15 - protein, 21 aa, Artificial Sequence
  Synthetic sequence
  MISC_FEATURE (1)..(21): Murine MARCO ligand SCGB3A2 signal sequence
MKLVSIFLLVTIGICGYSATA

SEQ ID NO: 16 - protein, 70 aa, Artificial Sequence
  Synthetic sequence
  MISC_FEATURE (1)..(70): Murine MARCO ligand SCGB3A2
LLINRLPVVDKLPVPLDDIIPSFDPLKMLLKTLGISVEHLVTGLKKCVDELGPEASEAVKKLLEALSHLV

SEQ ID NO: 17 - protein, 22 aa, Artificial Sequence
  Synthetic sequence
  MISC_FEATURE (1)..(22): Murine VISTA ligand VSIG-3 signal sequence
MTRRRSAPASWLLVSLLGVATS

SEQ ID NO: 18 - protein, 218 aa, Artificial Sequence
  Synthetic sequence
  MISC_FEATURE (1)..(218): Murine VISTA ligand VSIG-3 extracellular domain
LEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSV

SEQ ID NO: 19 - protein, 12 aa, Artificial Sequence
  Synthetic sequence
  MISC_FEATURE (1)..(12): Hinge h1 hIgG3
ELKTPLGDTTHT

SEQ ID NO: 20 - protein, 23 aa, Artificial Sequence
  Synthetic sequence
  MISC_FEATURE (1)..(23): Human CCL3L1 signal sequence
MQVSTAALAVLLCTMALCNQVLS

SEQ ID NO: 21 - protein, 70 aa, Artificial Sequence
  Synthetic sequence
  MISC_FEATURE (1)..(70): Human CCL3L1
APLAADTPTACCFSYTSRQIPQNFIADYFETSSQCSKPSVIFLTKRGRQVCADPSEEWVQKYVSDLELSA

SEQ ID NO: 22 - protein, 171 aa, Artificial Sequence
  Synthetic sequence
  MISC_FEATURE (1)..(171): Met e 1 (241-260), (210-230), (136-155), (76-95), (46-65), (16-35)
KEVDRLEDELVNEKEKYKSIGGGGSGGGGSAYKEQIKTLTNKLKAAEARAEGGGGSGGGGSNQLKEARFLAEEADRKYDEVGGGGSGGGGSAALNRRIQLLEEDLERSEERGGGGSGGGGSDLDQVQESLLKANNQLVEKDGGGGSGGGGSEQQNKEANNRAEKSEEEVHN

SEQ ID NO: 23 - protein, 514 aa, Artificial Sequence
  Synthetic sequence
  MISC_FEATURE (1)..(514): Amino acid sequence of VB5050.
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLSPGKNATGMEVGWYRSPFSRVVHLYRNGKDQDAEQAPGLQGLEPKSCDKTHTCPPCPSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 24 - protein, 514 aa, Artificial Sequence
  Synthetic sequence
  MISC_FEATURE (1)..(514): Amino acid sequence of VB5038
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLSPGKNATGMEVGWYRSPFSRVVHLYRNGKDQDAEAQPGLQGLEPKSCDKTHTCPPCPSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 25 - protein, 526 aa, Artificial Sequence
  Synthetic sequence
  MISC_FEATURE (1)..(526): Amino acid sequence of VB5042
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLSPGKNATGMEVGWYRSPFSRVVHLYRNGKDQDAEQAPGLQGLEPKSCDKTHTCPPCPELKTPLGDTTHTSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 26 - protein, 466 aa, Artificial Sequence
  Synthetic sequence
  MISC_FEATURE (1)..(466): Amino acid sequence of VB5066
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLSPGKNATGMEVGWYRSPFSRVVHLYRNGKDQDAEQAPGLQGLEPKSCDKTHTCPPCPALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 27 - protein, 478 aa, Artificial Sequence
  Synthetic sequence
  MISC_FEATURE (1)..(478): Amino acid sequence of VB5043
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLSPGKNATGMEVGWYRSPFSRVVHLYRNGKDQDAEQAPGLQGLEPKSCDKTHTCPPCPELKTPLGDTTHTALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 28 - protein, 480 aa, Artificial Sequence
  Synthetic sequence
  MISC_FEATURE (1)..(480): Amino acid sequence of VB5067
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLSPGKNATGMEVGWYRSPFSRVVHLYRNGKDQDAEQAPGLQGLEPKSCDKTHTCPPCPEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSD

SEQ ID NO: 29 - protein, 340 aa, Artificial Sequence
  Synthetic sequence
  MISC_FEATURE (1)..(340): Amino acid sequence of VB5072
MKLVSIFLLVTIGICGYSATALLINRLPVVDKLPVPLDDIIPSFDPLKMLLKTLGISVEHLVTGLKKCVDELGPEASEAVKKLLEALSHLVELKTPLGDTTHTEPKSCDTPPPCPRCPGLGGLSPGKNATGMEVGWYRSPFSRVVHLYRNGKDQDAEQAPGLQGLEPKSCDKTHTCPPCPSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 30 - protein, 352 aa, Artificial Sequence
  Synthetic sequence
  MISC_FEATURE (1)..(352): Amino acid sequence of VB5073
MKLVSIFLLVTIGICGYSATALLINRLPVVDKLPVPLDDIIPSFDPLKMLLKTLGISVEHLVTGLKKCVDELGPEASEAVKKLLEALSHLVELKTPLGDTTHTEPKSCDTPPPCPRCPGLGGLSPGKNATGMEVGWYRSPFSRVVHLYRNGKDQDAEQAPGLQGLEPKSCDKTHTCPPCPELKTPLGDTTHTSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 31 - protein, 489 aa, Artificial Sequence
  Synthetic sequence
  MISC_FEATURE (1)..(489): Amino acid sequence of VB5074
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSVELKTPLGDTTHTEPKSCDTPPPCPRCPGLGGLSPGKNATGMEVGWYRSPFSRVVHLYRNGKDQDAEQAPGLQGLEPKSCDKTHTCPPCPSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 32 - protein, 501 aa, Artificial Sequence
  Synthetic sequence
  MISC_FEATURE (1)..(501): Amino acid sequence of VB5075
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSVELKTPLGDTTHTEPKSCDTPPPCPRCPGLGGLSPGKNATGMEVGWYRSPFSRVVHLYRNGKDQDAEQAPGLQGLEPKSCDKTHTCPPCPELKTPLGDTTHTSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 33 - protein, 279 aa, Artificial Sequence
  Synthetic sequence
  MISC_FEATURE (1)..(279): Amino acid sequence of VB5052
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 34 - protein, 279 aa, Artificial Sequence; identical to SEQ ID NO: 33
  Synthetic sequence
  MISC_FEATURE (1)..(279): Amino acid sequence of VB5002b

SEQ ID NO: 35 - protein, 56 aa, Artificial Sequence
  Synthetic sequence
  MISC_FEATURE (1)..(56): Amino acid sequence of VB5051
MNFGLRLIFLVLTLKGVQCSPGKNATGMEVGWYRSPFSRVVHLYRNGKDQDAEQAP

SEQ ID NO: 36 - protein, 56 aa, Artificial Sequence
  Synthetic sequence
  MISC_FEATURE (1)..(56): Amino acid sequence of VB5001b
MNFGLRLIFLVLTLKGVQCSPGKNATGMEVGWYRSPFSRVVHLYRNGKDQDAEAQP

SEQ ID NO: 37 - protein, 648 aa, Artificial Sequence
  Synthetic sequence
  MISC_FEATURE (1)..(648): Amino acid sequence of VB5077
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLKEVDRLEDELVNEKEKYKSIGGGGSGGGGSAYKEQIKTLTNKLKAAEARAEGGGGSGGGGSNQLKEARFLAEEADRKYDEVGGGGSGGGGSAALNRRIQLLEEDLERSEERGGGGSGGGGSDLDQVQESLLKANNQLVEKDGGGGSGGGGSEQQNKEANNRAEKSEEEVHNGLQGLEPKSCDKTHTCPPCPSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 38 - protein, 660 aa, Artificial Sequence
  Synthetic sequence
  MISC_FEATURE (1)..(660): Amino acid sequence of VB5078
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLKEVDRLEDELVNEKEKYKSIGGGGSGGGGSAYKEQIKTLTNKLKAAEARAEGGGGSGGGGSNQLKEARFLAEEADRKYDEVGGGGSGGGGSAALNRRIQLLEEDLERSEERGGGGSGGGGSDLDQVQESLLKANNQLVEKDGGGGSGGGGSEQQNKEANNRAEKSEEEVHNGLQGLEPKSCDKTHTCPPCPELKTPLGDTTHTSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 39 - protein, 6 aa, Artificial Sequence
  Synthetic sequence
  MISC_FEATURE (1)..(6): linker
  MISC_FEATURE (1)..(4): The sequence can be repeated m times, wherein m is an integer from 1 to 5, e.g., 1, 2, 3, 4, or 5.
EAAKGS

SEQ ID NO: 40 - protein, 5 aa, Artificial Sequence
  Synthetic sequence
  MISC_FEATURE (1)..(5): linker
GLSGL

SEQ ID NO: 41 - protein, 510 aa, Artificial Sequence
  Synthetic sequence
  MISC_FEATURE (1)..(510): Amino acid sequence of VB5041
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGLGGLMEVGWYRSPFSRVVHLYRNGKGLQGLEPKSCDKTHTCPPCPELKTPLGDTTHTSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 42 - protein, 36 aa, Artificial Sequence
  Synthetic sequence
  MISC_FEATURE (1)..(36): linker
RTQDENPVVHFFKNIVTPRTPPPSQGKGRGLSLSRF

SEQ ID NO: 43 - protein, 18 aa, Artificial Sequence
  Synthetic sequence
  MISC_FEATURE (1)..(18): Natural leader sequence murine IL-10
MPGSALLCCLLLLTGMRI

SEQ ID NO: 44 - protein, 35 aa, Artificial Sequence
  Synthetic sequence
  MISC_FEATURE (1)..(35): Natural leader sequence murine CTLA4
MACLGLRRYKAQLQLPSRTWPFVALLTLLFIPVFS

SEQ ID NO: 45 - protein, 29 aa, Artificial Sequence
  Synthetic sequence
  MISC_FEATURE (1)..(29): Natural leader sequence murine transforming growth factor beta-1
MPPSGLRLLPLLLPLPWLLVLTPGRPAAG

SEQ ID NO: 46 - protein, 361 aa, Artificial Sequence
  Synthetic sequence
  MISC_FEATURE (1)..(361): Murine transforming growth factor beta-1 proprotein
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 47 - protein, 24 aa, Artificial Sequence
  Synthetic sequence
  MISC_FEATURE (1)..(24): Natural leader sequence murine PD-1
MWVRQVPWSFTWAVLQLSWQSGWL

SEQ ID NO: 48 - protein, 145 aa, Artificial Sequence
  Synthetic sequence
  MISC_FEATURE (1)..(145): Murine PD-1 Extracellular domain
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 49 - protein, 20 aa, Artificial Sequence
  Synthetic sequence
  MISC_FEATURE (1)..(20): GAD65 (202-221)
TNMFTYEIAPVFVLLEYVTL

SEQ ID NO: 50 - protein, 20 aa, Artificial Sequence
  Synthetic sequence
  MISC_FEATURE (1)..(20): Mete1 (16-35)
EQQNKEANNRAEKSEEEVHN

SEQ ID NO: 51 - protein, 20 aa, Artificial Sequence
  Synthetic sequence
  MISC_FEATURE (1)..(20): Mete1(46-65)
DLDQVQESLLKANNQLVEKD

SEQ ID NO: 52 - protein, 20 aa, Artificial Sequence
  Synthetic sequence
  MISC_FEATURE (1)..(20): Met e 1(76-95)
AALNRRIQLLEEDLERSEER

SEQ ID NO: 53 - protein, 20 aa, Artificial Sequence
  Synthetic sequence
  MISC_FEATURE (1)..(20): Met e 1(136-155)
NQLKEARFLAEEADRKYDEV

SEQ ID NO: 54 - protein, 21 aa, Artificial Sequence
  Synthetic sequence
  MISC_FEATURE (1)..(21): Met e 1(210-230)
AYKEQIKTLTNKLKAAEARAE

SEQ ID NO: 55 - protein, 20 aa, Artificial Sequence
  Synthetic sequence
  MISC_FEATURE (1)..(20): Met e 1(241-260)
KEVDRLEDELVNEKEKYKSI

SEQ ID NO: 56 - protein, 21 aa, Artificial Sequence
  Synthetic sequence
  MISC_FEATURE (1)..(21): linker
VHFFKNIVTPRTPPPSQGKGR

SEQ ID NO: 57 - protein, 30 aa, Artificial Sequence
  Synthetic sequence
  MISC_FEATURE (1)..(30): linker
AVPVYIYFNTWTTCQSIAFPSKTSASIGSL

SEQ ID NO: 58 - protein, 14 aa, Artificial Sequence
  Synthetic sequence
  MISC_FEATURE (1)..(14): linker
NTWTTCQSIAFPSK

SEQ ID NO: 59 - DNA, 1173 bp, Artificial Sequence
  Synthetic sequence
  misc_feature (1)..(1173): Human transforming growth factor beta-1
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 60 - protein, 390 aa, Artificial Sequence
  Synthetic sequence
  MISC_FEATURE (1)..(390): Human transforming growth factor beta-1
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 61 - DNA, 1329 bp, Artificial Sequence
  Synthetic sequence
  misc_feature (1)..(1329): Human transforming growth factor beta 2 (TGFB2)
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 62 - protein, 442 aa, Artificial Sequence
  Synthetic sequence
  MISC_FEATURE (1)..(442): Human Transforming growth factor beta-2
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 63 - DNA, 930 bp, Artificial Sequence
  Synthetic sequence
  misc_feature (1)..(930): Human transforming growth factor beta 3 (TGFB3)
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtga

SEQ ID NO: 64 - protein, 309 aa, Artificial Sequence
  Synthetic sequence
  MISC_FEATURE (1)..(309): Human transforming growth factor beta-3
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFR

SEQ ID NO: 65 - DNA, 537 bp, Artificial Sequence
  Synthetic sequence
  misc_feature (1)..(537): Human interleukin 10 (IL10)
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 66 - protein, 178 aa, Artificial Sequence
  Synthetic sequence
  MISC_FEATURE (1)..(178): Human interleukin-10 isoform 1 precursor
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 67 - DNA, 282 bp, Artificial Sequence
  Synthetic sequence
  misc_feature (1)..(282): Human secretoglobin family 3A member 2 (SCGB3A2)
atgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggaggcgctatcacacttggtgtga

SEQ ID NO: 68 - protein, 93 aa, Artificial Sequence
  Synthetic sequence
  MISC_FEATURE (1)..(93): Human secretoglobin family 3A member 2 precursor
MKLVTIFLLVTISLCSYSATAFLINKVPLPVDKLAPLPLDNILPFMDPLKLLLKTLGISVEHLVEGLRKCVNELGPEASEAVKKLLEALSHLV

SEQ ID NO: 69 - DNA, 723 bp, Artificial Sequence
  Synthetic sequence
  misc_feature (1)..(723): Human immunoglobulin superfamily member 11 (IGSF11) Extracellular domain
atgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattgga

SEQ ID NO: 70 - protein, 241 aa, Artificial Sequence
  Synthetic sequence
  MISC_FEATURE (1)..(431): Human immunoglobulin superfamily member 11 i (IGSF11) Extracellular domain
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 71 - DNA, 483 bp, Artificial Sequence
  Synthetic sequence
  misc_feature (1)..(483): Human cytotoxic T-lymphocyte associated protein 4 (CTLA4) Extracellular Domain
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgac

SEQ ID NO: 72 - protein, 161 aa, Artificial Sequence
  Synthetic sequence
  MISC_FEATURE (1)..(161): CTLA4 HUMAN Cytotoxic T-lymphocyte protein 4 Extracellular Domain
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 73 - DNA, 510 bp, Artificial Sequence
  Synthetic sequence
  misc_feature (1)..(510): Human programmed cell death 1 (PDCD1) extracellular domain
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg

SEQ ID NO: 74 - protein, 170 aa, Artificial Sequence
  Synthetic sequence
  MISC_FEATURE (1)..(170): Human programmed cell death protein 1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 75 - protein, 10 aa, Artificial Sequence
  Synthetic sequence
  MISC_FEATURE (1)..(10): linker
GGGGSGGGSS

SEQ ID NO: 76 - protein, 5 aa, Artificial Sequence
  Synthetic sequence
  MISC_FEATURE (1)..(5): linker
GGGSG

SEQ ID NO: 77 - protein, 5 aa, Artificial Sequence
  Synthetic sequence
  MISC_FEATURE (1)..(5): linker
GGSGG

SEQ ID NO: 78 - protein, 6 aa, Artificial Sequence
  Synthetic sequence
  MISC_FEATURE (1)..(6): linker
SGSSGS

SEQ ID NO: 79 - protein, 5 aa, Artificial Sequence
  Synthetic sequence
  MISC_FEATURE (1)..(5): linker
GGGGS

SEQ ID NO: 80 - protein, 10 aa, Artificial Sequence
  Synthetic sequence
  MISC_FEATURE (1)..(10): linker
GGGGSGGGGS

SEQ ID NO: 81 - protein, 5 aa, Artificial Sequence
  Synthetic sequence
  MISC_FEATURE (1)..(5): linker
GGGGS

SEQ ID NO: 82 - protein, 5 aa, Artificial Sequence
  Synthetic sequence
  MISC_FEATURE (1)..(5): linker
  MISC_FEATURE (1)..(5): The sequence can be repeated m times, wherein m is an integer from 1 to 5, e.g., 1, 2, 3, 4, or 5.
GGGSS

SEQ ID NO: 83 - protein, 5 aa, Artificial Sequence
  Synthetic sequence
  MISC_FEATURE (1)..(5): linker
  MISC_FEATURE (1)..(5): The sequence can be repeated m times, wherein m is an integer from 1 to 5, e.g., 1, 2, 3, 4, or 5.
GGGSG

SEQ ID NO: 84 - protein, 6 aa, Artificial Sequence
  Synthetic sequence
  MISC_FEATURE (1)..(6): linker
  MISC_FEATURE (1)..(6): The sequence can be repeated m times, wherein m is an integer from 1 to 5, e.g., 1, 2, 3, 4, or 5.
SGSSGS

SEQ ID NO: 85 - protein, 5 aa, Artificial Sequence
  Synthetic sequence
  MISC_FEATURE (1)..(5): linker
LGGGS

SEQ ID NO: 86 - protein, 5 aa, Artificial Sequence
  Synthetic sequence
  MISC_FEATURE (1)..(5): linker
GLGGS

SEQ ID NO: 87 - protein, 5 aa, Artificial Sequence
  Synthetic sequence
  MISC_FEATURE (1)..(5): linker
GGLGS

SEQ ID NO: 88 - protein, 5 aa, Artificial Sequence
  Synthetic sequence
  MISC_FEATURE (1)..(5): linker
GGGLS

SEQ ID NO: 89 - protein, 5 aa, Artificial Sequence
  Synthetic sequence
  MISC_FEATURE (1)..(5): linker
GGGGL

SEQ ID NO: 90 - protein, 5 aa, Artificial Sequence
  Synthetic sequence
  MISC_FEATURE (1)..(5): linker
LGGSG

SEQ ID NO: 91 - protein, 5 aa, Artificial Sequence
  Synthetic sequence
  MISC_FEATURE (1)..(5): linker
GLGSG

SEQ ID NO: 92 - protein, 5 aa, Artificial Sequence
  Synthetic sequence
  MISC_FEATURE (1)..(5): linker
GGLSG

SEQ ID NO: 93 - protein, 5 aa, Artificial Sequence
  Synthetic sequence
  MISC_FEATURE (1)..(5): linker
GGGLG

SEQ ID NO: 94 - protein, 5 aa, Artificial Sequence
  Synthetic sequence
  MISC_FEATURE (1)..(5): linker
GGGSL

SEQ ID NO: 95 - protein, 5 aa, Artificial Sequence
  Synthetic sequence
  MISC_FEATURE (1)..(5): linker
LGGSS

SEQ ID NO: 96 - protein, 5 aa, Artificial Sequence
  Synthetic sequence
  MISC_FEATURE (1)..(5): linker
GLGSS

SEQ ID NO: 97 - protein, 5 aa, Artificial Sequence
  Synthetic sequence
  MISC_FEATURE (1)..(5): linker
GGLSS

SEQ ID NO: 98 - protein, 5 aa, Artificial Sequence
  Synthetic sequence
  MISC_FEATURE (1)..(5): linker
LGLGS

SEQ ID NO: 99 - protein, 5 aa, Artificial Sequence
  Synthetic sequence
  MISC_FEATURE (1)..(5): linker
GLGLS

SEQ ID NO: 100 - protein, 5 aa, Artificial Sequence
  Synthetic sequence
  MISC_FEATURE (1)..(5): linker
GLLGS

SEQ ID NO: 101 - protein, 5 aa, Artificial Sequence
  Synthetic sequence
  MISC_FEATURE (1)..(5): linker
LGGLS

SEQ ID NO: 102 - protein, 5 aa, Artificial Sequence
  Synthetic sequence
  MISC_FEATURE (1)..(5): linker
GLGGL

SEQ ID NO: 103 - protein, 5 aa, Artificial Sequence
  Synthetic sequence
  MISC_FEATURE (1)..(5): linker
LGLSG

SEQ ID NO: 104 - protein, 5 aa, Artificial Sequence
  Synthetic sequence
  MISC_FEATURE (1)..(5): linker
GLLSG

SEQ ID NO: 105 - protein, 5 aa, Artificial Sequence
  Synthetic sequence
  MISC_FEATURE (1)..(5): linker
GGLSL

SEQ ID NO: 106 - protein, 5 aa, Artificial Sequence
  Synthetic sequence
  MISC_FEATURE (1)..(5): linker
GGLLG

SEQ ID NO: 107 - protein, 5 aa, Artificial Sequence
  Synthetic sequence
  MISC_FEATURE (1)..(5): linker
GLGSL

SEQ ID NO: 108 - protein, 5 aa, Artificial Sequence
  Synthetic sequence
  MISC_FEATURE (1)..(5): linker
LGLSS

SEQ ID NO: 109 - protein, 5 aa, Artificial Sequence
  Synthetic sequence
  MISC_FEATURE (1)..(5): linker
GGLLS

SEQ ID NO: 110 - protein, 10 aa, Artificial Sequence
  Synthetic sequence
  MISC_FEATURE (1)..(10): linker
LGGGSGGGGS

SEQ ID NO: 111 - protein, 10 aa, Artificial Sequence
  Synthetic sequence
  MISC_FEATURE (1)..(10): linker
GLGGSGGGGS

SEQ ID NO: 112 - protein, 10 aa, Artificial Sequence
  Synthetic sequence
  MISC_FEATURE (1)..(10): linker
GGLGSGGGGS

SEQ ID NO: 113 - protein, 10 aa, Artificial Sequence
  Synthetic sequence
  MISC_FEATURE (1)..(10): linker
GGGLSGGGGS

SEQ ID NO: 114 - protein, 10 aa, Artificial Sequence
  Synthetic sequence
  MISC_FEATURE (1)..(10): linker
GGGGLGGGGS

SEQ ID NO: 115 - protein, 10 aa, Artificial Sequence
  Synthetic sequence
  MISC_FEATURE (1)..(10): linker
LGGSGGGGSG

SEQ ID NO: 116 - protein, 10 aa, Artificial Sequence
  Synthetic sequence
  MISC_FEATURE (1)..(10): linker
GLGSGGGGSG

SEQ ID NO: 117 - protein, 10 aa, Artificial Sequence
  Synthetic sequence
  MISC_FEATURE (1)..(10): linker
GGLSGGGGSG

SEQ ID NO: 118 - protein, 10 aa, Artificial Sequence
  Synthetic sequence
  MISC_FEATURE (1)..(10): linker
GGGLGGGGSG

SEQ ID NO: 119 - protein, 10 aa, Artificial Sequence
  Synthetic sequence
  MISC_FEATURE (1)..(10): linker
GGGSLGGGSG

SEQ ID NO: 120 - protein, 10 aa, Artificial Sequence
  Synthetic sequence
  MISC_FEATURE (1)..(10): linker
LGGSSGGGSS

SEQ ID NO: 121 - protein, 10 aa, Artificial Sequence
  Synthetic sequence
  MISC_FEATURE (1)..(10): linker
GLGSSGGGSS

SEQ ID NO: 122 - protein, 10 aa, Artificial Sequence
  Synthetic sequence
  MISC_FEATURE (1)..(10): linker
GGLSSGGGSS

SEQ ID NO: 123 - protein, 10 aa, Artificial Sequence
  Synthetic sequence
  MISC_FEATURE (1)..(10): linker
GGGLSGGGSS

SEQ ID NO: 124 - protein, 10 aa, Artificial Sequence
  Synthetic sequence
  MISC_FEATURE (1)..(10): linker
GGGSLGGGSS

SEQ ID NO: 125 - protein, 10 aa, Artificial Sequence
  Synthetic sequence
  MISC_FEATURE (1)..(10): linker
LGGGSLGGGS

SEQ ID NO: 126 - protein, 10 aa, Artificial Sequence
  Synthetic sequence
  MISC_FEATURE (1)..(10): linker
GLGGSGLGGS

SEQ ID NO: 127 - protein, 10 aa, Artificial Sequence
  Synthetic sequence
  MISC_FEATURE (1)..(10): linker
GGLGSGGLGS

SEQ ID NO: 128 - protein, 10 aa, Artificial Sequence
  Synthetic sequence
  MISC_FEATURE (1)..(10): linker
GGGLSGGGLS

SEQ ID NO: 129 - protein, 10 aa, Artificial Sequence
  Synthetic sequence
  MISC_FEATURE (1)..(10): linker
GGGGLGGGGL

SEQ ID NO: 130 - protein, 10 aa, Artificial Sequence
  Synthetic sequence
  MISC_FEATURE (1)..(10): linker
LGGSGLGGSG

SEQ ID NO: 131 - protein, 10 aa, Artificial Sequence
  Synthetic sequence
  MISC_FEATURE (1)..(10): linker
GLGSGGLGSG

SEQ ID NO: 132 - protein, 10 aa, Artificial Sequence
  Synthetic sequence
  MISC_FEATURE (1)..(10): linker
GGLSGGGLSG

SEQ ID NO: 133 - protein, 10 aa, Artificial Sequence
  Synthetic sequence
  MISC_FEATURE (1)..(10): linker
GGGLGGGGLG

SEQ ID NO: 134 - protein, 10 aa, Artificial Sequence
  Synthetic sequence
  MISC_FEATURE (1)..(10): linker
GGGSLGGGSL

SEQ ID NO: 135 - protein, 10 aa, Artificial Sequence
  Synthetic sequence
  MISC_FEATURE (1)..(10): linker
LGGSSLGGSS

SEQ ID NO: 136 - protein, 10 aa, Artificial Sequence
  Synthetic sequence
  MISC_FEATURE (1)..(10): linker
GLGSSGLGSS

SEQ ID NO: 137 - protein, 10 aa, Artificial Sequence
  Synthetic sequence
  MISC_FEATURE (1)..(10): linker
GGLSSGGLSS

SEQ ID NO: 138 - protein, 6 aa, Artificial Sequence
  Synthetic sequence
  MISC_FEATURE (1)..(6): linker
GSGGGA

SEQ ID NO: 139 - protein, 12 aa, Artificial Sequence
  Synthetic sequence
  MISC_FEATURE (1)..(12): linker
GSGGGAGSGGGA

SEQ ID NO: 140 - protein, 18 aa, Artificial Sequence
  Synthetic sequence
  MISC_FEATURE (1)..(18): linker
GSGGGAGSGGGAGSGGGA

SEQ ID NO: 141 - protein, 24 aa, Artificial Sequence
  Synthetic sequence
  MISC_FEATURE (1)..(24): linker
GSGGGAGSGGGAGSGGGAGSGGGA

SEQ ID NO: 142 - protein, 10 aa, Artificial Sequence
  Synthetic sequence
  MISC_FEATURE (1)..(10): linker
GENLYFQSGG

SEQ ID NO: 143 - protein, 10 aa, Artificial Sequence
  Synthetic sequence
  MISC_FEATURE (1)..(10): linker
SGGGSSGGGS

SEQ ID NO: 144 - protein, 10 aa, Artificial Sequence
  Synthetic sequence
  MISC_FEATURE (1)..(10): linker
SSGGGSSGGG

SEQ ID NO: 145 - protein, 10 aa, Artificial Sequence
  Synthetic sequence
  MISC_FEATURE (1)..(10): linker
GGSGGGGSGG

SEQ ID NO: 146 - protein, 10 aa, Artificial Sequence
  Synthetic sequence
  MISC_FEATURE (1)..(10): linker
GSGSGSGSGS

SEQ ID NO: 147 - protein, 10 aa, Artificial Sequence
  Synthetic sequence
  MISC_FEATURE (1)..(10): linker
GGGSSGGGSG

SEQ ID NO: 148 - protein, 6 aa, Artificial Sequence
  Synthetic sequence
  MISC_FEATURE (1)..(6): linker
GGGSSS

SEQ ID NO: 149 - protein, 15 aa, Artificial Sequence
  Synthetic sequence
  MISC_FEATURE (1)..(15): linker
GGGSSGGGSSGGGSS

SEQ ID NO: 150 - protein, 8 aa, Artificial Sequence
  Synthetic sequence
  MISC_FEATURE (1)..(8): linker
GLGGLAAA

SEQ ID NO: 151 - protein, 16 aa, Artificial Sequence
  Synthetic sequence
  MISC_FEATURE (1)..(16): linker
TQKSLSLSPGKGLGGL

SEQ ID NO: 152 - protein, 13 aa, Artificial Sequence
  Synthetic sequence
  MISC_FEATURE (1)..(13): linker
SLSLSPGKGLGGL

SEQ ID NO: 153 - protein, 5 aa, Artificial Sequence
  Synthetic sequence
  MISC_FEATURE (1)..(5): linker
GPGPG

SEQ ID NO: 154 - protein, 36 aa, Artificial Sequence
  Synthetic sequence
  MISC_FEATURE (1)..(36): linker
GGSAGGSGSGSSGGSSGASGTGTAGGTGSGSGTGSG

SEQ ID NO: 155 - protein, 36 aa, Artificial Sequence
  Synthetic sequence
  MISC_FEATURE (1)..(36): linker
GGSGGGSEGGGSEGGGSEGGGSEGGGSEGGGSGGGS

SEQ ID NO: 156 - protein, 15 aa, Artificial Sequence
  Synthetic sequence
  MISC_FEATURE (1)..(15): linker
EPKSCDTPPPCPRCP

SEQ ID NO: 157 - DNA, 333 bp, Artificial Sequence
  Synthetic sequence
  misc_feature (1)..(333): hinge region derived from IgM
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 158 - DNA, 180 bp, Artificial Sequence
  Synthetic sequence
  misc_feature (1)..(180): trimerization unit
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 159 - protein, 27 aa, Artificial Sequence
  Synthetic sequence
  MISC_FEATURE (1)..(27): trimerization unit
GYIPEAPRDGQAYVRKDGEWVLLSTFL

SEQ ID NO: 160 - DNA, 117 bp, Artificial Sequence
  Synthetic sequence
  misc_feature (1)..(117): tetramerization unit
aagcctctggacggagagtatttcactctccagatccggggccccgaaaggttcgaaatgttccgggagcttaacgaggccttggagctgaaagacgcacaggccggaaaggaaccg

SEQ ID NO: 161 - protein, 5 aa, Artificial Sequence
  Synthetic sequence
  MISC_FEATURE (1)..(5): linker
  MISC_FEATURE (1)..(5): The sequence can be repeated m times, wherein m is an integer from 1 to 5, e.g., 1, 2, 3, 4, or 5.
GGSGG

SEQ ID NO: 162 - protein, 5 aa, Artificial Sequence
  Synthetic sequence
  MISC_FEATURE (1)..(5): linker
  MISC_FEATURE (1)..(5): The sequence can be repeated m times, wherein m is an integer from 1 to 5, e.g., 1, 2, 3, 4, or 5.
GLGGL

SEQ ID NO: 163 - protein, 12 aa, Artificial Sequence
  Synthetic sequence
  MISC_FEATURE (1)..(12): linker
KPEPKPAPAPKP

SEQ ID NO: 164 - protein, 12 aa, Artificial Sequence
  Synthetic sequence
  MISC_FEATURE (1)..(12): linker
AEAAAKEAAAKA

SEQ ID NO: 165 - protein, 7 aa, Artificial Sequence
  Synthetic sequence
  MISC_FEATURE (1)..(7): linker
  MISC_FEATURE (1)..(5): The sequence can be repeated m times, wherein m is an integer from 1 to 5, e.g., 1, 2, 3, 4, or 5.
EAAAKGS

SEQ ID NO: 166 - protein, 15 aa, Artificial Sequence
  Synthetic sequence
  MISC_FEATURE (1)..(15): linker
PSRLEEELRRRLTEP

SEQ ID NO: 167 - protein, 8 aa, Artificial Sequence
  Synthetic sequence
  MISC_FEATURE (1)..(8)
  MISC_FEATURE (1)..(8): linker
SACYCELS

SEQ ID NO: 168 - protein, 8 aa, Artificial Sequence
  Synthetic sequence
  MISC_FEATURE (1)..(8): linker
SIINFEKL

SEQ ID NO: 169 - protein, 13 aa, Artificial Sequence
  Synthetic sequence
  MISC_FEATURE (1)..(13): linker
HCLGKWLGHPDKF

SEQ ID NO: 170 - protein, 29 aa, Artificial Sequence
  Synthetic sequence
  MISC_FEATURE (1)..(29): linker
AHSLERVCHCLGKWLGHPDKFVGITYALT

SEQ ID NO: 171 - protein, 14 aa, Artificial Sequence
  Synthetic sequence
  MISC_FEATURE (1)..(14): linker
NTWTTCQSIAFPSK